(19) United States
(12) Patent Application Publication
(10) Pub. No.: US 2010/0035860 A1
(43) Pub. Date: Feb. 11, 2010

(54) INHIBITORS OF PROTEIN TYROSINE PHOSPHATASE FOR THE PROMOTION OF PHYSIOLOGICAL CARDIAC HYPERTROPHY

(76) Inventors: Jey Raju Jeyaseelan, Acton, MA (US); Mie Abe, Watertown, MA (US)

Correspondence Address: NOVARTIS INSTITUTES FOR BIOMEDICAL RESEARCH, INC., 220 MASSACHUSETTS AVENUE, CAMBRIDGE, MA, 02139, US

(21) Appl. No.: 12/515,519

(22) PCT Filed: Nov. 30, 2007
(86) PCT No.: PCT/US07/86070

§ 371 Date: May 19, 2009

Related U.S. Application Data
(60) Provisional application No. 60/872,326, filed on Dec. 1, 2006.

(51) Int. Cl. A61K 31/433; A61K 31/4725; A61K 31/55; A61P 9/00

(52) U.S. Cl. 514/217; 514/362; 514/314

(57) ABSTRACT

The invention relates to the use of protein tyrosine phosphatase inhibitors for the promotion of physiological cardiac hypertrophy and the treatment of pathological cardiac hypertrophy.

### FIELD OF THE INVENTION

The invention relates to compounds that inhibit protein tyrosine phosphatase (PTP), particularly PTP-1B, and their use in the promotion of physiological cardiac hypertrophy.

### BACKGROUND OF THE INVENTION

Heart failure is a disorder in which the heart pumps blood inadequately, leading to reduced blood flow, back-up (congestion) of blood in the veins and lungs, and other changes that may further weaken the heart.

Heart failure can occur in people of any age, even in young children (especially those born with a heart defect). However, it is much more common among older people, because older people are more likely to have disorders that damage the heart muscle and because age-related changes in the heart tend to make the heart pump less efficiently. Heart failure develops in about 1 of 100 people. The disorder is likely to become more common because people are living longer and because, in some countries, certain risk factors for heart disease (such as smoking, high blood pressure, and a high-fat diet) are affecting more people.

Heart failure does not mean that the heart has stopped; it means that the heart cannot keep up with the work required of it (its workload). However, this definition is remarkably simplified. Heart failure is extremely complex, and no simple definition can encompass its many causes, aspects, forms, and consequences.

Any disorder that directly affects the heart can lead to heart failure, as can some disorders that indirectly affect the heart. Some disorders cause heart failure quickly; others do so only after many years. Some disorders cause systolic dysfunction, impairing the heart's ability to pump out blood, and others cause diastolic dysfunction, impairing the heart's ability to fill with blood. Some disorders, such as high blood pressure and heart valve disorders, can cause both types of dysfunction.

Symptoms of heart failure may begin suddenly, especially if the cause is a heart attack. However, in most people, symptoms develop over days to months. The disorder may stabilize for periods of time but often progresses slowly and insidiously.

People with heart failure feel tired and weak when performing physical activities, because their muscles are not receiving enough blood. In older people, heart failure sometimes causes vague symptoms such as sleepiness, confusion, and disorientation, as well as weakness and fatigue.

Heart failure is characterized by a distinct phenomenon called pathological cardiac hypetrophy, a condition associated with a greater enlargement of cardiac myocyte width than any increase in myocyte length (concentric cellular hypertrophy), leading to ventricular wall and septum thickening with net increase in ventricular chamber dimensions.

However, not all forms of cardiac hypertrophy are detrimental, as extensive aerobic conditioning through exercise or pregnancy induces a state of physiological cardiac growth that is thought to be beneficial in the long term. This state is called physiological cardiac hypetrophy and is associated with a greater lengthening of cardiac myocytes than any increase in myocyte width (eccentric cellular hypertrophy), leading to a uniform profile of ventricular wall and septum growth that is matched with an increase in chamber dimension.

Other than behavioral modification (e.g., diet and exercise), there is no pharmaceutical intervention that can directly affect cardiac myocytes to promote physiological cardiac hypertrophy and consequently treat pathological cardiac hypertrophy.

For a review of cardiac hypetrophy, see, e.g., Heineke and Molkentin, Nat Rev Mol Cell Biol 7:589-598, 2006.

### SUMMARY OF THE INVENTION

The invention is based on the discovery that PTP inhibitors directly modify cardiac myocyte physiology to promote physiological cardiac hypertrophy in a mammal such as a human individual or patient. Through promotion of physiological cardiac hypertrophy, the methods of the invention include treatment or prevention of pathological cardiac hypertrophy or any condition that is characterized by pathological cardiac hypertrophy, such as heart failure, myocardial infarction, or a cardiomyopathy, for example, dilated, familial, or ischemic cardiomyopathy.

Accordingly, the invention includes a method of promoting physiological cardiac hypertrophy in an individual by identifying an individual having or suspected of having pathological cardiac hypertrophy and contacting the cardiomyocytes of the individual with a therapeutically effective amount of a PTP inhibitor sufficient to promote physiological hypertrophy. The invention also includes the use of a PTP inhibitor in the manufacture of a medicament for promotion of physiological cardiac hypertrophy in an individual. Such individuals may be diagnosed with heart failure, diastolic or systolic dysfunction, myocardial infarction, or dilated, familial, or ischemic cardiomyopathy.

The PTP inhibitors can be administered systemically (e.g., by oral or intravenous routes) or directly to the heart (e.g., by direct injection or by use of a catheter in a coronary artery that supplies the heart muscle).

Any suitable PTP inhibitor, such as described in the next section, can be used to promote physiological cardiac hypertrophy. Since it is known that insulin-like growth factor-1 (IGF1) has beneficial effects on heart muscle, IGF1 can be co-administered along with the PTP inhibitor in the methods of the invention, especially under circumstances in which the individual has low IGF1 levels.

All cited references or documents are hereby incorporated by reference.

### DETAILED DESCRIPTION OF THE INVENTION

The invention relates to the treatment of diseases characterized by pathological cardiac hypertrophy, in particular heart failure, by promoting physiological cardiac hypertrophy. Any PTP inhibitor can be used in the methods of the invention, for example, the inhibitors described in US patents and patent application publications U.S. Pat. Nos. 7,115,624; 7,078,425; 7,022,730; 6,911,468; and 2005/0090502. In addition, specific inhibitors of PTP, particularly PTP-1B and T-cell PTP, can include the categories of compounds and specific compounds therein, as described below.

### DEFINITIONS FOR CHEMICAL STRUCTURES

Listed below are definitions of various terms used to describe the compounds of the instant invention. These definitions apply to the terms as they are used throughout the specification unless they are otherwise limited in specific instances either individually or as part of a larger group. In general, whenever an alkyl group is referred to as a part of the structure, an optionally substituted alkyl is also intended.

Accordingly, the term “optionally substituted alkyl” refers to unsubstituted or substituted straight or branched chain hydrocarbon groups having 1 to 20 carbon atoms, preferably 1 to 8 carbon atoms. Exemplary unsubstituted alkyl groups include methyl, ethyl, propyl, isopropyl, n-butyl, t-butyl, isobutyl, pentyl, hexyl, isohexyl, heptyl, 4,4-dimethylpentyl, octyl and the like. Substituted alkyl groups include, but are not limited to, alkyl groups substituted by one or more of the following groups: halogen, hydroxy, cycloalkyl, cycloalkoxy, acyl, acyloxy, alkoxy, alkyloxyalkoxy, alkanoyloxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaraloxy, heterocyclyl and heterocyclyloxy including indolyl, imidazolyl, furyl, thienyl, thiazolyl, pyrrolidyl, pyridyl, pyrimidyl, piperidyl, morpholinyl and the like.

The term “lower alkyl” refers to any of the above alkyl groups as described above having 1 to 7, preferably 1 to 4 carbon atoms.

The term “halogen” or “halo” refers to fluorine, chlorine, bromine and iodine.

The term “alkenyl” refers to any of the above alkyl groups having at least 2 carbon atoms and containing a carbon to carbon double bond at the point of attachment. Groups having 2 to 8 carbon atoms are preferred.

The term “alkynyl” refers to any of the above alkyl groups having at least two carbon atoms and containing a carbon to carbon triple bond at the point of attachment. Groups having 2 to 8 carbon atoms are preferred.

The term “alkylene” refers to a straight-chain bridge of 1-6 carbon atoms connected by single bonds, e.g., —(CH2)x-, wherein x is 1-6, which may be interrupted with one or more heteroatoms selected from O, S, S(O), S(O)2 or NR″, wherein R″ may be hydrogen, alkyl, cycloalkyl, aryl, heterocyclyl, aralkyl, heteroaralkyl, acyl, carbamoyl, sulfonyl, alkoxycarbonyl, aryloxycarbonyl or aralkoxycarbonyl and the like; and the alkylene may further be substituted with one or more substituents selected from hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C1-8)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl, heterocyclyloxy and the like.

The term “cycloalkyl” refers to optionally substituted monocyclic, bicyclic or tricyclic hydrocarbon groups of 3 to 12 carbon atoms, each of which may be substituted by one or more substituents such as alkyl, halo, oxo, hydroxy, alkoxy, alkanoyl, acylamino, carbamoyl, alkylamino, dialkylamino, thiol, alkylthio, nitro, cyano, carboxy, carboxyalkyl, alkoxycarbonyl, sulfonyl, sulfonamido, sulfamoyl, heterocyclyl and the like.

Exemplary monocyclic hydrocarbon groups include but are not limited to cyclopropyl, cyclobutyl, cyclopentyl, cyclopentenyl, cyclohexyl and cyclohexenyl and the like. Exemplary bicyclic hydrocarbon groups include bornyl, indyl, hexahydroindyl, tetrahydronaphthyl, decahydronaphthyl, bicyclo[2.1.1]hexyl, bicyclo[2.2.1]heptyl, bicyclo[2.2.1]heptenyl, 6,6-dimethylbicyclo[3.1.1]heptyl, 2,6,6-trimethylbicyclo[3.1.1]heptyl, bicyclo[2.2.2]octyl and the like. Exemplary tricyclic hydrocarbon groups include adamantyl and the like.

The term “alkoxy” refers to alkyl-O—.

The term “alkanoyl” refers to alkyl-C(O)—.

The term “alkanoyloxy” refers to alkyl-C(O)—O—.

The terms “alkylamino” and “dialkylamino” refer to alkyl-NH— and (alkyl)₂N—, respectively.

The term “alkanoylamino” refers to alkyl-C(O)—NH—.

The term “alkylthio” refers to alkyl-S—.

The term “alkylaminothiocarbonyl” refers to alkyl-NHC(S)—.

The term “trialkylsilyl” refers to (alkyl)₃Si—.

The term “trialkylsilyloxy” refers to (alkyl)₃SiO—.

The term “alkylthiono” refers to alkyl-S(O)—.

The term “alkylsulfonyl” refers to alkyl-S(O)₂—.

The term “alkoxycarbonyl” refers to alkyl-O—C(O)—.

The term “alkoxycarbonyloxy” refers to alkyl-O—C(O)O—.

The term “carboxycarbonyl” refers to HO—C(O)C(O)—.

The term “carbamoyl” refers to H₂NC(O)—, alkyl-NHC(O)—, (alkyl)₂NC(O)—, aryl-NHC(O)—, alkyl(aryl)-NC(O)—, heteroaryl-NHC(O)—, alkyl(heteroaryl)-NC(O)—, aralkyl-NHC(O)—, alkyl(aralkyl)-NC(O)— and the like.

The term “sulfamoyl” refers to H2NS(O)₂—, alkyl-NHS(O)₂—, (alkyl)₂NS(O)₂—, aryl-NHS(O)₂—, alkyl(aryl)-NS(O)₂—, (aryl)₂NS(O)₂—, heteroaryl-NHS(O)₂—, aralkyl-NHS(O)₂—, heteroaralkyl-NHS(O)₂— and the like.

The term “sulfonamido” refers to alkyl-S(O)₂—NH—, aryl-S(O)₂—NH—, aralkyl-S(O)₂—NH—, heteroaryl-S(O)₂—NH—, heteroaralkyl-S(O)₂—NH—, alkyl-S(O)₂—N(alkyl)-, aryl-S(O)₂—N(alkyl)-, aralkyl-S(O)₂—N(alkyl)-, heteroaryl-S(O)₂—N(alkyl)-, heteroaralkyl-S(O)₂—N(alkyl)- and the like.

The term “sulfonyl” refers to alkylsulfonyl, arylsulfonyl, heteroarylsulfonyl, aralkylsulfonyl, heteroaralkylsulfonyl and the like.

The term “sulfonate” or “sulfonyloxy” refers to alkyl-S(O)₂—O—, aryl-S(O)₂—O—, aralkyl-S(O)₂—O—, heteroaryl-S(O)₂—O—, heteroaralkyl-S(O)₂—O— and the like.

The term “optionally substituted amino” refers to a primary or secondary amino group which may optionally be substituted by a substituent such as acyl, sulfonyl, alkoxycarbonyl, cycloalkoxycarbonyl, aryloxycarbonyl, heteroaryloxycarbonyl, aralkoxycarbonyl, heteroaralkoxycarbonyl, carboxycarbonyl, carbamoyl, alkylaminothiocarbonyl, arylaminothiocarbonyl and the like.

The term “aryl” refers to monocyclic or bicyclic aromatic hydrocarbon groups having 6 to 12 carbon atoms in the ring portion, such as phenyl, naphthyl, tetrahydronaphthyl, biphenyl and diphenyl groups, each of which may optionally be substituted by one to five substituents such as alkyl, trifluoromethyl, halo, hydroxy, alkoxy, acyl, alkanoyloxy, optionally substituted amino, thiol, alkylthio, nitro, cyano, carboxy, carboxyalkyl, alkoxycarbonyl, carbamoyl, alkylthiono, sulfonyl, sulfonamido, sulfonate, heterocyclyl and the like.

The term “monocyclic aryl” refers to optionally substituted phenyl as described under aryl.

The term “aralkyl” refers to an aryl group bonded directly through an alkyl group, such as benzyl.

The term “aralkanoyl” refers to aralkyl-C(O)—.

The term “aralkylthio” refers to aralkyl-S—.

The term “aralkoxy” refers to an aryl group bonded directly through an alkoxy group.

The term “arylsulfonyl” refers to aryl S(O)₂.

The term “arylthio” refers to aryl-S—.

The term “aroyl” refers to aryl-C(O)—.

The term “aroylamino” refers to aryl-C(O)—NH—.

The term “aryloxycarbonyl” refers to aryl-O—C(O)—.

The term “heterocyclyl” or “heterocyclo” refers to an optionally substituted, aromatic, or a partially or fully saturated nonaromatic cyclic group, for example, which is a 4- to 7-membered monocyclic, 7- to 12-membered bicyclic, or 10- to 15-membered tricyclic ring system, which has at least one heteroatom in at least one carbon atom containing ring. Each ring of the heterocyclic group containing a heteroatom may have 1, 2 or 3 heteroatoms selected from nitrogen atoms, oxygen atoms and sulfur atoms, where the nitrogen and sulfur heteroatoms may also optionally be oxidized. The heterocyclic group may be attached at a heteroatom or a carbon atom.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2 oxopiperazinyl, 2 oxopiperidinyl, 2 oxopyrrolodinyl, 2 oxoazepinyl, azepinyl, 4 piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl sulfone, 1,3 dioxolane and tetrahydro 1,1 dioxothienyl, 1,1,4-trioxo-1,2,5-thiadiazolidin-2-yl and the like.

Exemplary bicyclic heterocyclic groups include indolyl, dihydroidolyl, benzothiazolyl, benzoxazinyl, benzoxazolyl, benzothienyl, benzothiazinyl, quinuclidinyl, quinolinyl, tetrahydroquinolinyl, decahydroquinolinyl, isoquinolinyl, tetrahydroisoquinolinyl, decahydroisoquinolinyl, benzimidazolyl, benzopyranyl, indolizinyl, benzofuryl, chromonyl, coumarinyl, benzopyranyl, benzodiazepinyl, cinnolinyl, quinoxalinyl, indazolyl, pyrrolopyridyl, furopyridinyl (such as furo[2,3 c]pyridinyl, furo[3,2 b]-pyridinyl] or furo[2,3 b]pyridinyl), dihydroisoindolyl, 1,3-dioxo-1,3-dihydroisoindol-2-yl, dihydroquinazolinyl (such as 3,4 dihydro-4 oxo-quinazolinyl), phthalazinyl and the like.

Exemplary tricyclic heterocyclic groups include carbazolyl, dibenzoazepinyl, dithienoazepinyl, benzindolyl, phenanthrolinyl, acridinyl, phenanthridinyl, phenoxazinyl, phenothiazinyl, xanthenyl, carbolinyl and the like.

The term “heterocyclyl” includes substituted heterocyclic groups. Substituted heterocyclic groups refer to heterocyclic groups that are substituted with 1, 2 or 3 substituents selected from the group consisting of the following:

(a) optionally substituted alkyl;
(b) hydroxy (or protected hydroxy);
(c) halo;
(d) oxo (i.e. ═O);
(e) optionally substituted amino, alkylamino or dialkylamino;
(f) alkoxy;
(g) cycloalkyl;
(h) carboxy;
(i) heterocyclooxy;
(j) alkoxycarbonyl, such as unsubstituted lower alkoxycarbonyl;
(k) mercapto;
(l) nitro;
(m) cyano;
(n) sulfamoyl or sulfonamido;
(o) alkylcarbonyloxy;
(p) arylcarbonyloxy;
(q) arylthio;
(r) aryloxy;
(s) alkylthio;
(t) formyl;
(u) carbamoyl;
(v) aralkyl; and
(w) aryl substituted with alkyl, cycloalkyl, alkoxy, hydroxy, amino, acylamino, alkylamino, dialkylamino or halo.

The term “heterocyclooxy” denotes a heterocyclic group bonded through an oxygen bridge. The term “heteroaryl” refers to an aromatic heterocycle, for example monocyclic or bicyclic aryl, such as pyrrolyl, pyrazolyl, imidazolyl, oxazolyl, isoxazolyl, thiazolyl, isothiazolyl, furyl, thienyl, pyridyl, pyrazinyl, pyrimidinyl, pyridazinyl, indolyl, benzothiazolyl, benzoxazolyl, benzothienyl, quinolinyl, isoquinolinyl, benzimidazolyl, benzofuryl, and the like, optionally substituted by e.g. lower alkyl, lower alkoxy or halo.

The term “heteroarylsulfonyl” refers to heteroaryl S(O)₂

The term “heteroaroyl” refers to heteroaryl-C(O)—.

The term “heteroaroylamino” refers to heteroaryl-C(O)NH—.

The term “heteroaralkyl” refers to a heteroaryl group bonded through an alkyl group.

The term “heteroaralkanoyl” refers to heteroaralkyl-C(O)—.

The term “heteroaralkanoylamino” refers to heteroaralkyl-C(O)NH—.

The term “acyl” refers to alkanoyl, cycloalkanoyl, aroyl, heteroaroyl, aralkanoyl, heteroaralkanoyl and the like.

The term “acyloxy” refers to alkanoyloxy, cycloalkanoyloxy, aroyloxy, heteroaroyloxy, aralkanoyloxy, heteroaralkanoyloxy and the like.

The term “acylamino” refers to alkanoylamino, cycloalkanoylamino, aroylamino, heteroaroylamino, aralkanoylamino, heteroaralkanoylamino and the like.

The term “esterified carboxy” refers to optionally substituted alkoxycarbonyl, cycloalkoxycarbonyl, aryloxycarbonyl, aralkoxycarbonyl, heterocyclooxycarbonyl and the like.

Pharmaceutically acceptable salts of any compound useful in the present invention refer to salts formed with bases, namely cationic salts such as alkali and alkaline earth metal salts, such as sodium, lithium, potassium, calcium, magnesium, as well as ammonium salts, such as ammonium, trimethylammonium, diethylammonium, and tris(hydroxymethyl)-methyl-ammonium salts, and salts with amino acids.

Similarly acid addition salts, such as those formed with mineral acids, organic carboxylic acids and organic sulfonic acids e.g. hydrochloric acid, maleic acid and methanesulfonic acid, are possible provided a basic group, such as pyridyl, constitutes part of the structure.

In starting compounds and intermediates which are converted to the compounds useful in the invention in a manner described herein, functional groups present, such as amino, thiol, carboxyl, and hydroxy groups, are optionally protected by conventional protecting groups that are common in preparative organic chemistry. Protected amino, thiol, carboxyl, and hydroxyl groups are those that can be converted under mild conditions into free amino thiol, carboxyl and hydroxyl groups without the molecular framework being destroyed or other undesired side reactions taking place.

The purpose of introducing protecting groups is to protect the functional groups from undesired reactions with reaction components under the conditions used for carrying out a desired chemical transformation. The need and choice of protecting groups for a particular reaction is known to those skilled in the art and depends on the nature of the functional group to be protected (hydroxyl group, amino group, etc.), the structure and stability of the molecule of which the substituent is a part and the reaction conditions.

Well known protecting groups that meet these conditions and their introduction and removal are described, for example, in McOmie, “Protective Groups in Organic Chemistry”, Plenum Press, London, N.Y. (1973); and Greene and Wuts, “Protective Groups in Organic Synthesis”, John Wiley and Sons, Inc, New York (1999).

The above mentioned reactions are carried out according to standard methods, in the presence or absence of diluent, preferably such as are inert to the reagents and are solvents thereof, of catalysts, condensing or said other agents respectively and/or inert atmospheres, at low temperatures, room temperature or elevated temperatures (preferably at or near the boiling point of the solvents used), and at atmospheric or super-atmospheric pressure.

The invention further includes any variant of the present processes, in which an intermediate product obtainable at any stage thereof is used as starting material and the remaining steps are carried out, or in which the starting materials are formed in situ under the reaction conditions, or in which the reaction components are used in the form of their salts or optically pure antipodes.

Compounds useful in the invention and intermediates can also be converted into each other according to methods generally known per se.

Depending on the choice of starting materials and methods, the compounds may be in the form of one of the possible isomers or mixtures thereof, for example, as substantially pure geometric (cis or trans) isomers, optical isomers (enantiomers, antipodes), racemates, or mixtures thereof. The aforesaid possible isomers or mixtures thereof are within the purview of this invention.

Any resulting mixtures of isomers can be separated on the basis of the physico-chemical differences of the constituents, into the pure geometric or optical isomers, diastereoisomers, racemates, for example by chromatography and/or fractional crystallization.

Any resulting racemates of final products or intermediates can be resolved into the optical antipodes by known methods, e.g. by separation of the diastereoisomeric salts thereof, obtained with an optically active acid or base, and liberating the optically active acidic or basic compound. The carboxylic acid intermediates can thus be resolved into their optical antipodes e.g. by fractional crystallization of D- or L-(alpha-methylbenzylamine, cinchonidine, cinchonine, quinine, quinidine, ephedrine, dehydroabietylamine, brucine or strychnine)-salts. Racemic products can also be resolved by chiral chromatography, e.g. high pressure liquid chromatography using a chiral adsorbent.

Finally, compounds useful in the invention are either obtained in the free form, as a salt thereof if salt forming groups are present or as prodrug derivatives thereof.

In particular, the NH-group of the 1,1-dioxo-1,2,5-thiadiazolidin-3-one moiety, may be converted into salts with pharmaceutically acceptable bases. Salts may be formed using conventional methods, advantageously in the presence of an ethereal or alcoholic solvent, such as a lower alkanol. From the solutions of the latter, the salts may be precipitated with ethers, e.g. diethyl ether. Resulting salts may be converted into the free compounds by treatment with acids. These or other salts can also be used for purification of the compounds obtained.

Compounds useful in the invention having basic groups can be converted into acid addition salts, especially pharmaceutically acceptable salts. These are formed, for example, with inorganic acids, such as mineral acids, for example sulfuric acid, a phosphoric or hydrohalic acid, or with organic carboxylic acids, such as (C₁₋₄)alkanecarboxylic acids which, for example, are unsubstituted or substituted by halogen, for example acetic acid, such as saturated or unsaturated dicarboxylic acids, for example oxalic, succinic, maleic or fumaric acid, such as hydroxy-carboxylic acids, for example glycolic, lactic, malic, tartaric or citric acid, such as amino acids, for example aspartic or glutamic acid, or with organic sulfonic acids, such as (C₁₋₄)alkyl-sulfonic acids (for example methanesulfonic acid) or arylsulfonic acids which are unsubstituted or substituted (for example by halogen). Preferred are salts formed with hydrochloric acid, methanesulfonic acid and maleic acid.

Prodrug derivatives of any compound of the present invention are derivatives of said compounds which following administration release the parent compound in vivo via some chemical or physiological process, e.g., a prodrug on being brought to the physiological pH or through enzyme action is converted to the parent compound. Exemplary prodrug derivatives are, e.g., esters of free carboxylic acids and S-acyl and O-acyl derivatives of thiols, alcohols or phenols, wherein acyl has a meaning as defined herein. Preferred are pharmaceutically acceptable ester derivatives convertible by solvolysis under physiological conditions to the parent carboxylic acid, e.g., lower alkyl esters, cycloalkyl esters, lower alkenyl esters, benzyl esters, mono- or di-substituted lower alkyl esters, such as the ω-(amino, mono- or di-lower alkylamino, carboxy, lower alkoxycarbonyl)-lower alkyl esters, the α-(lower alkanoyloxy, lower alkoxycarbonyl or di-lower alkylaminocarbonyl)-lower alkyl esters, such as the pivaloyloxymethyl ester and the like conventionally used in the art.

In view of the close relationship between the free compounds, the prodrug derivatives and the compounds in the form of their salts, whenever a compound is referred to in this context, a prodrug derivative and a corresponding salt is also intended, provided such is possible or appropriate under the circumstances.

The compounds, including their salts, can also be obtained in the form of their hydrates, or include other solvents used for their crystallization.

Thus, the pharmacologically active compounds useful in the invention may be employed in the manufacture of pharmaceutical compositions comprising an effective amount thereof in conjunction or admixture with excipients or carriers suitable for either enteral or parenteral application. Preferred are tablets and gelatin capsules comprising the active ingredient together with:

a) diluents, e.g., lactose, dextrose, sucrose, mannitol, sorbitol, cellulose and/or glycine;
b) lubricants, e.g., silica, talcum, stearic acid, its magnesium or calcium salt and/or polyethyleneglycol; for tablets also
c) binders, e.g., magnesium aluminum silicate, starch paste, gelatin, tragacanth, methylcellulose, sodium carboxymethylcellulose and or polyvinylpyrrolidone; if desired
d) disintegrants, e.g., starches, agar, alginic acid or its sodium salt, or effervescent mixtures; and/or
e) absorbants, colorants, flavors and sweeteners. Injectable compositions are preferably aqueous isotonic solutions or suspensions, and suppositories are advantageously prepared from fatty emulsions or suspensions.

Said compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Said compositions are prepared according to conventional mixing, granulating or coating methods, respectively, and contain about 0.1-75%, preferably about 1-50%, of the active ingredient.

Suitable formulations for transdermal application include a therapeutically effective amount of a compound of the invention with carrier. Advantageous carriers include absorbable pharmacologically acceptable solvents to assist passage through the skin of the host. Characteristically, transdermal devices are in the form of a bandage comprising a backing member, a reservoir containing the compound optionally with carriers, optionally a rate controlling barrier to deliver the compound of the skin of the host at a controlled and predetermined rate over a prolonged period of time, and means to secure the device to the skin.

The pharmaceutical compositions useful in the invention may contain a therapeutically effective amount of a compound as defined above, either alone or in a combination with another therapeutic agent, e.g., each at an effective therapeutic dose as reported in the art. One such compound that can be administered along with the PTP inhibitor is human insulin-like growth factor 1 or IGF1, however formulated or stabilized, such as IPLEX™ as developed by Insmed Inc or as described in US 2006/0166328.

The structure of the therapeutic agents identified by code numbers, generic or trade names may be taken from the actual edition of the standard compendium “The Merck Index” or from databases, e.g., Patents International (e.g. IMS World Publications). The corresponding content thereof is hereby incorporated by reference.

As used throughout the specification and in the claims, the term “treatment” embraces all the different forms or modes of treatment as known to those of the pertinent art and in particular includes preventive, curative, delay of progression and palliative treatment.

The above-cited properties are demonstrable in vitro and in vivo tests, using advantageously mammals, e.g., mice, rats, dogs, monkeys or isolated organs, tissues and preparations thereof. Said compounds can be applied in vitro in the form of solutions, e.g. preferably aqueous solutions, and in vivo either enterally, parenterally, advantageously intravenously, e.g. as a suspension or in aqueous solution. The dosage in vitro may range between about 10⁻³ molar and 10⁻¹⁰ molar concentrations. A therapeutically effective amount in vivo may range depending on the route of administration, between about 1 and 500 mg/kg, preferably between about 5 and 100 mg/kg.

The activity of a compound according to the invention may be assessed by the following methods or by following methods well described in the art (e.g. Peters G. et al. J. Biol. Chem, 2000, 275, 18201-09).

For example, the PTP-1B inhibitory activity in vitro may be determined as follows:

Assessment of human PTP-1B (hPTP-1B) activity in the presence of various agents is determined by measuring the amount of inorganic phosphate released from a phosphopeptide substrate using a 96-well microtiter plate format. The assay (100 μL) is performed in an assay buffer comprised of 50 mM TRIS (pH 7.5), 50 mM NaCl, 3 mM DTT at ambient temperature. The assay is typically performed in the presence of 0.4% dimethyl sulfoxide (DMSO). However, concentrations as high as 10% are used with certain poorly soluble compounds. A typical reaction is initiated by the addition of 0.4 pmoles of hPTP-1B (amino acids 1-411) to wells containing assay buffer, 3 nmoles of the synthetic phosphopeptide substrate (GNGDpYMPMSPKS), and the test compound. After 10 min, 180 μL malachite green reagent (0.88 mM malachite green, 8.2 mM ammonium molybdate, aqueous 1 N HCl, and 0.01% Triton X-100) is added to terminate the reaction. Inorganic phosphate, a product of the enzyme reaction, is quantitiated after 15 min as the green color resulting from complexing with the Malichite reagent and is determined as an A₆₂₀ using a Molecular Devices (Sunnyvale, Calif.) SpectraMAX Plus spectrophotometer. Test compounds are solubilized in 100% DMSO (Sigma, D-8779) and diluted in DMSO. Activity is defined as the net change in absorbance resulting from the activity of the uninhibited hPTP-1B_{[1-411}] minus that of a tube with acid-inactivated hPTP-1B_{[1-411]}.

The hPTP-1B_{[1-411]} is cloned by PCR from a human hippocampal cDNA library (Clonetech) and inserted into a pET 19-b vector (Novagen) at the Nco1 restriction site. E. coli strain BL21 (DE3) is transformed with this clone and stored as a stock culture in 20% glycerol at −80° C. For enzyme production, a stock culture is inoculated into LB/Amp and grown at 37° C. Expression of PTP-1B is initiated by induction with 1 mM IPTG after the culture had reached an OD₆₀₀=0.6. After 4 h, the bacterial pellet is collected by centrifugation. Cells are resuspended in 70 mL lysis buffer (50 mM Tris, 100 mM NaCl, 5 mM DTT, 0.1% Triton X-100, pH7.6), incubated on ice for 30 min then sonicated (4×10 sec bursts at full power). The lysate is centrifuged at 100,000×g for 60 min and the supernatant is buffer exchanged and purified on a cation exchange POROS 20SP column followed by an anion exchange Source 30Q (Pharmacia) column, using linear NaCl gradient elutions. Enzyme is pooled, adjusted to 1 mg/mL and frozen at −80° C.

Alternatively, the assessment of human PTP-1B activity in the presence of various agents may be determined by measuring the hydrolysis products of known competing substrates. For example, cleavage of substrate para-nitrophenylphosphate (pNPP) results in the release of the yellow-colored para-nitrophenol (pNP) which can be monitored in real time using a spectrophotometer. Likewise, the hydrolysis of the fluorogenic substrate 6,8-difluoro-4-methylumbelliferyl phosphate ammonium salt (DiFMUP) results in the release of the fluorescent DiFMU which can be readily followed in a continuous mode with a fluorescence reader (Anal. Biochem. 273, 41, 1999; Anal. Biochem. 338, 32, 2005):

pNPP Assay

Compounds are incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 for 5 min at room temperature. The reaction is initiated by the addition of pNPP (2 mM final concentration) and run for 120 min at room temperature. Reactions are quenched with 5 N NaOH. Absorbance at 405 nm is measured using any standard 384 well plate reader.

DiFMUP Assay Compounds are incubated with 1 nM recombinant human PTP-1B_{[1-298]} or PTP-1B_{[1-322]} in buffer (50 mM Hepes, pH 7.0, 50 mM KCl, 1 mM EDTA, 3 mM DTT, 0.05% NP-40 (or 0.001% BSA) for 5 min at room temperature. The reaction is initiated by the addition of DiFMUP (6 μM final concentration) and run kinetically on fluorescence plate reader at 355 nm excitation and 460 nm emission wavelengths. Reaction rates over 15 min are used to calculate inhibition.

PTP-1B_{[1-298]} is expressed in E. coli BL21(DE3) containing plasmids constructed using pET19b vectors (Novagen). The bacteria is grown in minimal media using an “On Demand” Fed-batch strategy. Typically, a 5.5 liter fermentation is initiated in Fed-batch mode and grown overnight unattended at 37° C. Optical densities varied between 20-24 OD₆₀₀ and the cultures are induced at 30° C. with IPTG to a final concentration of 0.5 mM. The bacterial cells are harvested 8 hours later and yield 200-350 gm (wet weight). The cells are frozen as pellets and stored at −80° C. until use. All steps are performed at 4° C. unless noted. Cells (˜15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase I. The cells are lysed by sonication (4×10 second burst, full power) using a Virsonic 60 (Virtus). The pellet is collected at 35,000×g, resuspended in 25 mL of lysis buffer using a Polytron and collected as before. The two supernatants are combined and centrifuged for 30 min at 100,000×g. The soluble lysate could be stored at this stage at −80° C. or used for further purification. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. An analytical column (4.6×100 mm) is run in a similar fashion except the flow rate was reduced to 10 mL/min. Protein is eluted from the column using a linear salt gradient (75-500 mM NaCl in 25 CV). Fractions containing PTP-1B_{[1-298]} are identified and pooled according to SDS-PAGE analyses. Final purification is performed using Sephacryl S-100 HR (Pharmacia). The column (2.6×35 cm) is equilibrated with 50 mM HEPES, 100 mM NaCl, 3 mM DTT, pH 7.5 and run at a flow rate of 2 mL/min. The final protein is pooled and concentrated to 5 mg/mL using an Ultrafree-15 concentrator (Millipore) with a MWCO 10,000. The concentrated protein is stored at −80° C. until use.

Competitive binding to the active site of the enzyme can be determined as follows: Ligand binding is detected by acquiring ¹H-¹⁵N HSQC spectra on 250 μL of 0.15 mM PTP-1B_{[1-298]} in the presence and absence of added compound (1-2 mM). The binding is determined by the observation of ¹⁵N- or ¹H-amide chemical shift changes in two dimensional HSQC spectra upon the addition of a compound to ¹⁵N-label protein. Because of the ¹⁵N spectral editing, no signal from the ligand is observed, only protein signals. Thus, binding can be detected at high compound concentrations. Compounds which caused a pattern of chemical shift changes similar to the changes seen with known active site binders are considered positive.

All proteins are expressed in E. coli BL21 (DE3) containing plasmids constructed using pET19b vectors (Novagen). Uniformly ¹⁵N-labeled PTP-1B₁₋₂₉₈ is produced by growth of bacteria on minimal media containing ¹⁵N-labeled ammonium chloride. All purification steps are performed at 4° C. Cells (˜15 g) are thawed briefly at 37° C. and resuspended in 50 mL of lysis buffer containing 50 mM Tris-HCl, 150 mM NaCl, 5 mM DTT, pH 8.0 containing one tablet of Complete (EDTA-free) protease cocktail (Boehringer Mannheim), 100 μM PMSF and 100 μg/mL DNase 1. The cells are lysed by sonication. The pellet is collected at 35,000×g, resuspended in 25 mL of lysis buffer using a Polytron and collected as before. The two supernatants are combined and centrifuged for 30 min at 100,000×g. Diafiltration using a 10 kD MWCO membrane is used to buffer exchange the protein and reduce the NaCl concentration prior to cation exchange chromatography. Diafiltration buffer contained 50 mM MES, 75 mM NaCl, 5 mM DTT, pH 6.5. Soluble supernatant is then loaded onto a POROS 20 SP (1×10 cm) column equilibrated with cation exchange buffer (50 mM MES and 75 mM NaCl, pH 6.5) at a rate of 20 mL/min. Protein is eluted from the column using a linear salt gradient (75-500 mM NaCl in 25 CV). Fractions containing PTP-1B's are identified and pooled according to SDS-PAGE analyses. PTP-1B₁₋₂₉₈ is further purified by anion exchange chromatography using a POROS 20 HQ column (1×10 cm). The pool from cation exchange chromatography is concentrated and buffer exchanged in 50 mM Tris-HCl, pH 7.5 containing 75 mM NaCl and 5 mM DTT. Protein is loaded onto column at 20 mL/min and eluted using a linear NaCl gradient (75-500 mM in 25 CV). Final purification is performed using Sephacryl S-100 HR (Pharmacia)(50 mM HEPES, 100 mM NaCl, 3 mM DTT, pH 7.5). The NMR samples are composed of uniformly ¹⁵N-labeled PTP-1B₁₋₂₉₈ (0.15 mM) and inhibitor (1-2 mM) in a 10% D₂O/90% H₂O Bis-Tris-d₁₉ buffer (50 mM, pH=6.5) solution containing NaCl (50 mM), DL-1,4-Dithiothreitol-d₁₀ (5 mM) and Sodium azide (0.02%).

The ¹H-¹⁵N HSQC NMR spectra are recorded at 20° C., on Bruker DRX500 or DMX600 NMR spectrometers. In all NMR experiments, pulsed field gradients are applied to afford the suppression of solvent signal. Quadrature detection in the indirectly detected dimensions is accomplished by using the States-TPPI method. The data are processed using Bruker software and analyzed using NMRCompass software (MSI) on Silicon Graphics computers.

### Analytical Methods for Measuring Inhibitor Activity on Cardiomyocytes

To determine if a PTP inhibitor is useful for promoting physiological cardiac hypertrophy (or similarly, treating pathological cardiac hypertrophy), the following in vitro and animal model assays can be used.

### Tissue Culture

Neonatal rat ventricular myocytes To determine whether a particular PTP inhibitor acts directly on cardiomyocytes to promote eccentric cellular hypertrophy, a cell culture system is needed. Cultures of neonatal rat ventricular myocytes (NVRM) often serve this purpose and are well known in the art.

Neonatal ventricular myocytes can be prepared from 2- to 3-day-old neonatal Wistar rats or other rats such as Sprague-Dawley (available from Charles River Laboratoreis, USA and other vendors). The rats are anesthetized and sacrificed by immersion in 70% (v/v) alcohol. The ventricles are removed and washed three times in Hank's solution, then minced and incubated with 0.25% (w/v) trypsinase for 10 min at 37° C. Addition of an equal volume of RPMI-1640 containing 10% (v/v) fetal bovine serum is used to terminate the digestion. The supernatant is discarded. Then, cells are incubated with fresh 0.25% trypsinase for 20 min at 37° C., and the supernatant is collected. The latter digestion step can be repeated up to four times to remove nonmyocytes. Cells in the supernatant are isolated by centrifugation for 10 min at 200 g at room temperature in a bench-top centrifuge. Cells are re-suspended in RPMI-1640 and incubated at 37° C. in a humidified atmosphere containing 5% (v/v) CO₂. To facilitate screening assays, often the cultures are plated into 96-well microtiter plates.

To further confirm purity of the cardiomyocyte culture and to assess cardiomyocyte shape during a time course in which different compounds or additives are added to each culture, immunoconfocal fluorescence can be used to better visualize the cardiomyocytes. Cardiomyocytes are fixed and permeabilized with precold methanol (−20° C., 15 min). After washing with PBS, cells are incubated with 1:200 monoclonal anti-α-actinin (Sigma) in PBS with 1% BSA (room temperature, 1 hour). PBS-washed cells are subsequently incubated (room temperature, 30 min) with 1:400 Alexa Fluor® 488 labeled goat anti-mouse IgG (H+L) (Molecular Probes) and viewed with a confocal microscope. Typically, more than 98% of cells are α-actinin-positive 96 hours after NRVM isolation, confirming the dominance of myocytes in the culture.

With the NRVM cultures described immediately above, one can induce concentric cellular hypertrophy (as a surrogate for pathological cardiac hypertrophy) by adding agents such as phorbol 12-myristate 13-acetate (PMA) to the cultures about 24 hours after serum starvation. Other inducers of concentric cellular hypertrophy include prostaglandin F2a, phenylephrine, and thyroid hormone T3 (triiodothyronine). Having induced this undesirable state in NRVM cultures, a variety of compounds, such as putative PTP inhibitors, can be added to assess changes in cardiomyocyte morphology, e.g., using the immunoflourescence microscopy described above.

ANF secretion. Other than a morphology readout, the NRVM cultures described above can be used to assess whether a sensitive marker protein for pathological cardiac hypertrophy is secreted from the cardiomyocytes into the cell culture medium.

Atrial Natriuretic Factor (ANF) is sensitive marker of pathological cardiac hypertrophy. It is 126 amino acid in length and cleaved to produce a 28 amino acid C-terminal active peptide (ANP). Under normal physiological conditions, it is expressed in the atrium, while in cardiac disease conditions such as heart failure, it is strongly expressed in the ventricle as well. The quantity of ANF produced in a cardiomyocyte culture correlates with the degree of pathological cardiac hypertrophy in cardiomyocytes cultures.

A competition-ELISA is used to measure the concentration of ANF in supernatants from treated and untreated cardiac myocytes. Anti-rat ANF antibody is used to bind endogenous ANF in supernatants, then protein G-coated plates are used to capture and correctly orient the antibodies. Biotinylated ANF is added for competition with endogenous ANF. Streptavidin-HRP binds the biotin residues on the conjugated ANF and HRP reacts with TMB substrate to cause a color change (at 450 nm). Using a standard curve, ng/ml ANF is calculated from absorbance values, and dose-response curves are determined. As previously mentioned, compounds such as PMA can induce secretion of ANF in these cultures. In addition, leukemia inhibitory factor (LIF) can also induce ANF secretion from cardiomyocytes.

ANF secretion in cardiomyocytes can determine whether a PTP inhibitor is useful in the methods of the invention. If a control culture of NRVM containing LIF exhibits high ANF levels in the supernatant, but in contrast the culture containing LIF and the PTP inhibitor contains lower levels of ANF, then the result indicates that the PTP inhibitor can be useful in the methods of the invention.

Commercial ELISA kits are available for testing samples for the presence of ANF. See, e.g., EIA Kit S-1132 sold by Bachem (King of Prussia, Pa., USA).

Cardiomyocyte Cytotoxicity. Cytotoxicity in cardiomyocytes can be measured indirectly, through assessing adenylate kinase activity in supernatants. When the cell membranes of cardiomyocytes rupture, this enzyme is among the components released. The amount of adenylate kinase enzyme activity measured in supernatants correlates with the amount of cytotoxicity of the cardiac myocytes. PTP inhibitors that result in low adenylate kinase activity in the supernatants, than compared to other PTP inhibitors, demonstrate desirable compounds for in vivo administration or testing.

Commercial assay kits for cytotoxicity based on detection of adenylate kinase activity are available. See, e.g., ToxiLight Bioassay Kit LT07-117 sold by Cambrex (USA).

### In Vivo Testing in Animal Models of Pathological Cardiac Hypertrophy

A variety of small animal models characterized by pathological cardiac hypertrophy are available to the skilled artisan, including genetic and nongenetic models. In addition, the manner in which a nongenetic animal model is prepared can be applied to an existing animal model of heart failure exhibiting pathological cardiac hypertrophy, or to an existing animal model of related disorders, such as diabetes or obesity, to combine disease states in one animal model. For a review of such animal models, see Wang et al., J Pharmcol Toxicol Methods 50:163-174, 2004. In each of the models described below, indeed for any model available to the skilled artisan, a candidate PTP inhibitor can be administered to the animal, compared to control, and the data analyzed to determine whether the PTP inhibitor promotes physiological cardiac hypertrophy. The particular details of such experimentation is well within the ken of the skilled artisan.

### Nongenetic Animal Models

Myocardial Infarction Coronary artery occlusion models are particularly relevant to heart failure and pathological cardiac hypertrophy. Such models have been developed in mice (see, e.g., Bayat et al., Bas Res Cardiol 97:206-213, 2002). To create coronary artery occlusion, the heart is exposed through athoracotomy, and the left coronary artery is ligated permanently under stereomicroscope with an 8-0 suture proximal to its main bifurcation. After closure of the chest, the animal is allowed to recover from surgery. Mice surviving beyond 2 weeks into the recovery period gradually develop heart failure and pathological cardiac hypertrophy. The infarct size in this model varies considerably, ranging from 10% to 45%, and this results in varied time courses for heart failure development. Mice with infarcts larger than 30% of the left ventricle usually present typical characteristics of heart failure with left ventricular dilation, pathological hypertrophy, impaired systolic function, and reduced exercise duration. In these mice, there are marked increases in the molecular markers for pathological hypertrophy and heart failure, such as the mRNA content of brain natriuretic peptide and atrial natriuretic factor (ANF) in the myocardium. However, these changes are minimal in mice with smaller infarcts. Age also affects the time course of heart failure development in coronary ligated mice. Two-month-old C57BL/6N mice tolerate coronary artery occlusion well without heart failure signs despite large infarcts after permanent coronary ligation, whereas 14-month-old mice develop heart failure, with only about 36% of animals surviving for more than 8 weeks.

Cardiac Pressure Overload Transverse aortic constriction (TAC) has been used to mechanically induce cardiac pressure overload. TAC is induced either by traditional thoractomy approach (see, e.g., Esposito et al., Circul 105:85-92, 2002) or by minimally invasive aortic banding through a small incision in the proximal sternum (Hu et al., Am J Physiol Heart Circul Physiol 285:H1261-H1269, 2003). Briefly, the transverse aorta between the right innominate and left common carotid arteries is dissected, and constriction is introduced by tying a nylon suture around the aorta and a blunted 27-gauge needle, which is then promptly removed. Alternatively, the abdominal aorta constriction between the diaphragm and renal arteries can be used to induce pressure overload. With either transverse or abdominal aortic constriction, the heart is challenged with an immediate pressure overload and pathological cardiac hypertrophy develops within days. In general, the extent of hypertrophy and the likelihood of progressing to heart failure increase the closer the constriction is to the heart. Following aortic constriction, there is an initial increase in cardiac contractility, with enhanced activity of the sympathetic nervous system, whereas 8 weeks after aortic constriction, progressive left ventricular enlargement and dysfunction are evident. As mentioned above, application of pressure overload in various genetic models can yield valuable information on the efficacy of candidate PTP inhibitors in different or combined disease contexts.

Cardiac Volume Overload An overt heart failure model induced by aortacaval shunt has been reported in mice (Scheuermann-Freestone et al., Eur J Heart Failure 3:535-543, 2001). In brief, through an abdominal midline incision, the vena cava as well as the abdominal aorta are dissected free of surrounding tissue above the renal arteries. After clamping temporarily the aorta proximal to the renal arteries, a disposable needle of 0.6 mm in outer diameter is used to puncture the aorta distal to the renal arteries. The needle is then advanced into the adjacent vena cava to connect both vessels. Afterwards, the needle is withdrawn, and the aortic puncture site is sealed with a drop of cyanoacrylate glue. After the clamp is removed, the patency of the aortacaval shunt is visually verified by swelling and mixing of venous and arterial blood in the vena cava. Four weeks after shunt induction, significant pathological cardiac hypertrophy develops with compromised left ventricular contractility and increased end-diastolic pressure. This model offers a relatively simple and rapid way of inducing heart failure with no need of thoractomy. As with cardiac pressure overload models, application of volume overload in various gene-manipulated mice can yield valuable information on the efficacy of candidate PTP inhibitors in different or combined disease contexts.

### Genetic Animal Models

A large number of pathological cardiac hypertrophy and heart failure models, created either by gene knockout or by overexpression, have been reported in genetically engineered mice. The major genes involved in these models can be generally categorized into those related to cytoskeletal or sarcomeric proteins, those related to neurohumoral receptors, those related to cell signalling proteins, those related to calcium (Ca2+)-regulating proteins, and those related to extracellular matrix (ECM) proteins. In addition, certain animal models may contain many genetic defects, the nature of which are not always fully understood, that lead to a predisposition to pathological cardiac hypertrophy or a disease that causes pathological cardiac hypertrophy. For the sake of brevity, only some models of some types will be described below.

Genetic Models Based on Cytoskeletal and Sarcomeric Proteins Cytoskeletal proteins play an important role in the maintenance of the cytoarchitectural organization and cardiac mechanotransduction. Knockout of the cytoskeletal muscle LIM protein (MLP) results in severe cardiac dilation, pathological cardiac hypertrophy and fibrosis, reduced cardiac performance, and blunted responsiveness to β-agonist stimulation, typical characteristics of human cardiomyopathy and heart failure (Arber et al., Cell 88:393-403, 1997). These knockout mice survive close to a normal life span, thus making this model suitable for long-term observation for PTP inhibitor effects on cardiac function and remodeling. The duration of the model also suggests utility for assessing prevention of pathological cardiac hypertrophy or related disease via promotion of physiological cardiac hypertrophy. Sarcomeric proteins are essential components of the contractile apparatus of the myocyte. Numerous mutations of sarcomeric proteins are known to be linked to familial hypertrophic cardiomyopathy (FHC), an inherited autosomal dominant disease. Phenotypes of FHC have been reproduced in transgenic mice expressing mutant genes encoding various sarcomeric proteins including β-myosin heavy chain (β-MHC), troponin T, and myosin-binding protein C. Mutations in several cardiac sarcomeric proteins, including actin, β-MHC, and troponin T, have also been identified as causes of heritable dilated cardiomyopathy, and therefore may be the foundation of additional transgenic models based on cytoskeletal or sarcomeric proteins.

Dahl-Salt Sensitive(SS) Rat When fed a high-salt diet, Dahl-SS rats develop hypertension and eventual decompensated, pressure-overload pathological cardiac hypertrophy. Dahl-SS rats are a well described animal model that is used to assess the impact of pharmacologic treatments for heart failure. Consequently, this model is particularly useful for the screening of candidate PTP inhibitors to assess promotion of physiological cardiac hypertrophy. To induce a disease state, the rats are fed a diet rich in salt, where the percentage of NaCl is chosen based upon its known effects on heart pathogenesis in the Dahl-SS rat, as originally described in Kong et al., Physiol Genomics 21:34-42, 2005. The salt can be added daily by addition directly to, e.g., a suspended food hopper, and mixed carefully with the daily food ration. In this fashion, the weight of the salt will not dilute out the other nutrients by weight, and sodium intake can be held equal between experimental groups. Rats are generally housed in 12-h light: 12-h dark cycle conditions, and water is provided ad libitum. For additional details on experimental set-ups using this model, see, e.g., Seymour et al., J Mol Cell Cardiol 41:661-668, 2006.

Spontaneously Hypertensive Heart Failure (SHHF) A relatively new genetic model of hypertension-induced heart failure and pathological cardiac hypertrophy, the spontaneously hypertensive heart failure (SHHF/Mcc-facp) rat is now commercially available (Genetic Models, Inc., Indianapolis, Ind., USA). This model is derived from a cross between spontaneously hypertensive rats (SHR) and Koletsky obese rats, and then bred to SHR. These rats exhibit early-onset hypertension, and all animals develop heart failure and pathological cardiac hypertrophy. While in heart failure, the SHHF rats exhibit numerous symptoms and biochemical changes that parallel documented changes in patients with hypertension, cardiomyopathy, and heart failure. Evaluation of SHHF animals at different ages can facilitate testing PTP inhibitor therapy at different ages. Use of the SHHF model is illustrated by the experimental design, for example, in Anderson et al., Hyperten 33:402-407, 1999.

Zucker Diabetic Fatty Rats The obese Zucker diabetic fatty (ZDF) male rat is a commonly used genetic model of type 2 diabetes and not strictly a disease model for pathological cardiac hypertrophy. However, since heart failure and pathological cardiac hypertrophy often occur in diabetic patients, ZDF rats can be combined with any of the nongenetic models discussed above or known to the skilled artisan. For example, the procedure for producing the cardiac pressure overload model described above can be performed on ZDF rats, thereby making an animal model that exhibits pathological cardiac hypertrophy in a diabetic background. The ZDF rat develops obesity due to the expression of the autosomal recessive fa gene resulting from a missense mutation in the extracellular domain of the leptin receptor. The ZDF rat is characterized by obesity, hyperglycaemia, hyperinsulinaemia, insulin resistance and hyperlipidaemia (Peterson et al., ILAR News 32:16-19, 1990). In contrast, heterozygous fa/+animals are lean, have functional leptin receptors, and normal glucose, insulin and lipid profiles. Although female ZDF rats have degrees of obesity and levels of insulin resistance comparable with male ZDF rats, they develop only hyperglycaemia when administered a diabetogenic diet (Corsetti et al., Atherosclerosis 148:231-241, 2000). An example of a diabetogenic diet is C13004 (Purina 5015 with 5% maltodextrin and 30% Merric 7-60 containing 12% protein, 48% fat, 40% carbohydrates), which is commercially available from Research Diets (New Brunswick, N.J.). For further details regarding a sample experimental set up for ZDF rats, see Schrijvers et al., Nephrol dial Transplant 21:324-329, 2006.

db/db Mice As described for the ZDF rats, this diabetes and obesity model can be used as a background for pathological cardiac hypertrophy via, for example, the cardiac pressure overload model. The leptin receptor-deficient db/db mouse is a well-established model of type II diabetes. In an isolated working heart setup (11 mM glucose, 0.7 mM FA, no insulin), hearts of these mice develop reduced cardiac mechanical performance, with an increase in left ventricular (LV) end-diastolic pressure, decreased LV developed pressure, and reductions in both cardiac output and cardiac power (Belke et al., Am J Physiol Endocrinol Metab 279:E1104-1113, 2000), even without additional insults such as TAC. For further details regarding a sample experimental set up for db/db mice, see Van den Bergh et al., Eur J Heart Failure 8:777-783, 2006.

Any of these animal models, once established, provides an in vivo system for testing the PTP inhibitors useful in the methods of the invention, including the molecules described below. For example, after establishment of the heart disease, the inhibitor can be given to the animal orally or parenterally, depending on the physical properties of the compounds, for a specified period of time, during or after which various biochemical and physiological markers can be assessed. See, for example, the diagnosis of heart failure in humans as discussed below. Sample-non-invasive imaging technologies such as echocardiography or magnetic resonance imaging are especially useful for monitoring the disease process in these animals.

Unlike human patients, the advantage of these animal models is that at the end of the experiment the animals can be sacrificed, and the heart tissue retrieved for histology. Using standard histological staining techniques, one can determine the extent of pathological or physiological hypertrophy exhibited in PTP inhibitor-treated versus untreated animals.

### Diagnosis of Heart Failure and Pathological Cardiac Hypertrophy

Doctors usually suspect heart failure, and consequently pathological cardiac hypertrophy, on the basis of symptoms alone. The diagnosis is supported by the results of a physical examination, including a weak, often rapid pulse, reduced blood pressure, abnormal heart sounds and fluid accumulation in the lungs (both heard through a stethoscope), an enlarged heart, swollen neck veins, an enlarged liver, and swelling in the abdomen or legs. A chest x-ray can show an enlarged heart and fluid accumulation in the lungs.

Procedures to evaluate heart function are usually performed. Electrocardiography (ECG) is almost always performed to determine whether the heart rhythm is normal, whether the walls of the ventricles are thickened, and whether the person has had a heart attack.

Echocardiography, which uses sound waves to produce an image of the heart, is one of the best procedures for evaluating heart function, including the pumping ability of the heart and the functioning of heart valves. It can show whether the heart walls are thickened, whether the valves are functioning normally, whether contractions are normal, and whether any area of the heart is contracting abnormally. Echocardiography may help determine whether heart failure is due to systolic or diastolic dysfunction by enabling doctors to estimate the thickness of the heart walls and the ejection fraction. The ejection fraction, an important measure of heart function, is the percentage of blood pumped out by the heart with each beat. A normal left ventricle ejects about 60% of the blood in it. If the ejection fraction is low, systolic dysfunction is likely; if it is normal or high, diastolic dysfunction is likely.

Other procedures, such as radionuclide imaging and cardiac catheterization with angiography, may be performed to identify the cause of heart failure. Rarely, a biopsy is needed, usually when doctors suspect infiltration of the heart (as occurs in amyloidosis) or myocarditis due to a bacterial, viral, or other infection. However, a biopsy may help determine definitely that the patient exhibits pathological cardiac hypetrophy.

### Specific Examples of PTP Inhibitors

The methods of the invention can be practiced with 1,1-dioxo-1,2,5-thiadiazolidin-3-one derivatives as described in the categories below.

### Category 1 PTP Inhibitors

The methods of the present invention can be practiced with the compounds of the formula

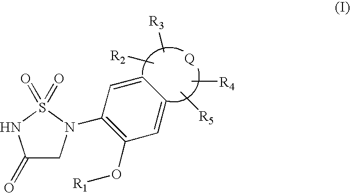

wherein

Q combined together with the carbon atoms to which it is attached form an aromatic, or a partially or fully saturated nonaromatic 5- to 8-membered carbocyclic or heterocyclic ring;

R₁ is hydrogen, —C(O)R₆, —C(O)NR₇R₈ or —C(O)OR₉ in which

R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 3- to 7-membered fused ring; or

sR₂ and R₃ combined are alkylene which together with the carbon atom to which they are attached form a 3- to 7-membered spirocyclic ring;

or a pharmaceutically acceptable salt thereof.

Preferred are compounds in the A group having the formula

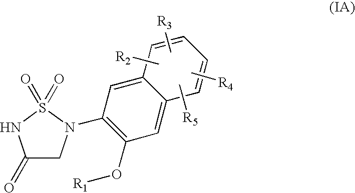

wherein
R₁ is hydrogen, —C(O)R₆, —C(O)NR₇R₈ or —C(O)OR₉ in which R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl; heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 5- to 7-membered fused ring; or
   or a pharmaceutically acceptable salt thereof.

Preferred are compounds of formula (IA) wherein
R₄ and R₅ are hydrogen;
   or a pharmaceutically acceptable salt thereof.

Further preferred are compounds of formula (IA) having the formula

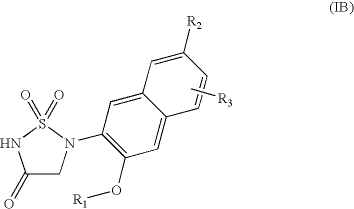

wherein
R₁ is hydrogen, —C(O)R₆, —C(O)NR₇R₈ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ and R₃ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Preferred are compounds of formula (IB) wherein
R₂ is —Y—(CH₂)ₙ—CR₁₀R₁₁—(CH₂)ₘ—X in which
   Y is oxygen or S(O)_{q} in which q is zero or an integer of 1 or 2; or
   Y is trans CH═CH; or
   Y is absent;
   n is an integer from 1 to 6;
   R₁₀ and R₁₁ are, independently from each other, hydrogen or lower alkyl; or
      R₁₀ and R₁₁ combined are alkylene which together with the carbon atom to which they are attached form a 3- to 7-membered ring;
m is zero or an integer of 1 or 2;
   X is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Further preferred are compounds of formula (IB) wherein
R₃ is hydrogen;
   or a pharmaceutically acceptable salt thereof.

Further preferred are also compounds of formula (IB) wherein
n is an integer of 2 or 3;
R₁₀ and R₁₁ are, independently from each other, hydrogen or lower alkyl;
m is zero or 1;
   X is hydroxy, carbamoyl, cyano, trifluoromethyl, free or esterified carboxy, monocyclic aryl or heterocyclyl;
   or a pharmaceutically acceptable salt thereof.

More preferred are compounds of formula (IB) wherein
Y is absent;
   or a pharmaceutically acceptable salt thereof.

Even more preferred are compounds of formula (IB) wherein
n is 3;
R₁₀ and R₁₁ are lower alkyl;
m is zero or 1;
X is hydroxy, cyano or free or esterified carboxy;
   or a pharmaceutically acceptable salt thereof.

Most preferred are compounds of formula (IB) wherein
R₁₀ and R₁₁ are methyl;
   or a pharmaceutically acceptable salt thereof.

Especially preferred are compounds of formula (IB) wherein
R₁ is hydrogen or —C(O)R₆ in which R₆ is monocyclic aryl;
   or a pharmaceutically acceptable salt thereof.

Preferred are also compounds in the A group having the formula

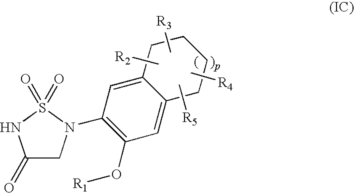

wherein
R₁ is hydrogen or —C(O)R₆, —C(O)NR₇R₈ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 3- to 7-membered fused ring; or
   R₂ and R₃ combined are alkylene which together with the carbon atom to which they are attached form a 3- to 7-membered spirocyclic ring;
   p is zero or 1;
   or a pharmaceutically acceptable salt thereof.

Preferred are compounds of formula (IC) wherein
R₄ and R₅ are hydrogen;
   or a pharmaceutically acceptable salt thereof.

Preferred are also compounds of formula (IC) wherein

R₂ and R₃ are, independently from each other, hydrogen, halogen or (C₁₋₄)alkyl optionally substituted by at least one halogen;
or a pharmaceutically acceptable salt thereof.

Preferred are also compounds of formula (IC) wherein
p is 1;
   or a pharmaceutically acceptable salt thereof.

Further preferred are compounds of formula (IC) having the formula

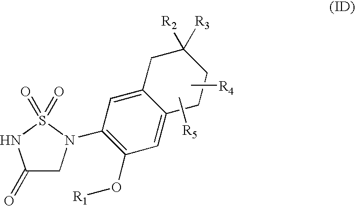

wherein
R₁ is hydrogen or —C(O)R₆, —C(O)NR₇R₈ or —C(O)OR₉ in which
   R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the carbon atom to which they are attached form a 3- to 7-membered spirocyclic ring;
   or a pharmaceutically acceptable salt thereof.

Preferred are compounds of formula (ID) wherein
R₄ and R₅ are hydrogen;
   or a pharmaceutically acceptable salt thereof.

Preferred are also compounds of formula (ID), designated as the B group, wherein

R₂ and R₃ are, independently from each other, hydrogen, halogen or (C₁₋₄)alkyl optionally substituted by at least one halogen;
or a pharmaceutically acceptable salt thereof.

Preferred are compounds in the B group wherein
R₁ is hydrogen or —C(O)R₆ in which R₆ is monocyclic aryl;
   or a pharmaceutically acceptable salt thereof.

Preferred are also compounds of formula (ID), designated as the C group, wherein

R₂ and R₃ combined are alkylene which together with the carbon atom to which they are attached form a 3- to 5-membered spirocyclic ring;
or a pharmaceutically acceptable salt thereof.

Preferred are compounds in the C group, wherein
R₂ is hydrogen or —C(O)R₆ in which R₆ is monocyclic aryl;
   or a pharmaceutically acceptable salt thereof.

Preferred are also compounds of formula (ID), designated as the D group, wherein
R₂ is —Y—(CH₂)ₙ—CR₁₀R₁₁—(CH₂)ₘ—X in which
   Y is oxygen or S(O)_{q} in which q is zero or an integer of 1 or 2; or
   Y is trans CH═CH; or
   Y is absent;
   n is an integer from 1 to 6;
   R₁₀ and R₁₁ are, independently from each other, hydrogen or lower alkyl; or
      R₁₀ and R₁₁ combined are alkylene which together with the carbon atom to which they are attached form a 3- to 7-membered ring;
m is zero or an integer of 1 or 2,
   X is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Preferred are compounds in the D group wherein
R₃ is hydrogen;
   or a pharmaceutically acceptable salt thereof.

Further preferred are compounds in the D group wherein
n is an integer of 2 or 3;
R₁₀ and R₁₁ are, independently from each other, hydrogen or lower alkyl;
m is zero or 1;
X is hydroxy, carbamoyl, cyano, trifluoromethyl, free or esterified carboxy, monocyclic aryl or heterocyclyl;
   or a pharmaceutically acceptable salt thereof.

More preferred are compounds in the D group wherein
Y is absent;
   or a pharmaceutically acceptable salt thereof.

Even more preferred are compounds in the D group wherein
n is 3;
R₁₀ and R₁₁ are lower alkyl;
m is zero or 1;
X is hydroxy, cyano or free or esterified carboxy;
   or a pharmaceutically acceptable salt thereof.

Most preferred are compounds in the D group wherein
R₁₀ and R₁₁ are methyl;
   or a pharmaceutically acceptable salt thereof.

Especially preferred are compounds in the D group wherein
R₁ is hydrogen or —C(O)R₆ in which R₆ is monocyclic aryl;
   or a pharmaceutically acceptable salt thereof.

Particular embodiments are:
5-(3,6-Dihydroxynaphthalen-2-yl)-1,1-dioxo-1,2,5-thiadiazolidin-3-one;
5-(3,7-Dihydroxynaphthalen-2-yl)-1,1-dioxo-1,2,5-thiadiazolidin-3-one potassium salt;
5-(7-Bromo-3-hydroxynaphthalen-2-yl)-1,1-dioxo-1,2,5-thiadiazolidin-3-one;
5-(7-Ethyl-3-hydroxynaphthalen-2-yl)-1,1-dioxo-1,2,5-thiadiazolidin-3-one;
5-{3-Hydroxy-7-[2-(4-methoxyphenyl)-ethyl]-naphthalen-2-yl}-1,1-dioxo-1,2,5-thiadiazolidin-3-one;
5-{3-Hydroxy-7-[2-(4-trifluoromethylphenyl)-ethyl]-naphthalen-2-yl}-1,1-dioxo-1,2,5-thiadiazolidin-3-one;
5-{3-Hydroxy-7-[2-(3-methoxyphenyl)-ethyl]-naphthalen-2-yl}-1,1-dioxo-1,2,5-thiadiazolidin-3-one;
5-[3-Hydroxy-7-(4-methylpentyl)-naphthalen-2-yl]-1,1-dioxo-1,2,5-thiadiazolidin-3-one;
{3-[6-Hydroxy-7-(1,1,4-trioxo-1,2,5-thiadiazolidin-2-yl)naphthalen-2-yl]-phenyl}-acetic acid;
5-(3-Hydroxy-7-phenylnaphthalen-2-yl)-1,1-dioxo-1,2,5-thiadiazolidin-3-one;
3-[6-Hydroxy-7-(1,1,4-trioxo-1,2,5-thiadiazolidin-2-yl)-naphthalen-2-yl]-benzoic acid;
5-[3-Hydroxy-7-(3-trifluoromethoxyphenyl)-naphthalen-2-yl]-1,1-dioxo-1,2,5-thiadiazolidin-3-one;
{3-[6-Hydroxy-7-(1,1,4-trioxo-1,2,5-thiadiazolidin-2-yl)-naphthalen-2-yl]-phenyl}acetonitrile;
5-[3-Hydroxy-7-(3-hydroxymethylphenyl)-naphthalen-2-yl]-1,1-dioxo-1,2,5-thiadiazolidin-3-one;
3-{3-[6-Hydroxy-7-(1,1,4-trioxo-thiadiazolidin-2-yl)-naphthalen-2yl]-phenyl}-propionic acid;
6-Hydroxy-7-(1,1,4-trioxo-1,2,5-thiadiazolidin-2-yl)-naphthalene-2-carbonitrile;
3-[6-Hydroxy-7-(1,1,4-trioxo-1,2,5-thiadiazolidin-2-yl)-naphthalen-2-yl]-benzonitrile;
5-[7-(3,3-Dimethylbutyl)-3-hydroxynaphthalen-2-yl]-1,1-dioxo-1,2,5-thiadiazolidin-3-one;
5-[3-Hydroxy-7-(3-trifluoromethylphenyl)-naphthalen-2-yl]-1,1-dioxo-1,2,5-thiadiazolidin-3-one;
3-[6-Hydroxy-7-(1,1,4-trioxo-1,2,5-thiadiazolidin-2-yl)-naphthalen-2-yl]-benzoic acid ethyl ester;
5-[3-Hydroxy-7-(3-methanesulfonylphenyl)-naphthalen-2-yl]-1,1-dioxo-1,2,5-thiadiazolidin-3-one;
3-{3-[6-Hydroxy-7-(1,1,4-trioxo-1,2,5-thiadiazolidin-2-yl)-naphthalen-2-yl]-phenyl}-propionitrile;
5-[3-Hydroxy-7-(3-methoxymethylphenyl)-naphthalen-2-yl]-1,1-dioxo-1,2,5-thiadiazolidin-3-one;
5-(7-Furan-3-yl-3-hydroxynaphthalen-2-yl)-1,1-dioxo-1,2,5-thiadiazolidin-3-one;
N-{3-[6-Hydroxy-7-(1,1,4-trioxo-1,2,5-thiadiazolidin-2-yl)-naphthalen-2-yl]-phenyl}-methanesulfonamide;
5-[7-(2-Fluorophenyl)-3-hydroxynaphthalen-2-yl]-1,1-dioxo-1,2,5-thiadiazolidin-3-one;
5-(3-Hydroxy-7-o-tolylnaphthalen-2-yl)-1,1-dioxo-1,2,5-thiadiazolidin-3-one;
5-(3-Hydroxy-7-pentylnaphthalen-2-yl)-1,1-dioxo-1,2,5-thiadiazolidin-3-one;
5-(3-Hydroxy-7-propylnaphthalen-2-yl)-1,1-dioxo-1,2,5-thiadiazolidin-3-one;
5-[3-Hydroxy-7-(tetrahydrofuran-3-yl)-naphthalen-2-yl]-1,1-dioxo-1,2,5-thiadiazolidin-3-one;
{3-[6-Hydroxy-7-(1,1,4-trioxo-1,2,5-thiadiazolidin-2-yl)-naphthalen-2-yl]-phenyl}-acetic acid ethyl ester;
3-{3-[6-Hydroxy-7-(1,1,4-trioxo-1,2,5-thiadiazolidin-2-yl)-naphthalen-2-yl]-phenyl}-propionic acid ethyl ester;
5-[6-Hydroxy-7-(1,1,4-trioxo-1,2,5-thiadiazolidin-2-yl)-naphthalen-2-yl]-pentanoic acid ethyl ester;
4-[6-Hydroxy-7-(1,1,4-trioxo-1,2,5-thiadiazolidin-2-yl)-naphthalen-2-yl]-2,2-dimethyl-butyric acid;
5-[3-Hydroxy-7-((S)-4-hydroxypentyl)-naphthalen-2-yl]-1,1-dioxo-1,2,5-thiadiazolidin-3-one;
4-[6-Hydroxy-7-(1,1,4-trioxo-1,2,5-thiadiazolidin-2-yl)-naphthalen-2-yl]-2-methylbutyronitrile;
5-[6-Hydroxy-7-(1,1,4-trioxo-1,2,5-thiadiazolidin-2-yl)-naphthalen-2-yl]-2-methylpentanoic acid ethyl ester;
5-[3-Hydroxy-7-(3-methylbutyl)-naphthalen-2-yl]-1,1-dioxo-1,2,5-thiadiazolidin-3-one;
5-[6-Hydroxy-7-(1,1,4-trioxo-1,2,5-thiadiazolidin-2-yl)-naphthalen-2-yl]-2,2-dimethylpentanenitrile;
5-[6-Hydroxy-7-(1,1,4-trioxo-1,2,5-thiadiazolidin-2-yl)-naphthalen-2-yl]-pentanoic acid;
5-[3-Hydroxy-7-(5-hydroxypentyl)-naphthalen-2-yl]-1,1-dioxo-1,2,5-thiadiazolidin-3-one;
2-Hydroxy-6-{2-[6-hydroxy-7-(1,1,4-trioxo-1,2,5-thiadiazolidin-2-yl)-naphthalen-2-yloxy]-ethoxy}-N,N-dimethylbenzamide;
2-Hydroxy-6-{4-[6-hydroxy-7-(1,1,4-trioxo-1,2,5-thiadiazolidin-2-yl)-naphthalen-2-yl]-butoxy}-N,N-dimethylbenzamide;
5-{3-Hydroxy-7-[3-(2-hydroxyethoxy)-propyl]-naphthalen-2-yl}-1,1-dioxo-1,2,5-thiadiazolidin-3-one;
5-{3-Hydroxy-7-[2-(2-methoxyphenyl)-ethyl]-naphthalen-2-yl}-1,1-dioxo-1,2,5-thiadiazolidin-3-one;
5-[3-Hydroxy-7-(5-oxohexyl)-naphthalen-2-yl]-1,1-dioxo-1,2,5-thiadiazolidin-3-one;
5-{7-[3-(3,5-Dimethylpyrazol-1-yl)-propyl]-3-hydroxy-naphthalen-2-yl}-1,1-dioxo-1,2,5-thiadiazolidin-3-one;
5-{3-Hydroxy-7-[3-(2-oxocyclohexyl)-propyl]-naphthalen-2-yl}-1,1-dioxo-1,2,5-thiadiazolidin-3-one;
5-{3-Hydroxy-7-[4-hydroxy-4-(tetrahydrofuran-2-yl)-butyl]-naphthalen-2-yl}-1,1-dioxo-1,2,5-thiadiazolidin-3-one;
5-{3-Hydroxy-7-[1-(2-oxopyrrolidin-1-yl)-ethyl]naphthalen-2-yl}-1,1-dioxo-1,2,5-thiadiazolidin-3-one;
5-[3-Hydroxy-7-(3-phenylpropyl)-naphthalen-2-yl]-1,1-dioxo-1,2,5-thiadiazolidin-3-one;
5-[3-Hydroxy-7-(3-pentafluorophenylpropyl)-naphthalen-2-yl]-1,1-dioxo-1,2,5-thiadiazolidin-3-one;
2-{3-[6-Hydroxy-7-(1,1,4-trioxo-1,2,5-thiadiazolidin-2-yl)-naphthalen-2-yl]-propyl}benzonitrile;
5-[3-Hydroxy-7-((R)-4-hydroxypentyl)-naphthalen-2-yl]-1,1-dioxo-1,2,5-thiadiazolidin-3-one;
5-[3-Hydroxy-7-(4-hydroxypentyl)-naphthalen-2-yl]-1,1-dioxo-1,2,5-thiadiazolidin-3-one;
5-[3-Hydroxy-7-(4-hydroxy-3-methylbutyl)-naphthalen-2-yl]-1,1-dioxo-1,2,5-thiadiazolidin-3-one;
5-[7-(4-Ethyl-4-hydroxyhexyl)-3-hydroxynaphthalen-2-yl]-1,1-dioxo-1,2,5-thiadiazolidin-3-one;
5-[3-Hydroxy-7-(4-hydroxyheptyl)-naphthalen-2-yl]-1,1-dioxo-1,2,5-thiadiazolidin-3-one;
5-{3-Hydroxy-7-[3-(1-hydroxycyclohexyl)-propyl]-naphthalen-2-yl}-1,1-1,2,5-thiadiazolidin-3-one;
5-[6-Hydroxy-7-(1,1,4-trioxo-1,2,5-thiadiazolidin-2-yl)-naphthalen-2-yl]-2,2-dimethylpentanoic acid;
5-{3-Hydroxy-7-[2-((1S,2R)-2-hydroxycyclopentyl)-ethyl]-naphthalen-2-yl}-1,1-dioxo-1,2,5-thiadiazolidin-3-one;
5-[6-Hydroxy-7-(1,1,4-trioxo-1,2,5-thiadiazolidin-2-yl)-naphthalen-2-yl]-pentanenitrile;
5-{3-Hydroxy-7-[3-(2-hydroxycyclohexyl)-propyl]-naphthalen-2-yl}-1,1-dioxo-1,2,5-thiadiazolidin-3-one;
5-[6-Hydroxy-7-(1,1,4-trioxo-1,2,5-thiadiazolidin-2-yl)-naphthalen-2-yl]-2,2-dimethylpentanoic acid methyl ester;
5-[3-Hydroxy-7-(5,5,5-trifluoro-4-hydroxy-4-methylpentyl)-naphthalen-2-yl]-1,1-dioxo-1,2,5-thiadiazolidin-3-one;
Acetic acid 4-[6-hydroxy-7-(1,1,4-trioxo-1,2,5-thiadiazolidin-2-yl)-naphthalen-2-yl]-2-methyl butyl ester;
5-[3-Hydroxy-7-(5,5,5-trifluoro-4-hydroxypentyl)-naphthalen-2-yl]-1,1-dioxo-1,2,5-thiadiazolidin-3-one;
5-[3-Hydroxy-7-(4-hydroxy-4-methylpentyl)-naphthalen-2-yl]-1,1-dioxo-1,2,5-thiadiazolidin-3-one;
5-(7-Cyclopentyl-3-hydroxynaphthalen-2-yl)-1,1-dioxo-1,2,5-thiadiazolidin-3-one;
5-(7-Cyclohexyl-3-hydroxynaphthalen-2-yl)-1,1-dioxo-1,2,5-thiadiazolidin-3-one;
5-[3-Hydroxy-7-(3-methylsulfanylphenyl)-naphthalen-2-yl]-1,1-dioxo-1,2,5-thiadiazolidin-3-one;
5-[3-Hydroxy-7-((E)-4-hydroxy-4-methylpent-1-enyl)-naphthalen-2-yl]-1,1-dioxo-1,2,5-thiadiazolidin-3-one;
5-[6-Hydroxy-7-(1,1,4-trioxo-1,2,5-thiadiazolidin-2-yl)-naphthalen-2-yl]-thiophene-2-carbonitrile;
{3-[6-Hydroxy-7-(1,1,4-trioxo-1,2,5-thiadiazolidin-2-yl)-naphthalen-2-yl]-benzyl}-carbamic acid methyl ester;
(E)-5-[6-Hydroxy-7-(1,1,4-trioxo-1,2,5-thiadiazolidin-2-yl)-naphthalen-2-yl]-pent-4-enenitrile;
(E)-5-[6-Hydroxy-7-(1,1,4-trioxo-1,2,5-thiadiazolidin-2-yl)-naphthalen-2-yl]-2-methylpent-4-enoic acid ethyl ester;
(E)-5-[6-Hydroxy-7-(1,1,4-trioxo-1,2,5-thiadiazolidin-2-yl)naphthalen-2-yl]-2-methylpent-4-enoic acid;
(E)-5-[6-Hydroxy-7-(1,1,4-trioxo-1,2,5-thiadiazolidin-2-yl)-naphthalen-2-yl]-pent-4-enoic acid;
5-[6-Hydroxy-7-(1,1,4-trioxo-1,2,5-thiadiazolidin-2-yl)-naphthalen-2-yl]-pentanoic acid isopropyl ester;
5-[6-Hydroxy-7-(1,1,4-trioxo-1,2,5-thiadiazolidin-2-yl)-naphthalen-2-yl]-2-methylpentanoic acid methyl ester;
5-[6-Hydroxy-7-(1,1,4-trioxo-1,2,5-thiadiazolidin-2-yl)-naphthalen-2-yl]-2-methylpentanoic acid;
5-[7-(4,5-Dihydroxy-4,5-dimethylhex-1-enyl)-3-hydroxynaphthalen-2-yl]-1,1-dioxo-1,2,5-thiadiazolidin-3-one;
5-[7-(4,5-Dihydroxy-4,5-dimethylhexyl)-3-hydroxynaphthalen-2-yl]-1,1-dioxo-1,2,5-thiadiazolidin-3-one;
5-[7-(4,4-Dimethylpentyl)-3-hydroxynaphthalen-2-yl]-1,1-dioxo-1,2,5-thiadiazolidin-3-one;
Benzoic acid 6-(3-cyano-3-methylpropyl)-3-(1,1,4-trioxo-1,2,5-thiadiazolidin-2-yl)-naphthalen-2-yl ester;
2,2-Dimethylpropionic acid 6-(3-cyano-3-methylpropyl)-3-(1,1,4-trioxo-1,2,5-thiadiazolidin-2-yl)-naphthalen-2-yl ester;
Propionic acid 6-(3-cyano-3-methylpropyl)-3-(1,1,4-trioxo-1,2,5-thiadiazolidin-2-yl)-naphthalen-2-yl ester;
2-Ethylbutyric acid 6-(3-cyano-3-methylpropyl)-3-(1,1,4-trioxo-1,2,5-thiadiazolidin-2-yl)-naphthalen-2-yl ester;
Hexanoic acid 6-(3-cyano-3-methylpropyl)-3-(1,1,4-trioxo-1,2,5-thiadiazolidin-2-yl)-naphthalen-2-yl ester;
2-Acetoxy-benzoic acid 6-(3-cyano-3-methylpropyl)-3-(1,1,4-trioxo-1,2,5-thiadiazolidin-2-yl)-naphthalen-2-yl ester;
Pentanoic acid 6-(3-cyano-3-methylpropyl)-3-(1,1,4-trioxo-1,2,5-thiadiazolidin-2-yl)-naphthalen-2-yl ester;
Acetic acid 6-(3-cyano-3-methylpropyl)-3-(1,1,4-trioxo-1,2,5-thiadiazolidin-2-yl)-naphthalen-2-yl ester;
3-Methylbenzoic acid 6-(3-cyano-3-methylpropyl)-3-(1,1,4-trioxo-1,2,5-thiadiazolidin-2-yl)-naphthalen-2-yl ester;
2-Methylbenzoic acid 6-(3-cyano-3-methylpropyl)-3-(1,1,4-trioxo-1,2,5-thiadiazolidin-2-yl)-naphthalen-2-yl ester;
4-Butylbenzoic acid 6-(3-cyano-3-methylpropyl)-3-(1,1,4-trioxo-1,2,5-thiadiazolidin-2-yl)-naphthalen-2-yl ester;
Cyclohexanecarboxylic acid 6-(3-cyano-3-methylpropyl)-3-(1,1,4-trioxo-1,2,5-thiadiazolidin-2-yl)-naphthalen-2-yl ester;
4-tert-Butylbenzoic acid 6-(3-cyano-3-methylpropyl)-3-(1,1,4-trioxo-1,2,5-thiadiazolidin-2-yl)-naphthalen-2-yl ester;
2,2-Dimethylpropionic acid 6-(3-cyanophenyl)-3-(1,1,4-trioxo-1,2,5-thiadiazolidin-2-yl)-naphthalen-2-yl ester;
Benzoic acid 6-(4-ethoxycarbonylbutyl)-3-(1,1,4-trioxo-1,2,5-thiadiazolidin-2-yl)-naphthalen-2-yl ester;
Benzoic acid 6-(3-methylbutyl)-3-(1,1,4-trioxo-1,2,5-thiadiazolidin-2-yl)-naphthalen-2-yl ester; Benzoic acid 6-((E)-4-hydroxy-4-methylpent-1-enyl)-3-(1,1,4-trioxo-1,2,5-thiadiazolidin-2-yl)-naphthalen-2-yl ester;
Benzoic acid 6-methyl-3-(1,1,4-trioxo-1,2,5-thiadiazolidin-2-yl)-naphthalen-2-yl ester;
Benzoic acid 6-(5-hydroxy-4,4-dimethylpentyl)-3-(1,1,4-trioxo-1,2,5-thiadiazolidin-2-yl)-naphthalen-2-yl ester;
5-[3-Hydroxy-7-(5-hydroxy-4,4-dimethylpentyl)-naphthalen-2-yl]-1,1-dioxo-1,2,5-thiadiazolidin-3-one;
5-(3-Hyrdoxy-5,6,7,8-tetrahydronapthalen-2-yl)-1,1-dioxo-1,2,5-thiadiazolidin-3-one;
5-(3,6-Dihydroxy-5,6,7,8-tetrahydronaphthalen-2-yl)-1,1-dioxo-1,2,5-thiadiazolidin-3-one;
5-(3-Hydroxy-6-methoxy-5,6,7,8-tetrahydronaphthalen-2-yl)-1,1-dioxo-1,2,5-thiadiazolidin-3-one;
5-(6-Ethoxy-3-hydroxy-5,6,7,8-tetrahydronaphthalen-2-yl)-1,1-dioxo-1,2,5-thiadiazolidin-3-one;
5-(3-Hydroxy-7-methyl-5,6,7,8-tetrahydronaphthalen-2-yl)-1,1-dioxo-1,2,5-thiadiazolidin-3-one;
5-(3-Hydroxy-7,7-dimethyl-5,6,7,8-tetrahydronaphthalen-2-yl)-1,1-dioxo-1,2,5-thiadiazolidin-3-one;
5-(3-Hydroxy-7-trifluoromethyl-5,6,7,8-tetrahydronaphthalen-2-yl)-1,1-dioxo-1,2,5-thiadiazolidin-3-one;
5-(3-Hydroxy-7-isopropyl-5,6,7,8-tetrahydronaphthalen-2-yl)-1,1-dioxo-1,2,5-thiadiazolidin-3-one;
5-(7-Ethyl-3-hydroxy-5,6,7,8-tetrahydronaphthalen-2-yl)-1,1-dioxo-1,2,5-thiadiazolidin-3-one;
5-(7,7-Diethyl-3-hydroxy-5,6,7,8-tetrahydronaphthalen-2-yl)-1,1-dioxo-1,2,5-thiadiazolidin-3-one;
5-(3-Hydroxy-7,7-dipropyl-5,6,7,8-tetrahydronaphthalen-2-yl)-1,1-dioxo-1,2,5-thiadiazolidin-3-one;
5-(6′-Hydroxy-3′,4′-dihydro-1′H-spiro[cyclopentane-1,2′-naphthalen]-7′-yl) 1,2,5-thiadiazolidin-3-one 1,1-dioxide;
5-((S)-7-Ethyl-3-hydroxy-5,6,7,8-tetrahydronaphthalen-2-yl) 1,1-dioxo-1,2,5-thiadiazolidin-3-one;
5-[6-Hydroxy-7-(1,1,4-trioxo-1,2,5-thiadiazolidin-2-yl)-1,2,3,4-tetrahydronaphthalen-2-yl]-2,2-dimethylpentanoic acid methyl ester;
5-[6-Hydroxy-7-(1,1,4-trioxo-1,2,5-thiadiazolidin-2-yl)-1,2,3,4-tetrahydronaphthalen-2-yl]-2,2-dimethylpentanoic acid;
5-(6-Hydroxy-2-methyl-2,3-dihydrobenzo[b]thiophen-5-yl)-1,1-dioxo-1,2,5-thiadiazolidin-3-one;
5-(6-Hydroxyindan-5-yl)-1,1-dioxo-1,2,5-thiadiazolidin-3-one;
5-(6-Hydroxy-2,2-dimethylindan-5-yl)-1,1-dioxo-1,2,5-thiadiazolidin-3-one;
5-(6-Hydroxy-2-methylindan-5-yl)-1,1-dioxo-1,2,5-thiadiazolidin-3-one; Benzoic acid 6,6-dimethyl-3-(1,1,4-trioxo-1,2,5-thiadiazolidin-2-yl)-5,6,7,8-tetrahydronaphthalen-2-yl ester;
Benzoic acid (S)-6-ethyl-3-(1,1,4-trioxo-1,2,5-thiadiazolidin-2-yl)-5,6,7,8-tetrahydronaphthalen-2-yl ester;
Benzoic acid 6-ethyl-3-(1,1,4-trioxo-1,2,5-thiadiazolidin-2-yl)-5,6,7,8-tetrahydronaphthalen-2-yl ester;
Benzoic acid 6,6-diethyl-3-(1,1,4-trioxo-1,2,5-thiadiazolidin-2-yl)-5,6,7,8-tetrahydronaphthalen-2-yl ester;
Benzoic acid 2,2-dimethyl-6-(1,1,4-trioxo-1,2,5-thiadiazolidin-2-yl)-indan-5-yl ester;
5-(3-Allyloxy-6-hydroxybenzo[d]isoxazol-5-yl)-1,1-dioxo-1,2,5-thiadiazolidin-3-one;
5-Hydroxy-6-(1,1,4-trioxo-1,2,5-thiadiazolidin-2-yl)-1H-indole-2-carboxylic acid ethyl ester potassium salt;
5-Hydroxy-6-(1,1,4-trioxo-1,2,5-thiadiazolidin-2-yl)-1H-indole-2-carboxylic acid 3-methyl-butyl ester;
5-Hydroxy-6-(1,1,4-trioxo-1,2,5-thiadiazolidin-2-yl)-1H-indole-2-carboxylic acid isobutyl ester;
5-Hydroxy-6-(1,1,4-trioxo-1,2,5-thiadiazolidin-2-yl)-1H-indole-2-carboxylic acid; and
5-(7-Hydroxy-3-methoxy-2-oxo-2H-chromen-6-yl)-1,1-dioxo-1,2,5-thiadiazolidin-3-one;
5-(3-Hydroxy-7-methoxynaphthalen-2-yl)-1,1-dioxo-[1,2,5]thiadiazolidin-3-one;
5-(3-Hydroxy-7-methoxynaphthalen-2-yl)-1,1-dioxo-[1,2,5]thiadiazolidin-3-one potassium salt;
5-(3-Hydroxy-7-propoxynaphthalen-2-yl)-1,1-dioxo-[1,2,5]thiadiazolidin-3-one;
5-(3-Hydroxy-7-propoxynaphthalen-2-yl)-1,1-dioxo-[1,2,5]thiadiazolidin-3-one potassium salt;
5-(3-Hydroxynaphthalen-2-yl)-1,1-dioxo-[1,2,5]thiadiazolidin-3-one;
5-(3-Hydroxynaphthalen-2-yl)-1,1-dioxo-[1,2,5]thiadiazolidin-3-one potassium salt;
5-(3-Hydroxy-7-methyl-naphthalen-2-yl)-1,1-dioxo-1,2,5-thiadiazolidin-3-one; and
5-(3-Hydroxy-7-methyl-naphthalen-2-yl)-1,1-dioxo-1,2,5-thiadiazolidin-3-one potassium salt
   or a pharmaceutically acceptable salt thereof.

### Category 2 PTP Inhibitors

The methods of the invention can be practiced with the compounds of the formula

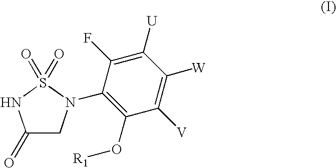

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   U, W and V are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, aryloxy, arylthio, heterocyclyl, heterocycloyloxy, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   U and W combined together with the carbon atoms to which they are attached form an optionally substituted aromatic, or a partially or fully saturated nonaromatic 5- to 8-membered carbocyclic or heterocyclic ring; or
   W and V combined together with the carbon atoms to which they are attached form an optionally substituted aromatic, or partially or fully saturated nonaromatic 5- to 8-membered carbocyclic or heterocyclic ring;
   or a pharmaceutically acceptable salt thereof.

Preferred are the compounds of formula (I) wherein

U and W combined together with the carbon atoms to which they are attached form an optionally substituted aromatic, or a partially or fully saturated nonaromatic 5- to 8-membered carbocyclic or heterocyclic ring;
V is hydrogen;
or a pharmaceutically acceptable salt thereof.

Further preferred are the compounds of formula (I) having the formula

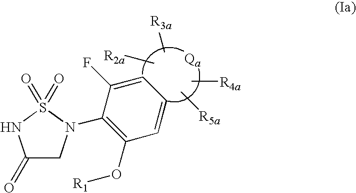

wherein
Qₐ combined together with the carbon atoms to which it is attached form an aromatic, or a partially or fully saturated nonaromatic 5- to 8-membered carbocyclic or heterocyclic ring;
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the ring atoms to which they are attached form a 3- to 7-membered fused ring; or
   R₂ₐ and R₃ₐ combined are alkylene which together with the carbon atom to which they are attached form a 3- to 7-membered spirocyclic ring;
   or a pharmaceutically acceptable salt thereof.

Preferred are compounds of formula (Ia), designated as the A group, wherein

Qₐ combined together with the carbon atoms to which it is attached form an aromatic, or a partially or fully saturated 5- to 6-membered carbocyclic ring;
or a pharmaceutically acceptable salt thereof.

Preferred are compounds in the A group having the formula

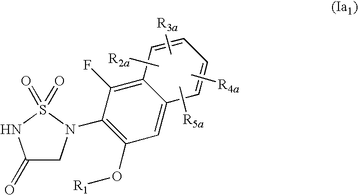

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the ring atoms to which they are attached form a 5- to 7-membered fused ring; or
   or a pharmaceutically acceptable salt thereof.

Preferred are compounds of formula (Ia₁) wherein
R₄ₐ and R₅ₐ are hydrogen;
   or a pharmaceutically acceptable salt thereof.

Further preferred are compounds of formula (Ia₁) having the formula

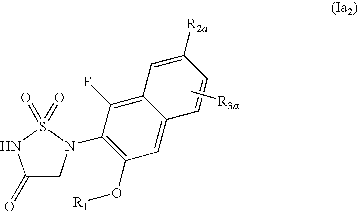

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ and R₃ₐ are, independently from each other, hydrogen, hydroxy, halogen, cyano, nitro, alkoxy, alkylthio, alkylthiono, sulfonyl, free or esterifled carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterifled carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Preferred are compounds of formula (Ia₂) wherein
R₂ₐ is —Yₐ—(CH₂)ₙ—CR₆ₐR₇ₐ—(CH₂)ₘ—Xₐ in which
   Yₐ is oxygen or S(O)_{q} in which q is zero or an integer of 1 or 2; or
   Yₐ is trans CH═CH; or
   Yₐ is absent;
   n is an integer from 1 to 6;
      R₆ₐ and R₇ₐ are, independently from each other, hydrogen or lower alkyl; or

R₆ₐ and R₇ₐ combined are alkylene which together with the carbon atom to which they are attached form a 3- to 7-membered ring;
m is zero or an integer of 1 or 2;
   Xₐ is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Further preferred are compounds of formula (Ia₂) wherein
R₃ₐ is hydrogen;
   or a pharmaceutically acceptable salt thereof.

Further preferred are also compounds of formula (Ia₂) wherein
n is an integer of 2 or 3;
R₆ₐ and R₇ₐ are, independently from each other, hydrogen or lower alkyl;
m is zero or 1;
   Xₐ is hydroxy, carbamoyl, cyano, trifluoromethyl, free or esterified carboxy, monocyclic aryl or heterocyclyl;
   or a pharmaceutically acceptable salt thereof.

More preferred are compounds of formula (Ia₂) wherein
Yₐ is absent;
   or a pharmaceutically acceptable salt thereof.

Even more preferred are compounds of formula (Ia₂) wherein
n is 3;
R₆ₐ and R₇ₐ are lower alkyl;
m is zero or 1;
Xₐ is hydroxy, cyano or free or esterified carboxy;
   or a pharmaceutically acceptable salt thereof.

Most preferred are compounds of formula (Ia₂) wherein
R₆ₐ and R₇ₐ are methyl;
   or a pharmaceutically acceptable salt thereof.

Especially preferred are compounds of formula (Ia₂) wherein
R₁ is hydrogen or —C(O)R₂ in which R₂ is monocyclic aryl;
   or a pharmaceutically acceptable salt thereof.

Preferred are also compounds in the A group having the formula

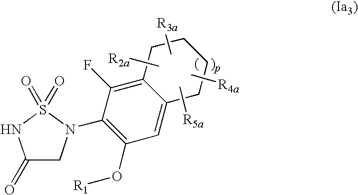

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the ring atoms to which they are attached form a 3- to 7-membered fused ring; or
   R₂ₐ and R₃ₐ combined are alkylene which together with the carbon atom to which they are attached form a 3- to 7-membered spirocyclic ring;
   p is zero or 1;
   or a pharmaceutically acceptable salt thereof.

Preferred are compounds of formula (Ia₃) wherein
R₄ₐ and R₅ₐ are hydrogen;
   or a pharmaceutically acceptable salt thereof.

Preferred are also compounds of formula (Ia₃) wherein

R₂ₐ and R₃ₐ are, independently from each other, hydrogen, halogen or (C₁₋₄)alkyl optionally substituted by at least one halogen;
or a pharmaceutically acceptable salt thereof.

Preferred are also compounds of formula (Ia₃) wherein
p is 1;
   or a pharmaceutically acceptable salt thereof.

Further preferred are compounds of formula (Ia₃) having the formula

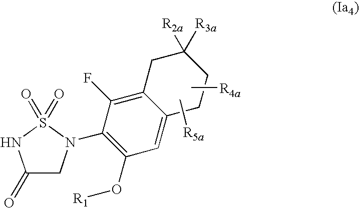

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ₐ, R₃ₐ, R₄ₐ and R₅ₐ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ₐ and R₃ₐ combined are alkylene which together with the carbon atom to which they are attached form a 3- to 7-membered spirocyclic ring;
   or a pharmaceutically acceptable salt thereof.

Preferred are compounds of formula (Ia₄) wherein
R₄ₐ and R₅ₐ are hydrogen;
   or a pharmaceutically acceptable salt thereof.

Preferred are also compounds of formula (Ia4), designated as the B group, wherein

R₂ₐ and R₃ₐ are, independently from each other, hydrogen, halogen or (C₁₋₄)alkyl optionally substituted by at least one halogen;
or a pharmaceutically acceptable salt thereof.

Preferred are compounds in the B group wherein
R₁ is hydrogen or —C(O)R₂ in which R₂ is monocyclic aryl;
   or a pharmaceutically acceptable salt thereof.

Preferred are also compounds of formula (Ia₄), designated as the C group, wherein

R₂ₐ and R₃ₐ combined are alkylene which together with the carbon atom to which they are attached form a 3- to 5-membered spirocyclic ring;
or a pharmaceutically acceptable salt thereof.

Preferred are compounds in the C group, wherein
R₁ is hydrogen or —C(O)R₂ in which R₂ is monocyclic aryl;
   or a pharmaceutically acceptable salt thereof.

Preferred are also compounds of formula (Ia₄), designated as the D group, wherein
R₂ₐ is —Yₐ—(CH₂)ₙ—CR₆ₐR₇ₐ—(CH₂)ₘ—Xₐ in which
   Yₐ is oxygen or S(O)_{q} in which q is zero or an integer of 1 or 2; or
   Yₐ is trans CH═CH; or
   Yₐ is absent;
   n is an integer from 1 to 6;
   R₆ₐ and R₇ₐ are, independently from each other, hydrogen or lower alkyl; or
      R₆ₐ and R₇ₐ combined are alkylene which together with the carbon atom to which they are attached form a 3- to 7-membered ring;
m is zero or an integer of 1 or 2;
   Xₐ is hydroxy, alkoxy, cycloalkyl, cycloalkoxy, acyl, acyloxy, carbamoyl, cyano, trifluoromethyl, free or esterified carboxy, monocyclic aryl or heterocyclyl;
   or a pharmaceutically acceptable salt thereof.

Preferred are compounds in the D group wherein
R₃ₐ is hydrogen;
   or a pharmaceutically acceptable salt thereof.

Further preferred are compounds in the D group wherein
n is an integer of 2 or 3;
R₆ₐ and R₇ₐ are, independently from each other, hydrogen or lower alkyl;
m is zero or 1;
Xₐ is hydroxy, carbamoyl, cyano, trifluoromethyl, free or esterified carboxy, monocyclic aryl or heterocyclyl;
   or a pharmaceutically acceptable salt thereof.

More preferred are compounds in the D group wherein
Yₐ is absent;
   or a pharmaceutically acceptable salt thereof.

Even more preferred are compounds in the D group wherein
n is 3;
R₆ₐ and R₇ₐ are lower alkyl;
m is zero or 1;
Xₐ is hydroxy, cyano or free or esterified carboxy;
   or a pharmaceutically acceptable salt thereof.

Most preferred are compounds in the D group wherein
R₆ₐ and R₇ₐ are methyl;
   or a pharmaceutically acceptable salt thereof.

Especially preferred are compounds in the D group wherein
R₁ is hydrogen or —C(O)R₂ in which R₂ is monocyclic aryl;
   or a pharmaceutically acceptable salt thereof.

Preferred are also the compounds of formula (I), wherein
U and V are hydrogen;
W is aryloxy, arylthio or methyl substituted with monocyclic aryl;
   or a pharmaceutically acceptable salt thereof.

Further preferred are also the compounds of formula (I) having the formula

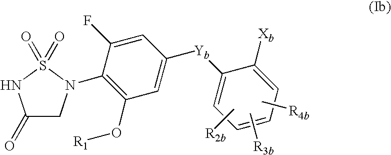

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R_{2b}, R_{3b} and R_{4b} are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R_{2b} and R_{3b} combined are alkylene which together with the ring atoms to which they are attached form a 5- to 7-membered fused ring provided R₂ and R₃ are attached to carbon atoms adjacent to each other; or
   R_{2b} and R_{3b} combined together with the carbon atom to which they are attached form a fused 5- to 6-membered aromatic or heteroaromatic ring provided R₂ and R₃ are attached to carbon atoms adjacent to each other;
   X_{b} is hydrogen, fluoro, cyano, or free or esterified carboxy; or
   X_{b} is —NR_{5b}C(O)R_{6b}, —NR_{5b}C(O)OR_{7b}, —NR_{5b}S(O)₂R_{8b}, —(CH₂)ᵣS(O)₂R_{9b}, —OS(O)₂R_{10b} or —OₛC(O)NR_{11b}R_{12b} in which
R_{5b} is hydrogen, lower alkyl, acyl, alkoxycarbonyl or sulfonyl;
   R_{6b}, R_{7b}, R_{8b}, R_{9b} and R_{10b} are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R_{6b}, R_{8b} and R_{9b} are, independently from each other, —NR_{13b}R_{14b} in which
   R_{13b} and R_{14b} are, independently from each other, hydrogen, alkyl, cycloalkyl, aralkyl, aryl or heterocyclyl; or
   R_{13b} and R_{14b} combined are alkylene which together with the nitrogen atom to which they are attached form a 4- to 7-membered ring,
   R_{11b} and R_{12b} are, independently from each other, hydrogen, alkyl, cycloalkyl, aralkyl, aryl or heterocyclyl; or
   R_{11b} and R_{12b} combined are alkylene which together with the nitrogen atom to which they are attached form a 4- to 7-membered ring;
   r and s are, independently from each other, zero or an integer of 1; or
   C—X_{b} is replaced by nitrogen;

### Y_{b} is O, S or CH₂;

or a pharmaceutically acceptable salt thereof.

Preferred are the compounds of formula (Ib) wherein
Y_{b} is CH₂;
   or a pharmaceutically acceptable salt thereof.

Further preferred are the compounds of formula (Ib) having the formula

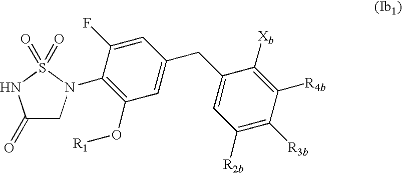

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R_{2b}, R_{3b} and R_{4b} are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R_{2b} and R_{3b} combined are alkylene which together with the ring atoms to which they are attached form a 5- to 7-membered fused ring; or
   R_{2b} and R_{3b} combined together with the carbon atom to which they are attached form a fused 5- to 6-membered aromatic or heteroaromatic ring;
   X_{b} is cyano; or
   X_{b} is —NR_{5b}C(O)R_{6b}, —NR_{5b}C(O)OR_{7b}, —NR_{5b}S(O)₂R_{8b}, —(CH₂)ᵣS(O)₂R_{9b} or —OS(O)₂R_{10b} in which
R_{5b} is hydrogen or lower alkyl;
   R_{6b}, R_{7b}, R_{8b}, R₁₃ and R_{10b} are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R_{6b}, R_{8b} and R_{9b} are, independently from each other, —NR_{13b}R_{14b} in which
   R_{13b} and R_{14b} are, independently from each other, hydrogen, alkyl, cycloalkyl, aralkyl, aryl or heterocyclyl; or
   R_{13b} and R_{14b} combined are alkylene which together with the nitrogen atom to which they are attached form a 4- to 7-membered ring;
   r is zero; or
   C—X_{b} is replaced by nitrogen;
   or a pharmaceutically acceptable salt thereof.

Preferred are the compounds of formula (Ib₁) wherein
X_{b} is cyano; or
   X_{b} is —NR_{5b}S(O)₂R_{8b} or —OS(O)₂R_{10b} in which
   R_{5b} is hydrogen or lower alkyl;
   R_{8b} and R_{10b} are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Especially preferred are the compounds of formula (Ib₁), designated as the E group, wherein
R_{5b} is hydrogen;
   or a pharmaceutically acceptable salt thereof.

Preferred are the compounds in the E group wherein
R_{8b} and R_{10b} are, independently from each other, monocyclic aryl or C₍₁₋₄₎alkyl;
   or a pharmaceutically acceptable salt thereof.

Further preferred are the compounds in the E group wherein
R₁ is hydrogen or —C(O)R₂ in which R₂ is monocyclic aryl;
   or a pharmaceutically acceptable salt thereof.

Especially preferred are also the compounds of formula (Ib₁), designated as the F group, wherein

R_{2b}, R_{3b} and R_{4b} are, independently from each other, hydrogen, halogen, hydroxy, monocyclic aryl, C₍₁₋₄₎alkoxy or C₍₁₋₄₎alkyl optionally substituted with at least one halogen;
or a pharmaceutically acceptable salt thereof.

Preferred are the compounds in the F group wherein
R_{5b} is hydrogen;
   or a pharmaceutically acceptable salt thereof.

Further preferred are the compounds in the F group wherein
R_{8b} and R_{10b} are, independently from each other, monocyclic aryl or C₍₁₋₄₎alkyl;
   or a pharmaceutically acceptable salt thereof.

More preferred are the compounds in the F group wherein
R₁ is hydrogen or —C(O)R₂ in which R₂ is monocyclic aryl;
   or a pharmaceutically acceptable salt thereof.

Preferred are also the compound of formula (I), designated as the G group, wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   U is alkoxy, alkylthio, alkylthiono, sulfonyl, cycloalkyl, aryl, aryloxy, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   W and V are, independently from each other, hydrogen, halogen, (C₁₋₃)alkyl or (C₁₋₃)alkoxy;
   or a pharmaceutically acceptable salt thereof.

Preferred are the compounds in the G group, designated as the H group, wherein
U is —Y_{c}—(CH₂)ₚ—CR_{2c}R_{3c}—(CH₂)ₜ—X_{c} in which
   Y_{c} is oxygen or S(O)ᵥ in which v is zero or an integer of 1 or 2; or
   Y_{c} is C≡C; or
   Y_{c} is absent;
   p and t are, independently from each other, zero or an integer from 1 to 8;
   R_{2c} and R_{3c} are, independently from each other, hydrogen or lower alkyl; or
      R_{2c} and R_{3c} combined are alkylene which together with the carbon atom to which they are attached form a 3- to 7-membered ring;
      X_{c} is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
      or a pharmaceutically acceptable salt thereof.

Preferred are the compounds in the H group wherein
R_{2c} and R_{3c} are hydrogen;
   or a pharmaceutically acceptable salt thereof.

Further preferred are the compounds in the H group wherein
p is zero or an integer from 1 to 3;
t is zero or 1;
R₂, and R₃, are, independently from each other, hydrogen or lower alkyl;
   X_{c} is hydroxy, carbamoyl, cyano, trifluoromethyl, free or esterified carboxy, heterocyclyl, monocyclic aryl or monocyclic aryloxy;
   or a pharmaceutically acceptable salt thereof.

Especially preferred are the compounds in the H group, designated as the I group, wherein
Y_{c} is C≡C; or
Y_{c} is absent;
   or a pharmaceutically acceptable salt thereof.

Preferred are the compounds in the I group wherein
Y_{c} is absent;
p is an integer of 5 or 6;
t is zero or 1;
R_{2c} and R_{3c} are lower alkyl;
X_{c} is hydroxy, cyano or free or esterified carboxy;
   or a pharmaceutically acceptable salt thereof.

Further preferred are the compounds in the I group wherein
R_{2c} and R_{3c} are methyl;
   or a pharmaceutically acceptable salt thereof.

Especially preferred are the compounds in the I group wherein
R₁ is hydrogen or —C(O)R₂ in which R₂ is monocyclic aryl;
   or a pharmaceutically acceptable salt thereof.

Preferred are also the compounds in the I group, designated as the J group, wherein
Y_{c} is absent;
p is an integer of 4 or 5;
t is zero;
R₂, and R₃, are hydrogen;
X_{c} is monocyclic aryloxy;
   or a pharmaceutically acceptable salt thereof.

Preferred are the compounds in the J group wherein
R₁ is hydrogen or —C(O)R₂ in which R₂ is monocyclic aryl;
   or a pharmaceutically acceptable salt thereof.

Preferred are also the compounds in the J group, designated as the K group, wherein
Y_{c} is C≡C;
p is an integer of 2 or 3;
t is zero;
R_{2c} and R_{3c} are hydrogen;
X_{c} is hydroxy, cyano or free or esterified carboxy;
   or a pharmaceutically acceptable salt thereof.

Preferred are the compounds in the K group wherein

R₁ is hydrogen or —C(O)R₂ in which R₂ is monocyclic aryl;

or a pharmaceutically acceptable salt thereof.

Preferred are also the compounds in the G group, designated as the L group, wherein Q_{c} is monocyclic aryl or 5- to 6-membered heterocyclic ring;

or a pharmaceutically acceptable salt thereof.

Preferred are the compounds in the L group, designated as the M group, wherein
R₂, and R₃, are hydrogen;
   or a pharmaceutically acceptable salt thereof.

Preferred are the compounds in the M group having the formula

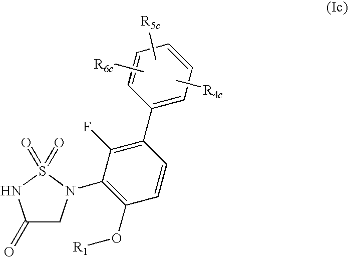

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R_{4c}, R_{5c}, and R_{6c} are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or C—R_{4c}, C—R_{5c} and C—R_{6c} are, independently from each other, replaced by nitrogen;
   or a pharmaceutically acceptable salt thereof.

Preferred are the compounds of formula (Ic) wherein
R_{4c} and R_{5c} are hydrogen;
   or a pharmaceutically acceptable salt thereof.

Preferred are also the compounds of formula (Ic) wherein
R₁ is hydrogen or —C(O)R₂ in which R₂ is monocyclic aryl;
   or a pharmaceutically acceptable salt thereof.

Preferred are also the compounds in the M group having the formula

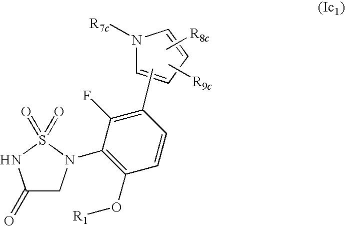

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R_{7c} is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   R_{8c} and R_{9c} are, independently from each other, hydrogen or lower alkyl; or C—R_{8c} and C—R_{9c} are, independently from each other, replaced by nitrogen;
   or a pharmaceutically acceptable salt thereof.

Preferred are the compounds of formula (Ic₁) wherein

C—R_{8c} is replaced by nitrogen;
R_{9c} is hydrogen;
or a pharmaceutically acceptable salt thereof.

Further preferred are the compounds of formula (Ic₁) having the formula

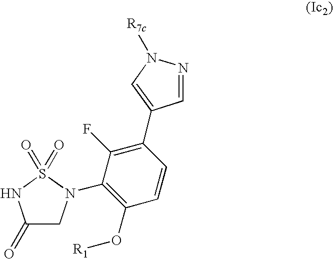

wherein
R₁ is hydrogen, —C(O)R₂, —C(O)NR₃R₄ or —C(O)OR₅ in which
   R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R_{7c} is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Preferred are the compounds of formula (Ic₂) wherein
R_{7c} is —(CH₂)ₚ—CR_{10c}R_{11c}—(CH₂)ₜ-Z_{c} in which
   p and t are, independently from each other, zero or an integer from 1 to 6;
   R_{10c} and R_{11c} are, independently from each other, hydrogen or lower alkyl; or
      R_{10c} and R_{11c} combined are alkylene which together with the carbon atom to which they are attached form a 3- to 7-membered ring;
      Z_{c} is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
      or a pharmaceutically acceptable salt thereof.

Further preferred are the compounds of formula (Ic₂) wherein
p is an integer from 1 to 3;
t is zero or 1;
R_{10c} and R_{11c} are, independently from each other, hydrogen or lower alkyl;
   Z_{c} is hydroxy, carbamoyl, cyano, trifluoromethyl, free or esterified carboxy, heterocyclyl, monocyclic aryl or monocyclic aryloxy;
   or a pharmaceutically acceptable salt thereof.

More preferred are the compounds of formula (Ic₂) wherein
R_{10c} and R_{11c} are hydrogen;
Z_{c} is hydroxy, cyano or free or esterified carboxy;
   or a pharmaceutically acceptable salt thereof.

Most preferred are the compounds of formula (Ic₂) wherein
R₁ is hydrogen or —C(O)R₂ in which R₂ is monocyclic aryl;
   or a pharmaceutically acceptable salt thereof.

Particular embodiments of the compounds are:
Methanesulfonic acid 2-[3-fluoro-5-hydroxy-4-(1,1,4-trioxo-1,2,5-thiadiazolidin-2-yl)-benzyl]-4-methylphenyl ester;
Methanesulfonic acid 2-[3-fluoro-5-hydroxy-4-(1,1,4-trioxo-1,2,5-thiadiazolidin-2-yl)-benzyl]-5-methylphenyl ester;
Methanesulfonic acid 2-[3-fluoro-5-hydroxy-4-(1,1,4-trioxo-1,2,5-thiadiazolidin-2-yl)-benzyl]-6-methylphenyl ester;
Methanesulfonic acid 2-[3-fluoro-5-hydroxy-4-(1,1,4-trioxo-1,2,5-thiadiazolidin-2-yl)-benzyl]-phenyl ester;
N-{2-[3-Fluoro-5-hydroxy-4-(1,1,4-trioxo-1,2,5-thiadiazolidin-2-yl)-benzyl]-5-methylphenyl}-methanesulfonamide;
N-{2-[3-Fluoro-5-hydroxy-4-(1,1,4-trioxo-1,2,5-thiadiazolidin-2-yl)-benzyl]-4-methylphenyl}-methanesulfonamide;
N-{2-[3-Fluoro-5-hydroxy-4-(1,1,4-trioxo-1,2,5-thiadiazolidin-2-yl)-benzyl]-phenyl}-methanesulfonamide;
5-(4-Benzyl-2-fluoro-6-hydroxy-phenyl)-1,1-dioxo-1,2,5-thiadiazolidin-3-one;
5-(2-Fluoro-6-hydroxy-4-methylphenyl)-1,1-dioxo-1,2,5-thiadiazolidin-3-one;
Benzoic acid 5-benzyl-3-fluoro-2-(1,1,4-trioxo-1,2,5-thiadiazolidin-2-yl)-phenyl ester;
Benzoic acid 3-fluoro-5-methyl-2-(1,1,4-trioxo-1,2,5-thiadiazolidin-2-yl)-phenyl ester;
5-(4-Cyclobutylmethyl-2-fluoro-6-hydroxyphenyl)-1,1-dioxo-1,2,5-thiadiazolidin-3-one potassium salt;
5-(4-Cyclohexylmethyl-2-fluoro-6-hydroxyphenyl)-1,1-dioxo-1,2,5-thiadiazolidin-3-one;
7-[2-Fluoro-4-hydroxy-3-(1,1,4-trioxo-1,2,5-thiadiazolidin-2-yl)-phenyl]-2,2-dimethylheptanenitrile;
5-(2,4-Difluoro-6-hydroxyphenyl)-1,1-dioxo-1,2,5-thiadiazolidin-3-one;
5-(1-Fluoro-3-hydroxy-7-methylnaphthalen-2-yl)-1,1-dioxo-1,2,5-thiadiazolidin-3-one;
5-(1-Fluoro-3-hydroxynaphthalen-2-yl)-1,1-dioxo-1,2,5-thiadiazolidin-3-one;
5-(7-Ethyl-1-fluoro-3-hydroxy-5,6,7,8-tetrahydronaphthalen-2-yl)-1,1-dioxo-1,2,5-thiadiazolidin-3-one;
5-[1-Fluoro-3-hydroxy-7-(5-hydroxy-4,4-dimethylpentyl)-naphthalen-2-yl]-1,1-dioxo-1,2,5-thiadiazolidin-3-one;
5-[8-Fluoro-6-hydroxy-7-(1,1,4-trioxo-1,2,5-thiadiazolidin-2-yl)-naphthalen-2-yl]-2,2-dimethyl-pentanoic acid;
Benzoic acid 4-fluoro-6-methyl-3-(1,1,4-trioxo-1,2,5-thiadiazolidin-2-yl)-naphthalen-2-yl ester;
Benzoic acid 6-ethyl-4-fluoro-3-(1,1,4-trioxo-1,2,5-thiadiazolidin-2-yl)-5,6,7,8-tetrahydronaphthalen-2-yl ester;
Benzoic acid 4-fluoro-3-(1,1,4-trioxo-1,2,5-thiadiazolidin-2-yl)-naphthalen-2-yl ester;
Benzoic acid 4-fluoro-6-(5-hydroxy-4,4-dimethylpentyl)-3-(1,1,4-trioxo-1,2,5-thiadiazolidin-2-yl)-naphthalen-2-yl ester;
Benzoic acid 3-fluoro-5-(2-methanesulfonyloxy-5-methylbenzyl)-2-(1,1,4-trioxo-1,2,5-thiadiazolidin-2-yl)-phenyl ester;
Benzoic acid 3-fluoro-5-(2-methanesulfonyloxy-4-methylbenzyl)-2-(1,1,4-trioxo-1,2,5-thiadiazolidin-2-yl)-phenyl ester;
Benzoic acid 4-(6-cyano-6,6-dimethylhexyl)-3-fluoro-2-(1,1,4-trioxo-1,2,5-thiadiazolidin-2-yl)-phenyl ester;
Benzoic acid 3-fluoro-5-(2-methanesulfonylamino-5-methylbenzyl)-2-(1,1,4-trioxo-1,2,5-thiadiazolidin-2-yl)-phenyl ester;
Benzoic acid 3-fluoro-5-(2-methanesulfonylamino-4-methylbenzyl)-2-(1,1,4-trioxo-1,2,5-thiadiazolidin-2-yl)-phenyl ester; and
Benzoic acid 3-fluoro-5-(2-methanesulfonyloxy-3-methylbenzyl)-2-(1,1,4-trioxo-1,2,5-thiadiazolidin-2-yl)-phenyl ester;
   or a pharmaceutically acceptable salt thereof.

### Category 3 PTP Inhibitors

The methods of the present invention can be practiced with the compounds of the formula

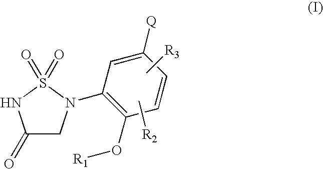

wherein

Q is alkoxy, alkylthio, alkylthiono, sulfonyl, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ and R₃ are, independently from each other, hydrogen, halogen, (C₁₋₃)alkyl or (C₁₋₃)alkoxy;
   or a pharmaceutically acceptable salt thereof.

Preferred are the compounds of formula (I), designated as the A group, wherein
Q is —Y—(CH₂)ₙ—CR₈R₉—(CH₂)ₘ—X in which
   Y is oxygen or S(O)_{q} in which q is zero or an integer of 1 or 2; or
   Y is C≡C; or
   Y is absent;
   n and m are, independently from each other, zero or an integer from 1 to 8;
   R₈ and R₉ are, independently from each other, hydrogen or lower alkyl; or
      R₈ and R₉ combined are alkylene which together with the carbon atom to which they are attached form a 3- to 7-membered ring;
      X is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
      or a pharmaceutically acceptable salt thereof.

Preferred are the compounds in the A group wherein
R₂ and R₃ are hydrogen;
   or a pharmaceutically acceptable salt thereof.

Further preferred are the compounds in the A group wherein
n is zero or an integer from 1 to 3;
m is zero or 1;
R₈ and R₉ are, independently from each other, hydrogen or lower alkyl;
   X is hydroxy, carbamoyl, cyano, trifluoromethyl, free or esterified carboxy, heterocyclyl, monocyclic aryl or monocyclic aryloxy;
   or a pharmaceutically acceptable salt thereof.

Especially preferred are the compounds in the A group, designated as the B group, wherein
Y is C≡C; or
Y is absent;
   or a pharmaceutically acceptable salt thereof.

Preferred are the compounds in the B group wherein
Y is absent;
n is an integer of 5 or 6;
m is zero or 1;
R₈ and R₉ are lower alkyl;
X is hydroxy, cyano or free or esterified carboxy;
   or a pharmaceutically acceptable salt thereof.

Further preferred are the compounds in the B group wherein
R₈ and R₉ are methyl;
   or a pharmaceutically acceptable salt thereof.

Especially preferred are the compounds in the B group wherein
R₁ is hydrogen or —C(O)R₄ in which R₄ is monocyclic aryl;
   or a pharmaceutically acceptable salt thereof.

Preferred are also the compounds in the B group, designated as the C group, wherein
Y is absent;
n is an integer of 4 or 5;
m is zero;
R₈ and R₉ are hydrogen;
X is monocyclic aryloxy;
   or a pharmaceutically acceptable salt thereof.

Preferred are the compounds in the C group wherein
R₁ is hydrogen or —C(O)R₄ in which R₄ is monocyclic aryl;
   or a pharmaceutically acceptable salt thereof.

Preferred are also the compounds in the B group, designated as the D group, wherein
Y is C≡C;
n is an integer of 2 or 3;
m is zero;
R₈ and R₉ are hydrogen;
X is hydroxy, cyano or free or esterified carboxy;
   or a pharmaceutically acceptable salt thereof.

Preferred are the compounds in the D group wherein
R₁ is hydrogen or —C(O)R₄ in which R₄ is monocyclic aryl;
   or a pharmaceutically acceptable salt thereof.

Preferred are the compounds of formula (I), designated as the E group, wherein

Q is monocyclic aryl or 5- to 6-membered heterocyclic ring;
or a pharmaceutically acceptable salt thereof.

Preferred are the compounds in the E group, designated as the G group, wherein
R₂ and R₃ are hydrogen;
   or a pharmaceutically acceptable salt thereof.

Preferred are the compounds in the G group having the formula

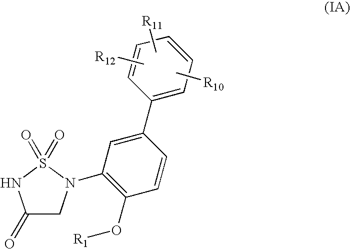

wherein
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₁₀, R₁₁ and R₁₂ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   C—R₁₀, C—R₁₁ and C—R₁₂ are, independently from each other, replaced by nitrogen;
   or a pharmaceutically acceptable salt thereof.

Preferred are the compounds of formula (IA) wherein
R₁₀ and R₁ are hydrogen;
   or a pharmaceutically acceptable salt thereof.

Preferred are also the compounds of formula (IA) wherein
R₁ is hydrogen or —C(O)R₄ in which R₄ is monocyclic aryl;
   or a pharmaceutically acceptable salt thereof.

Preferred are also the compounds in the G group having the formula

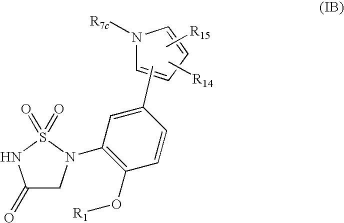

wherein
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   R₁₄ and R₁ₛ are, independently from each other, hydrogen or lower alkyl; or
   C—R₁₄ and C—R₁₅ are, independently from each other, replaced by nitrogen;
   or a pharmaceutically acceptable salt thereof.

Preferred are the compounds of formula (IB) wherein

C—R₁₄ is replaced by nitrogen;
R₁₅ is hydrogen;
or a pharmaceutically acceptable salt thereof.

Further preferred are the compounds of formula (IB) having the formula

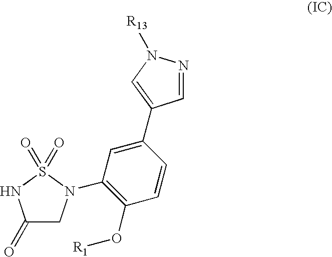

wherein
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₁₃ is hydrogen, sulfonyl, cycloalkyl, aryl, heterocyclyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Preferred are the compounds of formula (IC) wherein
R₁₃ is —(CH₂)ₙ—CR₁₆R₁₇—(CH₂)ₘ-Z in which
n and m are, independently from each other, zero or an integer from 1 to 6;
R₁₆ and R₁₇ are, independently from each other, hydrogen or lower alkyl; or
   R₁₆ and R₁₇ combined are alkylene which together with the carbon atom to which they are attached form a 3- to 7-membered ring;
   Z is hydroxy, alkoxy, cycloalkyl, cycloalkoxy, acyl, acyloxy, carbamoyl, optionally substituted amino, cyano, trifluoromethyl, free or esterified carboxy, heterocyclyl, monocyclic aryl or monocyclic aryloxy;
   or a pharmaceutically acceptable salt thereof.

Further preferred are the compounds of formula (IC) wherein
n is an integer from 1 to 3;
m is zero or 1;
R₁₆ and R₁₇ are, independently from each other, hydrogen or lower alkyl;
   Z is hydroxy, carbamoyl, cyano, trifluoromethyl, free or esterified carboxy, heterocyclyl, monocyclic aryl or monocyclic aryloxy;
   or a pharmaceutically acceptable salt thereof.

More preferred are the compounds of formula (IC) wherein
R₁₆ and R₁₇ are hydrogen;
Z is hydroxy, cyano or free or esterified carboxy;
   or a pharmaceutically acceptable salt thereof.

Most preferred are the compounds of formula (IC) wherein
R₁ is hydrogen or —C(O)R₄ in which R₄ is monocyclic aryl;
   or a pharmaceutically acceptable salt thereof.

Particular embodiments of the compounds are:
5-[2-Hydroxy-5-(1H-pyrrol-2-yl)-phenyl]-1,1-dioxo-1,2,5-thiadiazolidin-3-one;
5-(4-Hydroxybiphenyl-3-yl)-1,1-dioxo-1,2,5-thiadiazolidin-3-one;
5-[2-Hydroxy-5-(2H-pyrazol-3-yl)-phenyl]-1,1-dioxo-1,2,5-thiadiazolidin-3-one;
5-[2-Hydroxy-5-(1-methyl-1H-pyrazol-4-yl)-phenyl]-1,1-dioxo-1,2,5-thiadiazolidin-3-one;
5-(5-Furan-3-yl-2-hydroxyphenyl)-1,1-dioxo-1,2,5-thiadiazolidin-3-one;
5-[2-Hydroxy-5-(1H-pyrazol-4-yl)-phenyl]-1,1-dioxo-1,2,5-thiadiazolidin-3-one;
5-(4′-Acetyl-4-hydroxybiphenyl-3-yl)-1,1-dioxo-1,2,5-thiadiazolidin-3-one;
5-(4′-Benzoyl-4-hydroxybiphenyl-3-yl)-1,1-dioxo-1,2,5-thiadiazolidin-3-one;
5-[2-Hydroxy-5-(1H-pyrrol-3-yl)-phenyl]-1,1-dioxo-1,2,5-thiadiazolidin-3-one; Methanesulfonic acid 4′-hydroxy-3′-(1,1,4-trioxo-1,2,5-thiadiazolidin-2-yl)-biphenyl-3-yl ester;
5-(3′-Amino-4-hydroxybiphenyl-3-yl)-1,1-dioxo-1,2,5-thiadiazolidin-3-one;
5-(4-Hydroxy-2′-methylbiphenyl-3-yl)-1,1-dioxo-1,2,5-thiadiazolidin-3-one;
5-[2-Hydroxy-5-(1H-indol-2-yl)-phenyl]-1,1-dioxo-1,2,5-thiadiazolidin-3-one;
[4′-Hydroxy-3′-(1,1,4-trioxo-1,2,5-thiadiazolidin-2-yl)-biphenyl-3-yl]-acetonitrile;
4′-Hydroxy-3′-(1,1,4-trioxo-1,2,5-thiadiazolidin-2-yl)-biphenyl-3-carboxylic acid (2-cyanoethyl)-amide;
3-[4′-Hydroxy-3′-(1,1,4-trioxo-1,2,5-thiadiazolidin-2-yl)-biphenyl-3-yl]-propionic acid methyl ester;
4′-Hydroxy-3′-(1,1,4-trioxo-1,2,5-thiadiazolidin-2-yl)-biphenyl-3-carboxylic acid (2-carbamoylethyl)-amide;
5-[3′-(2-Aminoethyl)-4-hydroxybiphenyl-3-yl]-1,1-dioxo-1,2,5-thiadiazolidin-3-one;
5-(3′-Aminomethyl-4-hydroxybiphenyl-3-yl)-1,1-dioxo-1,2,5-thiadiazolidin-3-one;
5-(2-Hydroxy-5-pyridin-3-yl-phenyl)-1,1-dioxo-1,2,5-thiadiazolidin-3-one;
5-(4-Hydroxy-2′-methoxy-biphenyl-3-yl)-1,1-dioxo-1,2,5-thiadiazolidin-3-one;
5-(2-Hydroxy-5-pyridin-4-yl-phenyl)-1,1-dioxo-1,2,5-thiadiazolidin-3-one;
[4′-Hydroxy-3′-(1,4-trioxo-1,2,5-thiadiazolidin-2-yl)-biphenyl-4-yl]-acetic acid;
5-(4′-Chloro-4-hydroxybiphenyl-3-yl)-1,1-dioxo-1,2,5-thiadiazolidin-3-one;
5-(3′-Chloro-4-hydroxybiphenyl-3-yl)-1,1-dioxo-1,2,5-thiadiazolidin-3-one;
5-[2-Hydroxy-5-(6-methoxypyridin-3-yl)-phenyl]-1,1-dioxo-1,2,5-thiadiazolidin-3-one;
5-[5-(6-Fluoropyridin-3-yl)-2-hydroxyphenyl]-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionic acid ethyl ester;
5-(4-Hydroxy-3′-methylbiphenyl-3-yl)-1,1-dioxo-1,2,5-thiadiazolidin-3-one;
5-(3′-Fluoro-4-hydroxybiphenyl-3-yl)-1,1-dioxo-1,2,5-thiadiazolidin-3-one;
5-(4′-Fluoro-4-hydroxybiphenyl-3-yl-1,1-dioxo-1,2,5-thiadiazolidin-3-one;
5-(4-Hydroxy-4′-methylbiphenyl-3-yl)-1,1-dioxo-1,2,5-thiadiazolidin-3-one;
3-[4′-Hydroxy-3′-(1,1,4-trioxo-1,2,5-thiadiazolidin-2-yl)-biphenyl-3-yl]-propionitrile;
4′-Hydroxy-3′-(1,1,4-trioxo-1,2,5-thiadiazolidin-2-yl)-biphenyl-3-carbonitrile;
5-(4-Hydroxy-3′,5′-dimethylbiphenyl-3-yl)-1,1-dioxo-1,2,5-thiadiazolidin-3-one;
5-(4-Hydroxy-3′-methoxybiphenyl-3-yl)-1,1-dioxo-1,2,5-thiadiazolidin-3-one;
N-(2-Hydroxyethyl)-2-[4′-hydroxy-3′-(1,1,4-trioxo-1,2,5-thiadiazolidin-2-yl)-biphenyl-4-yl]-acetamide;
2,2,2-Trifluoro-N-[4′-hydroxy-3′-(1,1,4-trioxo-1,2,5-thiadiazolidin-2-yl)-biphenyl-3-yl]-acetamide;
1-Ethyl-3-[4′-hydroxy-3′-(1,1,4-trioxo-1,2,5-thiadiazolidin-2-yl)-biphenyl-3-yl]-urea;
1-Ethyl-3-[4′-hydroxy-3′-(1,1,4-trioxo-1,2,5-thiadiazolidin-2-yl)-biphenyl-3-ylmethyl]-urea;
[4′-Hydroxy-3′-(1,1,4-trioxo-1,2,5-thiadiazolidin-2-yl)-biphenyl-3-ylmethyl]-carbamic acid methyl ester;
N-[4′-Hydroxy-3′-(1,1,4-trioxo-1,2,5-thiadiazolidin-2-yl)-biphenyl-3-ylmethyl]-acetamide;
[4′-Hydroxy-3′-(1,1,4-trioxo-1,2,5-thiadiazolidin-2-yl)-biphenyl-3-ylmethyl]-carbamic acid benzyl ester;
1-Ethyl-3-[4′-hydroxy-3′-(1,1,4-trioxo-1,2,5-thiadiazolidin-2-yl)-biphenyl-4-yl]-urea;
3-[4′-Hydroxy-3′-(1,1,4-trioxo-1,2,5-thiadiazolidin-2-yl)-biphenyl-3-yl]-propionic acid;
5-{4-[4-Hydroxy-3-(1,1,4-trioxo-1,2,5-thiadiazolidin-2-yl)-phenyl]-pyrazol-1-yl}-pentanoic acid;
5-[2-Hydroxy-5-(1-propyl-1H-pyrazol-4-yl)-phenyl]-1,1-dioxo-1,2,5-thiadiazolidin-3-one;
5-[2-Hydroxy-5-(1-isobutyl-1H-pyrazol-4-yl)-phenyl]-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanoic acid ethyl ester;
5-{2-Hydroxy-5-[1-(4,4,4-trifluorobutyl)-1H-pyrazol-4-yl]-phenyl}-1,1-dioxo-1,2,5-thiadiazolidin-3-one;
5-{2-Hydroxy-5-[1-(3-methylbutyl)-1H-pyrazol-4-yl]-phenyl}-1,1-dioxo-1,2,5-thiadiazolidin-3-one;
5-{4-[4-Hydroxy-3-(1,1,4-trioxo-1,2,5-thiadiazolidin-2-yl)-phenyl]-1H-pyrazol-1-yl}-pentanenitrile;
4-{4-[4-Hydroxy-3-(1,14-trioxo-1,2,5-thiadiazolidin-2-yl)-phenyl]-1H-pyrazol-1-yl}-butyronitrile;
5-(2-Hydroxy-5-phenoxyphenyl)-1,1-dioxo-1,2,5-thiadiazolidin-3-one;
5-(2-Hydroxy-5-methoxyphenyl)-1,1-dioxo-1,2,5-thiadiazolidin-3-one;
5-(5-Benzyl-2-hydroxyphenyl)-1,1-dioxo-1,2,5-thiadiazolidin-3-one;
5-(2-Hydroxy-5-methylphenyl)-1,1-dioxo-1,2,5-thiadiazolidin-3-one;
5-(5-Hexyl-2-hydroxyphenyl)-1,1-dioxo-1,2,5-thiadiazolidin-3-one;
5-(5-Butyl-2-hydroxyphenyl)-1,1-dioxo-1,2,5-thiadiazolidin-3-one;
5-[2-Hydroxy-5-(tetrahydrofuran-3-yl)-phenyl]-1,1-dioxo-1,2,5-thiadiazolidin-3-one;
5-[5-(4-Fluorophenylethynyl)-2-hydroxyphenyl]-1,1-dioxo-1,2,5-thiadiazolidin-3-one;
6-[4-Hydroxy-3-(1,1,4-trioxo-1,2,5-thiadiazolidin-2-yl)-phenyl]-hex-5-ynenitrile;
6-[4-Hydroxy-3-(1,1,4-trioxo-1,2,5-thiadiazolidin-2-yl)-phenyl]-hex-5-ynoic acid;
5-[5-(3,3-Dimethyl-but-1-ynyl)-2-hydroxyphenyl]-1,1-dioxo-1,2,5-thiadiazolidin-3-one;
5-[2-Hydroxy-5-(5-methylhexyl)-phenyl]-1,1-dioxo-1,2,5-thiadiazolidin-3-one;
6-[4-Hydroxy-3-(1,1,4-trioxo-1,2,5-thiadiazolidin-2-yl)-phenyl]-hexanoic acid;
5-[5-(Benzylaminomethyl)-2-hydroxyphenyl]-1,1-dioxo-1,2,5-thiadiazolidin-3-one;
5-(5-Butylaminomethyl-2-hydroxyphenyl)-1,1-dioxo-1,2,5-thiadiazolidin-3-one;
5-{2-Hydroxy-5-[(2-methoxybenzylamino)-methyl]-phenyl}-1,1-dioxo-1,2,5-thiadiazolidin-3-one;
5-{5-[(2-Ethoxybenzylamino)-methyl]-2-hydroxyphenyl}-1,1-dioxo-1,2,5-thiadiazolidin-3-one;
5-{2-Hydroxy-5-[(2-isopropoxybenzylamino)-methyl]-phenyl}-1,1-dioxo-1,2,5-thiadiazolidin-3-one;
5-(2-Hydroxy-5-{[2-(1-methyl-2-phenylethoxy)-benzylamino]-methyl}-phenyl)-1,1-dioxo-1,2,5-thiadiazolidin-3-one;
5-[2-Hydroxy-5-(3-methylbutoxy)-phenyl]-1,1-dioxo-1,2,5-thiadiazolidin-3-one;
5-[2-Hydroxy-5-(4-methylpentyloxy)-phenyl]-1,1-dioxo-1,2,5-thiadiazolidin-3-one;
5-(2-Hydroxy-5-propoxyphenyl)-1,1-dioxo-1,2,5-thiadiazolidin-3-one;
2-Hydroxy-6-{4-[4-hydroxy-3-(1,1,4-trioxo-1,2,5-thiadiazolidin-2-yl)-phenyl]-butoxy}-N,N-dimethylbenzamide;
2-Hydroxy-6-{5-[4-hydroxy-3-(1,1,4-trioxo-1,2,5-thiadiazolidin-2-yl)-phenyl]-pentyloxy}-N,N-dimethylbenzamide;
2-Hydroxy-6-{6-[4-hydroxy-3-(1,1,4-trioxo-1,2,5-thiadiazolidin-2-yl)-phenyl]-hexyloxy}-N,N-dimethylbenzamide;
2-Fluoro-6-{6-[4-hydroxy-3-(1,1,4-trioxo-1,2,5-thiadiazolidin-2-yl)-phenyl]-hexyloxy}-N,N-dimethylbenzamide;
2-Hydroxy-6-{7-[4-hydroxy-3-(1,14-trioxo-1,2,5-thiadiazolidin-2-yl)-phenyl]-heptyloxy}-N,N-dimethylbenzamide;
5-(4-Hydroxy-4′-hydroxymethylbiphenyl-3-yl)-1,1-dioxo-1,2,5-thiadiazolidin-3-one;
5-(2-Hydroxy-4,5-dimethylphenyl)-1,1-dioxo-1,2,5-thiadiazolidin-3-one;
5-[4-Hydroxy-3-(1,1,4-trioxo-1,2,5-thiadiazolidin-2-yl)-phenyl]-2,2-dimethylpentanoic acid;
8-[4-Hydroxy-3-(1,1,4-trioxo-1,2,5-thiadiazolidin-2-yl)-phenyl]-2,2-dimethyloctanoic acid ethyl ester;
8-[4-Hydroxy-3-(1,1,4-trioxo-1,2,5-thiadiazolidin-2-yl)-phenyl]-2,2-dimethyloctanoic acid;
7-[4-Hydroxy-3-(1,1,4-trioxo-1,2,5-thiadiazolidin-2-yl)-phenyl]-2,2-dimethylheptanoic acid;
6-[4-Hydroxy-3-(1,1,4-trioxo-1,2,5-thiadiazolidin-2-yl)-phenyl]-2,2-dimethylhexanoic acid;
7-[4-Hydroxy-3-(1,1,4-trioxo-1,2,5-thiadiazolidin-2-yl)-phenyl]-2,2-dimethylheptanoic acid ethyl ester;
8-[4-Hydroxy-3-(1,14-trioxo-1,2,5-thiadiazolidin-2-yl)-phenyl]-2,2-dimethyloctanenitrile;
5-[2-Hydroxy-5-(6-hydroxy-6-methylheptyl)-phenyl]-1,1-dioxo-1,2,5-thiadiazolidin-3-one;
5-[2-Hydroxy-5-(7-hydroxy-6,6-dimethylheptyl)-phenyl]-1,1-dioxo-1,2,5-thiadiazolidin-3-one;
5-[2-Hydroxy-5-(5-hydroxy-5-methylhexyl)-phenyl]-1,1-dioxo-1,2,5-thiadiazolidin-3-one;
5-[2-hydroxy-5-(8-hydroxy-7,7-dimethyloctyl)-phenyl]-1,1-dioxo-1,2,5-thiadiazolidin-3-one;
7-[4-Hydroxy-3-(1,1,4-trioxo-1,2,5-thiadiazolidin-2-yl)-phenyl]-2,2-dimethylheptanenitrile;
5-[2-Hydroxy-5-(5-hydroxy-5-methylhex-1-ynyl)-phenyl]-1,1-dioxo-1,2,5-thiadiazolidin-3-one;
5-[2-Hydroxy-5-(2-pyridin-3-yl-ethyl)-phenyl]-1,1-dioxo-1,2,5-thiadiazolidin-3-one;
5-(2-Hydroxy-4-methyl-5-pentylphenyl)-1,1-dioxo-1,2,5-thiadiazolidin-3-one;
5-(2-Hydroxy-4-methyl-5-propylphenyl)-1,1-dioxo-1,2,5-thiadiazolidin-3-one;
5-(5-Heptyl-2-hydroxy-4-methylphenyl)-1,1-dioxo-1,2,5-thiadiazolidin-3-one;
5-[5-(2-Cyclohexylethyl)-2-hydroxy-4-methylphenyl]-1,1-dioxo-1,2,5-thiadiazolidin-3-one;
Benzoic acid 4-(7-hydroxy-6,6-dimethylheptyl)-2-(1,1,4-trioxo-1,2,5-thiadiazolidin-2-yl)-phenyl ester; and
Benzoic acid 4-(6-cyano-6,6-dimethylhexyl)-2-(1,1,4-trioxo-1,2,5-thiadiazolidin-2-yl)-phenylester;
   or a pharmaceutically acceptable salt thereof.

### Category 4 PTP Inhibitors

The methods of the invention can be practiced with the compounds of the formula

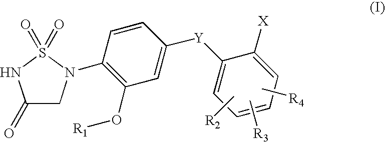

wherein
R₁ is hydrogen, —C(O)R₅, —C(O)NR₆R₇ or —C(O)OR₈ in which
   R₅ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₇ and R₈ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃ and R₄ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 5- to 7-membered fused ring provided R₂ and R₃ are attached to carbon atoms adjacent to each other; or
   R₂ and R₃ combined together with the carbon atom to which they are attached form a fused 5- to 6-membered aromatic or heteroaromatic ring provided R₂ and R₃ are attached to carbon atoms adjacent to each other;
   X is hydrogen, fluoro, cyano, or free or esterified carboxy; or
   X is —NR₉C(O)R₁₀, —NR₉C(O)OR₁₁, —NR₉S(O)₂R₂, —(CH₂)ₘS(O)₂R₁₃, —OS(O)₂R₁₄ or —OₙC(O)NR₁₅R₁₆ in which
R₉ is hydrogen, lower alkyl, acyl, alkoxycarbonyl or sulfonyl;
   R₁₀, R₁₁, R₁₂, R₁₃ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₁₀, R₁₂ and R₁₃ are, independently from each other, —NR₁₇R₁₈ in which
   R₁₇ and R₁₈ are, independently from each other, hydrogen, alkyl, cycloalkyl, aralkyl, aryl or heterocyclyl; or
   R₁₇ and R₁₈ combined are alkylene which together with the nitrogen atom to which they are attached form a 4- to 7-membered ring;
   R₁₅ and R₁₆ are, independently from each other, hydrogen, alkyl, cycloalkyl, aralkyl, aryl or heterocyclyl; or
   R₁₅ and R₁₆ combined are alkylene which together with the nitrogen atom to which they are attached form a 4- to 7-membered ring;
   m and n are, independently from each other, zero or an integer of 1; or
   C—X is replaced by nitrogen;

### Y is CH₂, O or S;

or a pharmaceutically acceptable salt thereof.

Preferred are the compounds of formula (I) wherein
Y is CH₂;
   or a pharmaceutically acceptable salt thereof.

Further preferred are the compounds of formula (I) having the formula

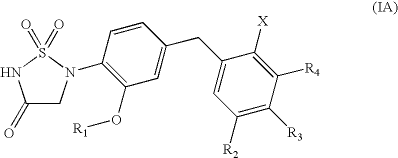

wherein
R₁ is hydrogen, —C(O)R₅, —C(O)NR₆R₇ or —C(O)OR₈ in which
   R₅ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₇ and R₈ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂, R₃ and R₄ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₂ and R₃ combined are alkylene which together with the ring atoms to which they are attached form a 5- to 7-membered fused ring; or
   R₂ and R₃ combined together with the carbon atom to which they are attached form a fused 5- to 6-membered aromatic or heteroaromatic ring;
   X is cyano; or
   X is —NR₉C(O)R₁₀, —NR₉C(O)OR₁₁, —NR₉S(O)₂R₁₂, —(CH₂)ₙS(O)₂R₁₃ or —OS(O)₂R₁₄ in which
R₉ is hydrogen or lower alkyl;
   R₁₀, R₁₁, R₁₂, R₁₃ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
   R₁₀, R₁₂ and R₁₃ are, independently from each other, —NR₁₇R₁₈ in which
   R₁₇ and R₁₈ are, independently from each other, hydrogen, alkyl, cycloalkyl, aralkyl, aryl or heterocyclyl; or
   R₁₇ and R₁₈ combined are alkylene which together with the nitrogen atom to which they are attached form a 4- to 7-membered ring;
   m is zero; or
   C—X is replaced by nitrogen;
   or a pharmaceutically acceptable salt thereof.

Preferred are the compounds of formula (IA) wherein
X is cyano; or
   X is —NR₉S(O)₂R₁₂ or —OS(O)₂R₁₄ in which
   R₉ is hydrogen or lower alkyl;
   R₁₂ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy;
   or a pharmaceutically acceptable salt thereof.

Especially preferred are the compounds of formula (IA), designated as the A group, wherein
R₉ is hydrogen;
   or a pharmaceutically acceptable salt thereof.

Preferred are the compounds in the A group wherein
R₁₂ and R₁₄ are, independently from each other, monocyclic aryl or C₍₁₋₄₎alkyl;
   or a pharmaceutically acceptable salt thereof.

Further preferred are the compounds in the A group wherein
R₁ is hydrogen or —C(O)R₅ in which R₅ is monocyclic aryl;
   or a pharmaceutically acceptable salt thereof.

Especially preferred are also the compounds of formula (IA), designated as the B group, wherein

R₂, R₃ and R₄ are, independently from each other, hydrogen, halogen, hydroxy, monocyclic aryl, C₍₁₋₄₎alkoxy or C₍₁₋₄₎alkyl optionally substituted with at least one halogen;
or a pharmaceutically acceptable salt thereof.

Preferred are the compounds in the B group wherein
R₉ is hydrogen;
   or a pharmaceutically acceptable salt thereof.

Further preferred are the compounds in the B group wherein
R₁₂ and R₁₄ are, independently from each other, monocyclic aryl or C₍₁₋₄₎alkyl;
   or a pharmaceutically acceptable salt thereof.

More preferred are the compounds in the B group wherein
R₁ is hydrogen or —C(O)R₅ in which R₅ is monocyclic aryl;
   or a pharmaceutically acceptable salt thereof.

Particular embodiments of the compounds are:
5-(4-Benzyl-2-hydroxyphenyl)-1,1-dioxo-1,2,5-thiadiazolidin-3-one;
5-[2-Hydroxy-4-(3-hydroxybenzyl)-phenyl]-1,1-dioxo-1,2,5-thiadiazolidin-3-one;
5-[2-Hydroxy-4-(3-methoxybenzyl)-phenyl]-1,1-dioxo-1,2,5-thiadiazolidin-3-one;
5-[4-(2-Fluoro-3-trifluoromethylbenzyl)-2-hydroxyphenyl]-1,1-dioxo-1,2,5-thiadiazolidin-3-one;
2-[3-Hydroxy-4-(1,1,4-trioxo-1,2,5-thiadiazolidin-2-yl)-benzyl]-benzonitrile;
5-[4-(2-Fluorobenzyl)-2-hydroxyphenyl]-1,1-dioxo-1,2,5-thiadiazolidin-3-one;
5-(2-Hydroxy-4-naphthalen-2-ylmethylphenyl)-1,1-dioxo-1,2,5-thiadiazolidin-3-one;
5-[2-Hydroxy-4-(3-trifluoromethylbenzylphenyl]-1,1-dioxo-1,2,5-thiadiazolidin-3-one;
5-[2-Hydroxy-4-(2-methylbenzyl)phenyl]-1,1-dioxo-1,2,5-thiadiazolidin-3-one;
5-[4-(4-Fluorobenzyl)-2-hydroxyphenyl]-1,1-dioxo-1,2,5-thiadiazolidin-3-one;
3-[3-Hydroxy-4-(1,1,4-trioxo-1,2,5-thiadiazolidin-2-yl)-benzyl]-benzoic acid methyl ester;
5-(4-Biphenyl-3-ylmethyl-2-hydroxyphenyl)-1,1-dioxo-1,2,5-thiadiazolidin-3-one;
5-[4-(3-Fluoro-4-methylbenzyl)-2-hydroxyphenyl]-1,1-dioxo-1,2,5-thiadiazolidin-3-one;
5-[2-Hydroxy-4-(4-methylbenzyl)phenyl]-1,1-dioxo-1,2,5-thiadiazolidin-3-one;
5-[2-Hydroxy-4-(4-hydroxybenzyl)phenyl]-1,1-dioxo-1,2,5-thiadiazolidin-3-one;
5-[4-(3-Fluorobenzyl)-2-hydroxyphenyl]-1,1-dioxo-1,2,5-thiadiazolidin-3-one;
5-[4-(4-tert-Butylbenzyl)-2-hydroxyphenyl]-1,1-dioxo-1,2,5-thiadiazolidin-3-one;
5-[4-{2-Benzenesulfonylmethylbenzyl)-2-hydroxyphenyl]-1,1-dioxo-1,2,5-thiadiazolidin-3-one;
5-[2-Hydroxy-4-(3-methylbenzyl)phenyl]-1,1-dioxo-1,2,5-thiadiazolidin-3-one;
{2-[3-Hydroxy-4-(1,1,4-trioxo-1,2,5-thiadiazolidin-2-yl)-benzyl]-phenyl}-carbamic acid tert-butyl ester;
N-{2-[3-Hydroxy-4-(1,1,4-trioxo-1,2,5-thiadiazolidin-2-yl)-benzyl]-phenyl}-C-phenyl-methanesulfonamide;
N-{2-[3-Hydroxy-4-(1,1,4-trioxo-1,2,5-thiadiazolidin-2-yl)-benzyl]-phenyl}-benzenesulfonamide;
Ethanesulfonic acid {2-[3-hydroxy-4-(1,1,4-trioxo-1,2,5-thiadiazolidin-2-yl)-benzyl]-phenyl}-amide;
Propane-1-sulfonic acid {2-[3-hydroxy-4-(1,1,4-trioxo-1,2,5-thiadiazolidin-2-yl)-benzyl]-phenyl}-amide;
Butane-1-sulfonic acid {2-[3-hydroxy-4-(1,1,4-trioxo-1,2,5-thiadiazolidin-2-yl)-benzyl]-phenyl}-amide;
C-Cyclohexyl-N-{2-[3-hydroxy-4-(1,1,4-trioxo-1,2,5-thiadiazolidin-2-yl)-benzyl]-phenyl}-methanesulfonamide;
N-{2-[3-Hydroxy-4-(1,1,4-trioxo-1,2,5-thiadiazolidin-2-yl)-benzyl]-phenyl}-methanesulfonamide;
N-{2-[3-Hydroxy-4-(1,1,4-trioxo-1,2,5-thiadiazolidin-2-yl)-benzyl]-phenyl}-4-isopropylbenzenesulfonamide;
N-{2-[3-Hydroxy-4-(1,1,4-trioxo-1,2,5-thiadiazolidin-2-yl)-benzyl]-phenyl}-aminosulfonamide;
N-{3-[3-Hydroxy-4-(1,1,4-trioxo-1,2,5-thiadiazolidin-2-yl)-benzyl]-naphthalen-2-yl}-methanesulfonamide;
N-{2-[3-Hydroxy-4-(1,1,4-trioxo-1,2,5-thiadiazolidin-2-yl)-benzyl]-phenyl}-acetamide;
4-tert-Butyl-N-{2-[3-hydroxy-4-(1,1,4-trioxo-1,2,5-thiadiazolidin-2-yl)-benzyl]-phenyl}-benzamide;
N-{2-[3-Hydroxy-4-(1,1,4-trioxo-1,2,5-thiadiazolidin-2-yl)-benzyl]-phenyl}-benzamide;
5-[4-(4-Ethylpyridin-2-ylmethyl)-2-hydroxyphenyl]-1,1-dioxo-1,2,5-thiadiazolidin-3-one;
5-[4-(6-Methoxypyridin-2-ylmethyl)-2-hydroxyphenyl]-1,1-dioxo-1,2,5-thiadiazolidin-3-one;
5-(2-Hydroxy-4-pyridin-2-ylmethylphenyl)-1,1-dioxo-1,2,5-thiadiazolidin-3-one;
5-[2-Hydroxy-4-(2-methanesulfonylbenzyl)-phenyl]-1,1-dioxo-1,2,5-thiadiazolidin-3-one;
N-{2-[3-Hydroxy-4-(1,1,4-trioxo-1,2,5-thiadiazolidin-2-yl)-benzyl]-phenyl}-N-methylmethanesulfonamide;
5-[2-Hydroxy-4-(2-methanesulfonylmethylbenzyl)-phenyl]-1,1-dioxo-1,2,5-thiadiazolidin-3-one;
5-{4-(3-Methansulfonylphenyl)methyl-2-hydroxyphenyl}-1,1-dioxo-1,2,5-thiadiazolidin-3-one;
C-{2-[3-Hydroxy-4-(1,1,4-trioxo-1,2,5-thiadiazolidin-2-yl)-benzyl]-phenyl}-N,N-dimethylmethanesulfonamide;
Methanesulfonic acid 2-[3-hydroxy-4-(1,1,4-trioxo-1,2,5-thiadiazoldin-2-yl)-benzyl]-phenyl ester;
Methanesulfonic acid 3-[3-hydroxy-4-(1,1,4-trioxo-1,2,5-thiadiazolidin-2-yl)-benzyl]-naphthalen-2-yl ester;
Methanesulfonic acid 2-[3-hydroxy-4-(1,1,4-trioxo-1,2,5-thiadiazolidin-2-yl)-benzyl]-naphthalen-1-yl ester;
Methanesulfonic acid 2-[3-hydroxy-4-(1,1,4-trioxo-1,2,5-thiadiazolidin-2-yl)-benzyl]-4-methylphenyl ester;
Methanesulfonic acid 1-[3-hydroxy-4-(1,1,4-trioxo-1,2,5-thiadiazolidin-2-yl)-benzyl]-naphthalen-2-yl ester;
Methanesulfonic acid 2-[3-hydroxy-4-(1,1,4-trioxo-1,2,5-thiadiazolidin-2-yl)-benzyl]-4-methoxyphenyl ester;
Methanesulfonic acid 2-[3-hydroxy-4-(1,1,4-trioxo-1,2,5-thiadiazolidin-2-yl)-benzyl]-6-methylphenyl ester;
Ethanesulfonic acid 2-[3-hydroxy-4-(1,1,4-trioxo-1,2,5-thiadiazolidin-2-yl)-benzyl]-4-methylphenyl ester;
Propane-1-sulfonic acid 2-[3-hydroxy-4-(1,1,4-trioxo-1,2,5-thiadiazolidin-2-yl)-benzyl]-4-methylphenyl ester;
Methanesulfonic acid 4-chloro-2-[3-hydroxy-4-(1,1,4-trioxo-1,2,5-thiadiazolidin-2-yl)-benzyl]-phenyl ester;
Methanesulfonic acid 2-[3-hydroxy-4-(1,1,4-trioxo-1,2,5-thiadiazolidin-2-yl)-benzyl]-5-methylphenyl ester;
Methanesulfonic acid 4-chloro-2-[3-hydroxy-4-(1,1,4-trioxo-1,2,5-thiadiazolidin-2-yl)-benzyl]-6-methylphenyl ester;
Ethanesulfonic acid 2-[3-hydroxy-4-(1,1,4-trioxo-1,2,5-thiadiazolidin-2-yl)-benzyl]-phenyl ester;
Propane-1-sulfonic acid 2-[3-hydroxy-4-(1,1,4-trioxo-1,2,5-thiadiazolidin-2-yl)-benzyl]-phenyl ester;
5-[4-(2-Fluoro-4-methylbenzyl)-2-hydroxyphenyl]-1,1-dioxo-1,2,5-thiadiazolidin-3-one;
3-[3-Hydroxy-4-(1,1,4-trioxo-1,2,5-thiadiazolidin-2-yl)-benzyl]-N-methylbenzamide potassium salt;
3-[3-Hydroxy-4-(1,1,4-trioxo-1,2,5-thiadiazolidin-2-yl)-benzyl]-benzoic acid dipotassium salt;
2-[3-Hydroxy-4-(1,1,4-trioxo-1,2,5-thiadiazolidin-2-yl)-benzyl]-benzoic acid;
5-[4-(2,5-Difluorobenzyl)-2-hydroxyphenyl]-1,1-dioxo-1,2,5-thiadiazolidin-3-one;
5-[4-(3-Ethylbenzyl)-2-hydroxyphenyl]-1,1-dioxo-1,2,5-thiadiazolidin-3-one;
5-(2-Hydroxy-4-phenoxyphenyl)-1,1-dioxo-1,2,5-thiadiazolidin-3-one potassium salt;
2-Hydroxy-6-[3-hydroxy-4-(1,1,4-trioxo-1,2,5-thiadiazolidin-2-yl)-benzyl]-benzonitrile;
2-[3-Hydroxy-4-(1,1,4-trioxo-1,2,5-thiadiazolidin-2-yl)-benzyl]-4-trifluoromethylbenzonitrile;
2-[3-Hydroxy-4-(1,1,4-trioxo-1,2,5-thiadiazolidin-2-yl)-benzyl]-4-methylbenzonitrile;
2-[3-Hydroxy-4-(1,1,4-trioxo-1,2,5-thiadiazolidin-2-yl)-benzyl]-6-methyl-benzonitrile;
2-[3-Hydroxy-4-(1,1,4-trioxo-1,2,5-thiadiazolidin-2-yl)-benzyl]-4-trifluoromethylbenzonitrile;
5-(2-Hydroxy-4-phenylsulfanylphenyl)-1,1-dioxo-1,2,5-thiadiazolidin-3-one;
2-[3-Hydroxy-4-(1,1,4-trioxo-1,2,5-thiadiazolidin-2-yl)-phenylsulfanyl]-4-trifluoromethylbenzonitrile;
2-[3-Hydroxy-4-(1,1,4-trioxo-1,2,5-thiadiazolidin-2-yl)-phenylsulfanyl]-6-trifluoromethylbenzonitrile;
Methanesulfonic acid 2-[3-diethylcarbamoyloxy-4-(1,1,4-trioxo-1,2,5-thiadiazolidin-2-yl)-benzyl]-phenyl ester;
Methanesulfonic acid 2-[3-isopropoxycarbonyloxy-4-(1,1,4-trioxo-1,2,5-thiadiazolidin-2-yl)-benzyl]-phenyl ester;
N-{4-Chloro-2-[3-hydroxy-4-(1,1,4-trioxo-1,2,5-thiadiazolidin-2-yl)-benzyl]-phenyl}-methanesulfonamide;
N-{2-[3-Hydroxy-4-(1,1,4-trioxo-1,2,5-thiadiazolidin-2-yl)-benzyl]-4-methylphenyl}-methanesulfonamide;
N-{4-Fluoro-2-[3-hydroxy-4-(1,1,4-trioxo-1,2,5-thiadiazolidin-2-yl)-benzyl]-phenyl}-methanesulfonamide;
N-{4-Fluoro-2-[3-hydroxy-4-(1,1,4-trioxo-1,2,5-thiadiazolidin-2-yl)-benzyl]-phenyl}-benzenesulfonamide;
Ethanesulfonic acid {4-fluoro-2-[3-hydroxy-4-(1,1,4-trioxo-1,2,5-thiadiazolidin-2-yl)-benzyl]-phenyl}-amide;
Propane-2-sulfonic acid {4-fluoro-2-[3-hydroxy-4-(1,1,4-trioxo-1,2,5-thiadiazolidin-2-yl)-benzyl]-phenyl}-amide;
Propane-1-sulfonic acid {2-[3-hydroxy-4-(1,1,4-trioxo-1,2,5-thiadiazolidin-2-yl)-benzyl]-4-methylphenyl}-amide;
N-{4-Fluoro-2-[3-hydroxy-4-(1,1,4-trioxo-1,2,5-thiadiazolidin-2-yl)-benzyl]-phenyl}-C-phenyl-methanesulfonamide;
Ethanesulfonic acid {4-chloro-2-[3-hydroxy-4-(1,1,4-trioxo-1,2,5-thiadiazolidin-2-yl)-benzyl]-phenyl}-amide;
Propane-2-sulfonic acid {4-chloro-2-[3-hydroxy-4-(1,1,4-trioxo-1,2,5-thiadiazolidin-2-yl)-benzyl]-phenyl}-amide;
Propane-1-sulfonic acid {4-chloro-2-[3-hydroxy-4-(1,1,4-trioxo-1,2,5-thiadiazolidin-2-yl)-benzyl]-phenyl}-amide;
Ethanesulfonic acid {4-chloro-2-[3-hydroxy-4-(1,1,4-trioxo-1,2,5-thiadiazolidin-2-yl)-benzyl]-6-methylphenyl}-amide;
N-{2-[3-Hydroxy-4-(1,1,4-trioxo-1,2,5-thiadiazolidin-2-yl)-benzyl]-6-methylphenyl}-methanesulfonamide;
N-{2-[3-Hydroxy-4-(1,1,4-trioxo-1,2,5-thiadiazolidin-2-yl)-benzyl]-4,6-dimethylphenyl}-methanesulfonamide;
Ethanesulfonic acid {2-[3-hydroxy-4-(1,1,4-trioxo-1,2,5-thiadiazolidin-2-yl)-benzyl]-6-methylphenyl}-amide;
Propane-1-sulfonic acid {2-[3-hydroxy-4-(1,1,4-trioxo-1,2,5-thiadiazolidin-2-yl)-benzyl]-6-methylphenyl}-amide;
Ethanesulfonic acid {2-[3-hydroxy-4-(1,1,4-trioxo-1,2,5-thiadiazolidin-2-yl)-benzyl]-4,6-dimethylphenyl}-amide;
N-{4-Chloro-2-[3-hydroxy-4-(1,1,4-trioxo-1,2,5-thiadiazolidin-2-yl)-benzyl]-6-methylphenyl}-methanesulfonamide;
N-{4-Chloro-2-[3-hydroxy-4-(1,1,4-trioxo-1,2,5-thiadiazolidin-2-yl)-benzyl]-6-methylphenyl}-methanesulfonamide;
N-{2-[3-Hydroxy-4-(1,1,4-trioxo-1,2,5-thiadiazolidin-2-yl)-benzyl]-5-methylphenyl}-methanesulfonamide;
N-{2-[3-Hydroxy-4-(1,1,4-trioxo-1,2,5-thiadiazolidin-2-yl)-benzyl]-6-methoxyphenyl}-methanesulfonamide;
N-{5-Chloro-2-[3-hydroxy-4-(1,1,4-trioxo-1,2,5-thiadiazolidin-2-yl)-benzyl]-phenyl}-methanesulfonamide;
Ethanesulfonic acid {2-[3-hydroxy-4-(1,1,4-trioxo-1,2,5-thiadiazolidin-2-yl)-benzyl]-4-methylphenyl}-amide;
Methanesulfonic acid 4-ethyl-2-[3-hydroxy-4-(1,1,4-trioxo-1,2,5-thiadiazolidin-2-yl)-benzyl]-phenyl ester;
Methanesulfonic acid 4-tert-butyl-2-[3-hydroxy-4-(1,1,4-trioxo-1,2,5-thiadiazolidin-2-yl)-benzyl]-phenyl ester;
Diethylcarbamic acid 2-[3-hydroxy-4-(1,1,4-trioxo-1,2,5-thiadiazolidin-2-yl)-benzyl]-4-methylphenyl ester;
Ethanesulfonic acid {4-ethyl-2-[3-hydroxy-4-(1,1,4-trioxo-1,2,5-thiadiazolidin-2-yl)-benzyl]-phenyl}-amide;
Propane-1-sulfonic acid {4-ethyl-2-[3-hydroxy-4-(1,1,4-trioxo-1,2,5-thiadiazolidin-2-yl)-benzyl]-phenyl}-amide;
N-{4-Ethyl-2-[3-hydroxy-4-(1,1,4-trioxo-1,2,5-thiadiazolidin-2-yl)-benzyl]-phenyl}-methanesulfonamide;
N-{4-Benzyl-2-[3-hydroxy-4-(1,1,4-trioxo-1,2,5-thiadiazolidin-2-yl)-benzyl]-phenyl}methanesulfonamide;
N-{3-[3-Hydroxy-4-(1,1,4-trioxo-1,2,5-thiadiazolidin-2-yl)-benzyl]-biphenyl-4-yl}-methanesulfonamide;
N-{2-[3-Hydroxy-4-(1,1,4-trioxo-1,2,5-thiadiazolidin-2-yl)-benzyl]-4-methoxyphenyl}-methanesulfonamide;
Ethanesulfonic acid {2-[3-hydroxy-4-(1,1,4-trioxo-1,2,5-thiadiazolidin-2-yl)-benzyl]-4-methoxyphenyl}-amide;
Propane-1-sulfonic acid {2-[3-hydroxy-4-(1,1,4-trioxo-1,2,5-thiadiazolidin-2-yl)-benzyl]-4-methoxyphenyl}-amide;
Methanesulfonic acid 5-[3-hydroxy-4-(1,1,4-trioxo-1,2,5-thiadiazolidin-2-yl)-benzyl]-7-methylindan-4-yl ester;
Methanesulfonic acid 6-[3-hydroxy-4-(1,1,4-trioxo-1,2,5-thiadiazolidin-2-yl)-benzyl]-indan-5-yl ester;
N-{2-[4-(1,1-dioxido-4-oxo-1,2,5-thiadiazolidin-2-yl)-3-hydroxybenzyl]-1,4-dimethylphenyl}sulfamide;
N-{2-[4-(1,1-dioxido-4-oxo-1,2,5-thiadiazolidin-2-yl)-3-hydroxybenzyl]-1-methyl-4-chlorophenyl}sulfamide;
N-{2-[4-(1,1-dioxido-4-oxo-1,2,5-thiadiazolidin-2-yl)-3-hydroxybenzyl]-4-ethylphenyl}sulfamide;
Methanesulfonic acid 2-[3-hydroxy-4-(1,1,4-trioxo-1,2,5-thiadiazolidin-2-yl)-benzyl]-6-isopropylphenyl ester;
Methanesulfonic acid 2-[3-hydroxy-4-(1,1,4-trioxo-1,2,5-thiadiazolidin-2-yl)-benzyl]-5-methylphenyl ester;
Methanesulfonic acid 2-chloro-6-[3-hydroxy-4-(1,1,4-trioxo-1,2,5-thiadiazolidin-2-yl)-benzyl]-phenyl ester;
Methanesulfonic acid 5-chloro-2-[3-hydroxy-4-(1,1,4-trioxo-1,2,5-thiadiazolidin-2-yl)-benzyl]-phenyl ester;
Methanesulfonic acid 2-[3-hydroxy-4-(1,1,4-trioxo-1,2,5-thiadiazolidin-2-yl)-benzyl]-5-methoxyphenyl ester;
Methanesulfonic acid 2-[3-hydroxy-4-(1,1,4-trioxo-1,2,5-thiadiazolidin-2-yl)-benzyl]-6-methoxyphenyl ester;
N-{2-Chloro-6-[3-hydroxy-4-(1,1,4-trioxo-1,2,5-thiadiazolidin-2-yl)-benzyl]-phenyl}-methanesulfonamide;
Methanesulfonic acid 2-[3-hydroxy-4-(1,1,4-trioxo-1,2,5-thiadiazolidin-2-yl)-benzyl]-4,6-dimethylphenyl ester;
Benzoic acid 5-benzyl-2-(1,1,4-trioxo-1,2,5-thiadiazolidin-2-yl)-phenyl ester;
Benzoic acid 5-(2-methanesulfonyloxybenzyl)-2-(1,1,4-trioxo-1,2,5-thiadiazolidin-2-yl)-phenyl ester;
Benzoic acid 5-(2-methanesulfonyloxy-5-methylbenzyl)-2-(1,1,4-trioxo-1,2,5-thiadiazolidin-2-yl)-phenyl ester;
Benzoic acid 5-(2-methanesulfonylamino-5-methylbenzyl)-2-(1,1,4-trioxo-1,2,5-thiadiazolidin-2-yl)-phenyl ester;
Benzoic acid 5-(2-methanesulfonylaminobenzyl)-2-(1,1,4-trioxo-1,2,5-thiadiazolidin-2-yl)-phenyl ester;
Benzoic acid 5-[2-(benzoylmethanesulfonylamino)-5-methylbenzyl]-2-(1,1,4-trioxo-1,2,5-thiadiazolidin-2-yl)-phenyl ester;
Benzoic acid 5-[2-(benzoylmethanesulfonylamino)-benzyl]-2-(1,1,4-trioxo-1,2,5-thiadiazolidin-2-yl)-phenyl ester;
2-Amino-3-methylbutyric acid 5-(2-methanesulfonyloxy-benzyl)-2-(1,1,4-trioxo-1,2,5-thiadiazolidin-2-yl)-phenyl ester;
Benzoic acid 5-(5-chloro-2-methanesulfonylamino-3-methylbenzyl)-2-(1,1,4-trioxo-1,2,5-thiadiazolidin-2-yl)-phenyl ester;
Benzoic acid 5-(2-methanesulfonylamino-3,5-dimethylbenzyl)-2-(1,1,4-trioxo-1,2,5-thiadiazolidin-2-yl)-phenyl ester;
2-Amino-3-methylbutyric acid 5-(2-methanesulfonyloxy-5-methylbenzyl-2-(1,1,4-trioxo-1,2,5-thiadiazolidin-2-yl)-phenyl ester;
Benzoic acid 5-(2-methanesulfonyloxy-3,5-dimethylbenzyl)-2-(1,1,4-trioxo-1,2,5-thiadiazolidin-2-yl)-phenyl ester;
Methanesulfonic acid 2-[3-methoxycarbonyloxy-4-(1,1,4-trioxo-1,2,5-thiadiazolidin-2-yl)-benzyl]-4-methylphenyl ester;
2-Amino-3-methylbutyric acid 5-(2-methanesulfonylamino-benzyl)-2-(1,1,4-trioxo-1,2,5-thiadiazolidin-2-yl)-phenyl ester;
2-(1,1-dioxido-4-oxo-1,2,5-thiadiazolidin-2-yl)-5-{2-[(methoxycarbonyl)(methylsulfonyl)-amino]-3,5-dimethylbenzyl}phenyl methyl carbonate;
Carbonic acid 5-(2-methanesulfonylamino-3,5-dimethylbenzyl)-2-(1,1,4-trioxo-1,2,5-thiadiazolidin-2-yl)-phenyl ester methyl ester;
Benzoic acid 5-(2-methanesulfonylamino-4-methylbenzyl)-2-(1,1,4-trioxo-1,2,5-thiadiazolidin-2-yl)-phenyl ester;
Benzoic acid 5-(2-methanesulfonyloxy-4-methylbenzyl)-2-(1,1,4-trioxo-1,2,5-thiadiazolidin-2-yl)-phenyl ester;
Benzoic acid 5-[2-(benzoylmethanesulfonylamino)-4-methylbenzyl]-2-(1,1,4-trioxo-1,2,5-thiadiazolidin-2-yl)-phenyl ester;
Benzoic acid 5-(2-methanesulfonyloxy-3-methylbenzyl)-2-(1,1,4-trioxo-1,2,5-thiadiazolidin-2-yl)-phenyl ester;
Benzoic acid 5-(5-chloro-2-methanesulfonyloxy-3-methylbenzyl)-2-(1,1,4-trioxo-1,2,5-thiadiazolidin-2-yl)-phenyl ester;
Benzoic acid 5-[2-(benzoylmethanesulfonylamino)-3-methylbenzyl]-2-(1,1,4-trioxo-1,2,5-thiadiazolidin-2-yl)-phenyl ester;
Benzoic acid 5-(2-methanesulfonylamino-3-methylbenzyl)-2-(1,1,4-trioxo-1,2,5-thiadiazolidin-2-yl)-phenyl ester;
2-Methylbenzoic acid 5-(2-methanesulfonyloxy-5-methylbenzyl)-2-(1,1,4-trioxo-1,2,5-thiadiazolidin-2-yl)-phenyl ester; and
5-(4-Benzyl-2-hydroxy-6-methylphenyl)-1,1-dioxo-1,2,5-thiadiazolidin-3-one;
   or a pharmaceutically acceptable salt thereof.

### Category 5 PTP Inhibitors

The methods of the invention can be practiced with the compounds of the formula

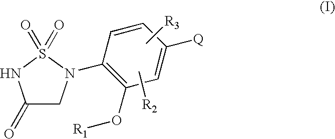

wherein

Q is:

### i) —X, or

ii)-Y—(CH₂)ₙ—(CR₉R₉)ₚ—(CH₂)ₘ-Z-X in which
Y is oxygen or S(O)_{q} in which q is zero or an integer of 1 or 2; or
Y is —C≡C— or —C═C—; or
Y is cyclopropyl or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
   R₈ and R₉ combined are alkylene which together with the carbon atom to which they are attached form a 3- to 7-membered ring;
   p is zero or an integer selected from 1 or 2
   Z is absent;

### Z is —C(O)—O—; or

### Z is —C(O)—; or

Z is —C(O)—NRα-alkylene- or —C(O)—NRα-alkylene-O—, wherein R [symbol] is H or lower alkyl; or
Z is —CO—NRα-(CH₂)_{n′}—(CR_{8′}R_{9′})_{p′}, —(CH₂)_{m′}—, or —C(O)—NRα-(CH₂)_{n′}—(CR_{8′}R_{9′})_{p′}, —(CH₂)ₘ′—O—, wherein p′ is zero or an integer of 1, n′ and m′ are, independently from each other, zero or an integer from 1 to 8, R_{8′} and R_{9′} are, independently from each other, hydrogen or lower alkyl, Rα is H or lower alkyl; or
Z is —NRα′-C(O), or —NRα′-C(O)—O—, wherein Rα′ is H or lower alkyl, or Rα′ and R₉ combined are alkylene which together with the carbon atom to which they are attached form a 3- to 7-membered ring; or

### Z is —C(O)—NH—NH—C(O)—O—; or

### Z is —S(O)₂—, or —S(O); or

Z is —NRβ—S(O)₂—, wherein Rβ is H, lower alkyl, or Rβ and R₉ combined are alkylene which together with the carbon atom to which they are attached form a 3- to 7-membered ring; or

### Z is —Nβ-S(O)₂—NH—C(O)—O—; or

Z is —NRγ-C(O)—NRγ′-; wherein Rγ′ is H, alkyl, aryl, heterocyclyl or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring or
Z is —NRτ-C(O)—NH—S(O)₂—, wherein Rτ is H or lower alkyl,
X is hydrogen, hydroxy, NH₂, halogen, alkoxy, alkylthio, alkyl, —S(O)—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
R₁ is hydrogen, —C(O)R₄, —C(O)NR₅R₆ or —C(O)OR₇ in which
   R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ and R₃ are, independently from each other, hydrogen, halogen, (C₁₋₃)alkyl or (C₁₋₃)alkoxy;
   or a pharmaceutically acceptable salt thereof,
   and wherein n+m+p is >1 or is 0, when X is aryl, and Y and Z are absent,
   n+m+p is not 0 when X is —O-aryl, and Y and Z are absent, or
   n+m+p is not 0 when X is —S-aryl, and Y and Z are absent, or
   n+m+p is not 0 when X is —CH₂-aryl, and Y and Z are absent, or
   n+m+p is not 0 when X is aryl, Z is absent and Y is —O— or Y is —S—, or
   wherein Q cannot be —CH₂-aryl, —S-aryl or —O-aryl.

Preferably, the orientation of the Z function is with the X group on the right side of the listed function -Z→X e.g. Z is —NRα′-C(O) means Z is —NRα′-C(O)—X.

Preferred are the compounds of formula (I), designated as the ALPHA group, wherein

Q is: —Y—(CH₂)ₙ—(CR₈R₉)ₚ—(CH₂)ₘ-Z-X in which
Y is oxygen or S(O)_{q} in which q is zero or an integer of 1 or 2; or
Y is —C≡C— or —C═C—; or
Y is cyclopropyl or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
   R₈ and R₉ combined are alkylene which together with the carbon atom to which they are attached form a 3- to 7-membered ring;
   p is zero or an integer selected from 1 or 2
   Z is absent;

### Z is —C(O)—O—; or

### Z is —C(O)—; or

Z is —C(O)—NRα-alkylene or —C(O)—NRα-alkylene-O—, wherein R [symbol] is H or lower alkyl; or
Z is —CO—NRα-(CH₂)_{n′}-(CR_{8′}R_{9′})_{p′}-(CH₂)_{m′}—, or —C(O)—NRα-(CH₂)_{n′}-(CR_{8′}R_{9′})_{p′}—(CH₂)_{m′}—O—, wherein p′ is zero or an integer of 1, n′ and m′ are, independently from each other, zero or an integer from 1 to 8, R_{8′} and R_{9′} are, independently from each other, hydrogen or lower alkyl, Rα is H or lower alkyl; or
Z is —NRα′-C(O)₂—, or —NRα′-C(O)—O—, wherein Rα′ is H or lower alkyl, or Rα′ and R₉ combined are alkylene which together with the carbon atom to which they are attached form a 3- to 7-membered ring; or

### Z is —C(O)—NH—NH—C(O)—O—; or

### Z is —S(O)₂—, or —S(O); or

Z is —NRβ-S(O)₂—, wherein Rβ is H, lower alkyl, or Rβ and R₉ combined are alkylene which together with the carbon atom to which they are attached form a 3- to 7-membered ring; or

### Z is —NH—S(O)₂—NH—C(O)—O—; or

Z is —NRγ—C(O)—NRγ′-; wherein Rγ′ is H, alkyl, aryl, heterocyclyl, or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring or
Z is —NRτ—C(O)—NH—S(O)₂—, wherein Rτ is H or lower alkyl,
X is hydrogen, hydroxy, NH₂, halogen, alkoxy, alkylthio, alkyl, —S(O)—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
R₁ is hydrogen, —C(O)R₄, —C(O)NR₅R₆ or —C(O)OR₇ in which R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
   R₂ and R₃ are, independently from each other, hydrogen, halogen, (C₁₋₃)alkyl or (C₁₋₃)alkoxy;
   or a pharmaceutically acceptable salt thereof,
   and wherein n+m+p is >1 or is 0, when X is aryl, and Y and Z are absent,
   n+m+p is not 0 when X is —O-aryl, and Y and Z are absent, or
   n+m+p is not 0 when X is —S-aryl, and Y and Z are absent, or
   n+m+p is not 0 when X is —CH₂-aryl, and Y and Z are absent, or
   n+m+p is not 0 when X is aryl, Z is absent and Y is —O— or Y is —S—, or
   wherein Q cannot be —CH₂-aryl, —S-aryl or —O-aryl.

Preferably, the orientation of the Z function is with the X group on the right side of the listed function -Z→X e.g. Z is —NRα′-C(O) means Z is —NRα′-C(O)—X.

Preferred are the compounds in the ALPHA group wherein;

Y is oxygen; or

### Y is —C≡C— or —C═C—; or

Y is cyclopropyl or
Y is absent; and
X is, hydrogen, hydroxy, NH₂, halogen, alkoxy, alkylthio, alkyl, —S(O)—OH, alkyl, cycloalkyl, cyano, trifluoromethyl, free or esterified carboxy, heterocyclyl, heteroaryl, heteroaralkyl, aryl, aralkyl, aryloxy;

Preferred are the compounds in the ALPHA group wherein
R₂ and R₃ are hydrogen;
   or a pharmaceutically acceptable salt thereof.

Preferred are the compounds in the ALPHA group wherein
R₁ is hydrogen;
   or a pharmaceutically acceptable salt thereof.

Further preferred are the compounds in the ALPHA group wherein
n is zero or an integer from 1 to 4;
m is zero or an integer from 1 to 4;
p is zero or 1;
   or a pharmaceutically acceptable salt thereof.

Especially preferred are the compounds in the ALPHA group, wherein
m+n+p is between 0 and 7 or preferably between 0 and 5,
   or a pharmaceutically acceptable salt thereof.

Preferred are the compounds of formula (I), designated as the A group, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or
Y is —C≡C— or —C═C—; or
Y is cyclopropyl; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl;
   p is zero or an integer selected from 1 or 2
   Z is absent;

### Z is —CO—O—; or

### Z is —CO—; or

X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the A group wherein

Y is oxygen; or
Y is cyclopropyl; or
Y is absent;
or a pharmaceutically acceptable salt thereof.

Preferred are the compounds in the A group wherein

R₈ and R₉ are, independently from each other, hydrogen, alkoxy, alkanoyl, alkoxycarbonyl, aralkyl, aryl, or alkyl;
or a pharmaceutically acceptable salt thereof.

Preferred are the compounds in the A group wherein

X is hydrogen, hydroxy, alkyl, heterocyclyl, heteroaryl, aryl;
or a pharmaceutically acceptable salt thereof.

Preferred are the compounds in the A group wherein
R₂ and R₃ are hydrogen;
   or a pharmaceutically acceptable salt thereof.

Preferred are the compounds in the A group wherein
R₁ is hydrogen;
   or a pharmaceutically acceptable salt thereof.

Further preferred are the compounds in the A group wherein
n is zero or an integer from 1 to 3;
m is zero or an integer from 1 to 3;
p is zero or 1;
   or a pharmaceutically acceptable salt thereof.

Especially preferred are the compounds in the A group, wherein
m+n+p is between 0 and 4,
   or a pharmaceutically acceptable salt thereof.

Other preferred compounds are the compounds in the A group, wherein
m+n+p is between 1 and 3, and
n is 1,
   or a pharmaceutically acceptable salt thereof.

Other preferred compounds are the compounds in the A group, wherein

X is phenyl.

Preferred are the compounds of formula (I), designated as B, wherein;

Q is —Y—(CH₂)ₙ—(CR₈R₉)ₚ—(CH₂)ₘZ-X in which
Y is oxygen or S(O)_{q} in which q is zero or an integer of 1 or 2; or
Y is —C≡C— or —C═C—; or
Y is cyclopropyl; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is absent;
X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
   or a pharmaceutically acceptable salt thereof.

Preferred are the compounds in the B group wherein
R₈ and R₉ are, independently from each other, hydrogen, alkoxy, alkanoyl, alkoxycarbonyl, aralkyl, or alkyl;
   or a pharmaceutically acceptable salt thereof.

Preferred are the compounds in the B group wherein

X is hydrogen, NH₂, hydroxy, alkylthio, —SO—OH, alkyl, cycloalkyl, cyano, trifluoromethyl, free or esterified carboxy, heterocyclyl, heteroaryl, aryl;
or a pharmaceutically acceptable salt thereof.

Preferred are the compounds in the B group wherein
R₂ and R₃ are hydrogen;
   or a pharmaceutically acceptable salt thereof.

Preferred are the compounds in the B group wherein
R₁ is hydrogen;
   or a pharmaceutically acceptable salt thereof.

Further preferred are the compounds in the B group wherein
n is zero or an integer from 1 to 3;
m is zero or an integer from 1 to 3;
p is zero or 1;
   or a pharmaceutically acceptable salt thereof.

Especially preferred are the compounds in the B group, wherein
m+n+p is between 0 and 6 or preferably 0 and 4,
   or a pharmaceutically acceptable salt thereof.

Other preferred compounds are the compounds in the B group, wherein
m+n is between 0 and 6 or preferably 0 and 4, and
p is 0,
   or a pharmaceutically acceptable salt thereof.

Other preferred compounds are the compounds in the B group, wherein

X is selected from phenyl or heteroaryl, preferably unsubstituted or substituted by at least one substituent e.g. one or two, which is preferably a substituent selected from carboxy, carbamoyl, and lower alkyl,
or a pharmaceutically acceptable salt thereof.

Other preferred compounds are the compounds in the B group, wherein
m+n is 1, 2 or 3, preferably 1 or 2,
m+m+p is preferably 2 or 3,
p is 1 or 0, and
X is cycloalkyl, heterocyclyl, heteroaryl, or aryl, preferably unsubstituted or substituted by at least one substituent e.g. one or two, which is preferably a substituent selected from sulfonamido, carboxy, carbamoyl, and lower alkyl,
   or a pharmaceutically acceptable salt thereof.

Other preferred compounds are the compounds in the B group, wherein
m+n is 1, 2 or 3, preferably 1 or 2,
m+n+p is 2, 3 or 4, preferably 2 or 3,
p is 1 or 0, and
X is aryl, preferably unsubstituted or substituted by at least one substituent e.g. one or two, which is preferably a substituent selected from sulfonamido, carboxy, carbamoyl, and lower alkyl,
   or a pharmaceutically acceptable salt thereof.

Other preferred compounds are the compounds in the B group, wherein
m+n is 1, 2 or 3, preferably 1 or 2,
p is 1 or 0, and
X is “amide” type heterocyclyl, cycloalkyl substituted by at least one substituent e.g. one or two, which is preferably sulfonamide, or aryl substituted by at least one substituent e.g. one or two, which is preferably sulfonamido
   or a pharmaceutically acceptable salt thereof.

Preferred are the compounds of formula (I), designated as the C, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or
Y is —C≡C— or —C═C—; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
   R₈ and R₉ combined are alkylene which together with the carbon atom to which they are attached form a 3- to 7-membered ring;
p is zero or an integer selected from 1 or 2
Z is —CO—NRα-alkylene- or —CO—NRα-alkylene-O, wherein R [symbol] is H or lower alkyl; or
Z is —CO—NRα-(CH₂)_{n′}—(CR_{8′}R_{9′})_{p′}—(CH₂)_{m′}—, or —CO—NRα-(CH₂)_{n′}—(CR_{8′}R_{9′})_{p′}—(CH₂)_{m′}—O—,
   wherein p′ is zero or an integer of 1, n′ and m′ are independently from each other, zero or an integer from 1 to 8, R_{8′} and R_{9′} are, independently from each other, hydrogen or lower alkyl, Rα is H or lower alkyl; or
   Z is —NRα′-CO—, or —NRα′-CO—O, wherein Rα′ is, H or lower alkyl, or Rα′ and R₉ combined are alkylene which together with the carbon atom to which they are attached form a 3- to 7-membered ring; or

### Z is —CO—NH—NH—CO—O—; or

X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the C group wherein

Y is absent;
or a pharmaceutically acceptable salt thereof.

Preferred are the compounds in the C group wherein

R₈ and R₉ are, independently from each other, hydrogen, alkanoylamino, aralkyl, aryl, or alkyl;
or a pharmaceutically acceptable salt thereof.

Preferred are the compounds in the C group wherein

X is hydrogen, alkyl, cycloalkyl, free or esterified carboxy, aryl, aralkyl, aryloxy;
or a pharmaceutically acceptable salt thereof.

Preferred are the compounds in the C group wherein
R₂ and R₃ are hydrogen;
   or a pharmaceutically acceptable salt thereof.

Preferred are the compounds in the C group wherein
R₁ is hydrogen;
   or a pharmaceutically acceptable salt thereof.

Further preferred are the compounds in the C group wherein
n is zero or an integer from 1 to 3;
m is zero or an integer from 1 to 3;
p is zero or 1;
   or a pharmaceutically acceptable salt thereof.

Especially preferred are the compounds in the C group, wherein
m+n+p is between 0 and 6 or preferably between 0 and 4,
   or a pharmaceutically acceptable salt thereof.

Other preferred compounds are the compounds in the C group, wherein

i) m+n+p is between 1 and 3 (i.e. 1, 2 or 3)
ii) m+n is between 1 and 3 (i.e. 1, 2 or 3) and p is 0
iii) m+n+p is between 1 and 3 (i.e. 1, 2 or 3) and p is 1
iv) m is 0, n is between 1 and 2 (i.e. 1, or 2) and p is 1 or a pharmaceutically acceptable salt thereof.

Especially preferred are the compounds in the C group, wherein

n′ and m′ are independently from each other, zero or an integer from 1 to 6, and
p′ is zero or an integer of 1, or a pharmaceutically acceptable salt thereof.

Especially preferred are the compounds in the C group, wherein

p′+n′+m′ is comprised between zero and 5, or between 3 and 5 i.e. 3, 4 or 5, or a pharmaceutically acceptable salt thereof.

Especially preferred are the compounds in the C group, wherein

n′ and m′ are independently from each other, zero or an integer from 1 to 6, preferably from 1 to 4,
or a pharmaceutically acceptable salt thereof.

Other preferred compounds are the compounds in the C group, wherein

n′+m′ is between 0 and 5, or between 3 and 5, preferably 4, and
p′ is 0,
or a pharmaceutically acceptable salt thereof.

Especially preferred are the compounds in the C group, wherein

X is phenyl, preferably unsubstituted or substituted preferably by at least one, e.g. one or two, of the substituents selected preferably from alkoxycarbonyl, carboxy, alkoxy, cyano, lower alkyl, (lower alkyl)-NHC(O)—, (lower alkyl)₂-NC(O)— and hydroxy.

Preferred are the compounds of formula (I), designated as the D group, wherein

Q is —Y—(CH₂)ₙ—(CR₈R₉)ₚ—(CH₂)ₘ-Z-X, in which
Y is absent;
n and m are, independently from each other, zero;
   p is zero;
   Z is absent;
   X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
   or a pharmaceutically acceptable salt thereof.

Preferred are the compounds in the D group wherein

X is halogen, cyano, trifluoromethyl, heterocyclyl, heteroaryl, aryl,
or a pharmaceutically acceptable salt thereof.

Preferred are the compounds in the D group wherein
R₂ and R₃ are hydrogen;
   or a pharmaceutically acceptable salt thereof.

Preferred are the compounds in the D group wherein
R₁ is hydrogen;
   or a pharmaceutically acceptable salt thereof.

Further preferred are the compounds in the D group wherein

X is aryl or heteroaryl, or a pharmaceutically acceptable salt thereof.

Especially preferred are the compounds in the D group, wherein

X is aryl substituted by an “amide” type heterocyclyl, or a pharmaceutically acceptable salt thereof.

Preferred are the compounds of formula (I), designated as the E group, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or
Y is —C≡C— or —C═C—; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
R₈ and R₉ combined are alkylene which together with the carbon atom to which they are attached form a 3- to 7-membered ring;
p is zero or an integer selected from 1 or 2
Z is —SO₂—, or —SO—; or
Z is —NRβ-SO₂—, wherein Rβ is H, lower alkyl, or Rβ and R₉ combined are alkylene which together with the carbon atom to which they are attached form a 3- to 7-membered ring preferably 5-, 6- or 7-membered ring; or

### Z is —NH₁—SO₂—NH—CO—O—; or

X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
or a pharmaceutically acceptable salt thereof.

Preferred are the compounds in the E group wherein

Y is absent; or a pharmaceutically acceptable salt thereof.

Preferred are the compounds in the E group wherein

R₈ and R₉ are, independently from each other, hydrogen, aralkyl, heteroaryl, heterocyclyl, heterocyclyl, carbamoyl; or
R₈ and R₉ combined are alkylene which together with the carbon atom to which they are attached form a 3- to 7-membered ring;
or a pharmaceutically acceptable salt thereof.

Preferred are the compounds in the E group wherein

X is hydrogen, alkyl, cycloalkyl, heteroaryl, aryl, aralkyl;
or a pharmaceutically acceptable salt thereof.

Preferred are the compounds in the E group wherein
R₂ and R₃ are hydrogen;
   or a pharmaceutically acceptable salt thereof.

Preferred are the compounds in the E group wherein
R₁ is hydrogen;
   or a pharmaceutically acceptable salt thereof.

Further preferred are the compounds in the E group wherein
n is zero or an integer from 1 to 4;
m is zero or an integer from 1 to 4;
p is zero or 1;
   or a pharmaceutically acceptable salt thereof.

Especially preferred are the compounds in the E group, wherein
m+n+p is between 0 and 7 or preferably between 0 and 5,
   or a pharmaceutically acceptable salt thereof.

Other preferred compounds are the compounds in the E group, wherein
i) m+n+p is 2 or 3, or
ii) m+n is 2 or 3, and p is 0, or
iii) n is 1 or 2, m is 0 or 1, and p is 1 when Rβ and R₉ combined are alkylene which together with the carbon atom to which they are attached form a 5-, 6- or 7-membered ring or a pharmaceutically acceptable salt thereof.
   Other preferred compounds are the compounds in the E group, wherein
   i) m+n is 1 or 2, m is 0 or 1, and p is 1, or
   ii) n is 1 or 2, m is 0 or 1, and p is 1 when R₈ is hydrogen and R₉ is selected from aralkyl, heteroaryl, heterocyclyl, heterocyclyl, or carbamoyl;
   or a pharmaceutically acceptable salt thereof.

Other preferred compounds are the compounds in the E group, wherein

X is selected from phenyl, biphenyl, benzyl, lower alkyl, methyl substituted by on or two pheny, ethyl substituted by on or two pheny, or methyl substituted by cycloalkyl

Preferred are the compounds of formula (I), designated as the F group, wherein

Q is —Y—(CH₂)ₙ—(CR₈R₉)ₚ—(CH₂)ₘ-Z-X, in which
Y is oxygen or S(O)_{q} in which q is zero or an integer of 1 or 2; or
Y is —C≡C— or —C═C—; or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, heterocyclyl, carbamoyl, aryl, or alkyl; or
   R₈ and R₉ combined are alkylene which together with the carbon atom to which they are attached form a 3- to 7-membered ring;
   p is zero or an integer selected from 1 or 2
   Z is —NRγ-CO—NRγ′-; wherein Rγ′ is H, alkyl, aryl, heterocyclyl, or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring; or
   Z is —NRτ-CO—NH—SO₂—, wherein Rτ is H or lower alkyl,
   X is hydrogen, hydroxy, NH₂, halogen, alkoxy, alkylthio, —SO—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
   or a pharmaceutically acceptable salt thereof.

Preferred are the compounds in the F group wherein

Y is absent; or a pharmaceutically acceptable salt thereof.

Preferred are the compounds in the F group wherein

R₈ and R₉ are, independently from each other, hydrogen;
or a pharmaceutically acceptable salt thereof.

Preferred are the compounds in the F group wherein

X is hydrogen, alkyl, cycloalkyl, heterocyclyl, aryl, aralkyl;
or a pharmaceutically acceptable salt thereof.

Preferred are the compounds in the F group wherein
R₂ and R₃ are hydrogen;
   or a pharmaceutically acceptable salt thereof.

Preferred are the compounds in the F group wherein

Rγ′ is H or lower alkyl, or a pharmaceutically acceptable salt thereof.

Preferred are the compounds in the F group wherein
R₁ is hydrogen;
   or a pharmaceutically acceptable salt thereof.

Especially preferred are the compounds in the F group, wherein
m+n+p is between 0 and 7 or preferably between 0 and 5 or between 2 and 3,
   or a pharmaceutically acceptable salt thereof.

Further preferred are the compounds in the F group wherein
n is zero or an integer from 1 to 4;
m is zero or an integer from 1 to 4;
p is zero or 1;
   or a pharmaceutically acceptable salt thereof.

Especially preferred are the compounds in the F group, wherein
m+n+p is 2 or 3, and
X is lower alkyl, phenyl, benzyl, or cyclohexyl,
   or a pharmaceutically acceptable salt thereof.
   Compound according to any of the above described groups wherein;
the term alkyl preferably refers to a lower alkyl,
aryl is preferably a phenyl, and/or
when R₈ and R₉ are present, at least one of R₈ or R₉ is hydrogen.

Particular embodiments of the compounds are: the below specific exemplified compounds,
3-{2-[3-Hydroxy-4-(1,1,4-trioxo-1,2,5-thiadiazolidin-2-yl)-phenyl]-ethyl}-benzamide
3-{2-[3-Hydroxy-4-(1,1,4-trioxo-1,2,5-thiadiazolidin-2-yl)-phenyl]-ethyl}-N-methyl benzamide
3-{2-[3-Hydroxy-4-(1,1,4-trioxo-1,2,5-thiadiazolidin-2-yl)-phenyl]-ethyl}-N,N-dimethylbenzamide
4-{2-[3-Hydroxy-4-(1,1,4-trioxo-1,2,5-thiadiazolidin-2-yl)-phenyl]-ethyl}-N,N-dimethylbenzamide
4-{2-[3-Hydroxy-4-(1,1,4-trioxo-1,2,5-thiadiazolidin-2-yl)-phenyl]-ethyl}-benzamide
4-{2-[3-Hydroxy-4-(1,1,4-trioxo-1,2,5-thiadiazolidin-2-yl)-phenyl]-ethyl}-N-methylbenzamide
3-{2-[3-Hydroxy-4-(1,1,4-trioxo-1,2,5-thiadiazolidin-2-yl)-phenyl]-ethyl}-benzoic acid
4-{2-[3-Hydroxy-4-(1,1,4-trioxo-1,2,5-thiadiazolidin-2-yl)-phenyl]-ethyl}-benzoic acid
4-{2-[3-Hydroxy-4-(1,1,4-trioxo-1,2,5-thiadiazolidin-2-yl)-phenyl]-ethyl}-benzonitrile
2-{2-[3-Hydroxy-4-(1,1,4-trioxo-1,2,5-thiadiazolidin-2-yl)-phenyl]-ethyl}-benzonitrile
5-(2-Hydroxy-4-phenethylphenyl)-1,1-dioxo-1,2,5-thiadiazolidin-3-one
5-{2-Hydroxy-4-[2-(3-methoxyphenyl)-ethyl]-phenyl}-1,1-dioxo-1,2,5-thiadiazolidin-3-one
5-{4-[2-(3-Fluorophenyl)-ethyl]-2-hydroxyphenyl}-1,1-dioxo-1,2,5-thiadiazolidin-3-one
5-{4-[2-(2-Fluorophenyl)-ethyl]-2-hydroxyphenyl}-1,1-dioxo-1,2,5-thiadiazolidin-3-one
5-[2-Hydroxy-4-(2-pentafluorophenylethyl)-phenyl]-1,1-dioxo-1,2,5-thiadiazolidin-3-one
5-[2-Hydroxy-4-(2-p-tolylethyl)-phenyl]-1,1-dioxo-1,2,5-thiadiazolidin-3-one
5-{2-Hydroxy-4-[2-(4-octylphenyl)-ethyl]-phenyl}-1,1-dioxo-1,2,5-thiadiazolidin-3-one
5-[4-(2-Biphenyl-4-yl-ethyl)-2-hydroxyphenyl]-1,1-dioxo-1,2,5-thiadiazolidin-3-one
5-{4-[2-(4-tert-Butylphenyl)-ethyl]-2-hydroxyphenyl}-1,1-dioxo-1,2,5-thiadiazolidin-3-one
5-{4-[2-(2,5-Dimethylphenyl)-ethyl]-2-hydroxyphenyl}-1,1-dioxo-1,2,5-thiadiazolidin-3-one
5-{4-[2-(2,4-Dimethylphenyl)-ethyl]-2-hydroxyphenyl}-1,1-dioxo-1,2,5-thiadiazolidin-3-one
5-{2-Hydroxy-4-[2-(4-trifluoromethylphenyl)-ethyl]-phenyl}-1,1-dioxo-1,2,5-thiadiazolidin-3-one
Acetic acid 4-{2-[3-hydroxy-4-(1,1,4-trioxo-1,2,5-thiadiazolidin-2-yl)-phenyl]-ethyl}-phenyl ester
5-{2-Hydroxy-4-[2-(4-phenoxyphenyl)-ethyl]-phenyl}-1,1-dioxo-1,2,5-thiadiazolidin-3-one
5-[2-Hydroxy-4-(2-pyridin-4-ylethyl)-phenyl]-1,1-dioxo-1,2,5-thiadiazolidin-3-one
5-[2-Hydroxy-4-(2-pyridin-3-yl-ethyl)-phenyl]-1,1-dioxo-1,2,5-thiadiazolidin-3-one
5-[2-Hydroxy-4-(2-naphthalenethyl)-phenyl]-1,1-dioxo-1,2,5-thiadiazolidin-3-one
5-[2-Hydroxy-4-(2-quinolin-3-yl-ethyl)-phenyl]-1,1-dioxo-1,2,5-thiadiazolidin-3-one
5-{4-[2-(4,6-Diamino-[1,3,5]triazin-2-yl)-ethyl]-2-hydroxy-phenyl}-1,1-dioxo-1,2,5-thiadiazolidin-3-one
5-[2-Hydroxy-4-(2-phenylpropyl)-phenyl]-1,1-dioxo-1,2,5-thiadiazolidin-3-one
5-{4-[2-(2-Aminophenyl)-propyl]-2-hydroxyphenyl}-1,1-dioxo-1,2,5-thiadiazolidin-3-one
3-[3-Hydroxy-4-(1,1,4-trioxo-1,2,5-thiadiazolidin-2-yl)-phenyl]-2-phenylpropionic acid ethyl ester
5-[2-Hydroxy-4-(1-methyl-2-phenylethyl)-phenyl]-1,1-dioxo-1,2,5-thiadiazolidin-3-one
5-{2-Hydroxy-4-[2-(6-methoxypyridin-2-yl)-ethyl]-phenyl}-1,1-dioxo-1,2,5-thiadiazolidin-3-one
5-[2-Hydroxy-4-((E)-2-pyridin-3-yl-vinyl)-phenyl]-1,1-dioxo-1,2,5-thiadiazolidin-3-one
5-[2-Hydroxy-4-(1-methoxy-2-phenylethyl)-phenyl]-1,1-dioxo-1,2,5-thiadiazolidin-3-one
5-[2-Hydroxy-4-(3-oxo-2-phenylbutyl)-phenyl]-1,1-dioxo-1,2,5-thiadiazolidin-3-one
5-{2-Hydroxy-4-[2-(2H-pyrazol-3-yl)-ethyl]-phenyl}-1,1-dioxo-1,2,5-thiadiazolidin-3-one
5-{2-Hydroxy-4-[2-(1H-pyrazol-4-yl)-ethyl]-phenyl}-1,1-dioxo-1,2,5-thiadiazolidin-3-one
5-{2-Hydroxy-4-[2-(1-methyl-1H-pyrazol-4-yl)-ethyl]-phenyl}-1,1-dioxo-1,2,5-thiadiazolidin-3-one
5-[2-Hydroxy-4-(2-thiazol-5-yl-ethyl)-phenyl]-1,1-dioxo-1,2,5-thiadiazolidin-3-one
5-{4-[2-(2,4-Dimethyl-thiazol-5-yl)-ethyl]-2-hydroxyphenyl}-1,1-dioxo-1,2,5-thiadiazolidin-3-one
5-[2-Hydroxy-4-(2-[1,2,4]triazol-yl-ethyl)-phenyl]-1,1-dioxo-1,2,5-thiadiazolidin-3-one
5-[2-Hydroxy-4-(2-imidazol-1-yl-ethyl)-phenyl]-1,1-dioxo-1,2,5-thiadiazolidin-3-one
5-{2-Hydroxy-4-[2-(2-methyl-thiazol-5-yl)-ethyl]-phenyl}-1,1-dioxo-1,2,5-thiadiazolidin-3-one
5-{2-Hydroxy-4-[2-(2-propyl-thiazol-5-yl)-ethyl]-phenyl}-1,1-dioxo-1,2,5-thiadiazolidin-3-one
5-(2-Hydroxy-4-{2-[4-methyl-2-(4-trifluoromethyl-phenyl)-thiazol-5-yl]-ethyl}-phenyl)-1,1-dioxo-1,2,5-thiadiazolidin-3-one
5-{2-Hydroxy-4-[2-(2-methyl-4-trifluoromethyl-thiazol-5-yl)-ethyl]-phenyl}-1,1-dioxo-1,2,5-thiadiazolidin-3-one
5-{4-[2-(1H-Benzoimidazol-2-yl)-ethyl]-2-hydroxyphenyl}-1,1-dioxo-1,2,5-thiadiazolidin-3-one
5-[2-Hydroxy-4-(3-phenylpropyl)-phenyl]-1,1-dioxo-1,2,5-thiadiazolidin-3-one
5-{4-[3-(3,4-Dimethoxyphenyl)-propyl]-2-hydroxyphenyl}-1,1-dioxo-1,2,5-thiadiazolidin-3-one
5-[2-Hydroxy-4-(2-methyl-3-phenylpropyl)-phenyl]-1,1-dioxo-1,2,5-thiadiazolidin-3-one
5-[2-Hydroxy-4-(3-hydroxy-3-phenylpropyl)-phenyl]-1,1-dioxo-1,2,5-thiadiazolidin-3-one
5-(2-Hydroxy-4-phenethyloxyphenyl)-1,1-dioxo-1,2,5-thiadiazolidin-3-one
5-[2-Hydroxy-4-(4-phenylbutyl)-phenyl]-1,1-dioxo-1,2,5-thiadiazolidin-3-one
{3-[3-Hydroxy-4-(1,1,4-trioxo-1,2,5-thiadiazolidin-2-yl)-phenyl]-propyl}-carbamic acid tert-butyl ester
5-[4-(3-Aminopropyl)-2-hydroxyphenyl]-1,1-dioxo-1,2,5-thiadiazolidin-3-one
{2-[3-Hydroxy-4-(1,1,4-trioxo-1,2,5-thiadiazolidin-2-yl)-phenyl]-ethyl}-carbamic acid tert-butyl ester
{(S)-1-Benzyl-2-[3-hydroxy-4-(1,1,4-trioxo-1,2,5-thiadiazolidin-2-yl)-phenyl]-ethyl}-carbamic acid tert-butyl ester
{3-[3-Hydroxy-4-(1,1,4-trioxo-1,2,5-thiadiazolidin-2-yl)-phenyl]-1,1-dimethylpropyl}-carbamic acid tert-butyl ester
2-{2-[3-Hydroxy-4-(1,1,4-trioxo-1,2,5-thiadiazolidin-2-yl)-phenyl]-ethyl}-piperidine-1-carboxylic acid tert-butyl ester
2-{2-[3-Hydroxy-4-(1,1,4-trioxo-1,2,5-thiadiazolidin-2-yl)-phenyl]-ethyl}-azepane-1-carboxylic acid tert-butyl ester
3-[3-Hydroxy-4-(1,1,4-trioxo-1,2,5-thiadiazolidin-2-yl)-benzyl]-piperidine-1-carboxylic acid tert-butyl ester
5-(2-Hydroxy-4-piperidin-3-ylmethylphenyl)-1,1-dioxo-1,2,5-thiadiazolidin-3-one
{(1R*,2S*)-2-[3-Hydroxy-4-(1,1,4-trioxo-1,2,5-thiadiazolidin-2-yl)-benzyl]-cyclohexyl}-carbamic acid tert-butyl ester
N-{2-[3-Hydroxy-4-(1,1,4-trioxo-1,2,5-thiadiazolidin-2-yl)-phenyl]-ethyl}-benzamide
4-Fluoro-N-{2-[3-hydroxy-4-(1,1,4-trioxo-1,2,5-thiadiazolidin-2-yl)-phenyl]-ethyl}-benzamide
N-{2-[3-Hydroxy-4-(1,1,4-trioxo-1,2,5-thiadiazolidin-2-yl)-phenyl]-ethyl}-acetamide
N-{2-[3-Hydroxy-4-(1,1,4-trioxo-1,2,5-thiadiazolidin-2-yl)-phenyl]-ethyl}-propionamide
N-{2-[3-Hydroxy-4-(1,1,4-trioxo-1,2,5-thiadiazolidin-2-yl)-phenyl]-ethyl}-isobutyramide
N-{2-[3-Hydroxy-4-(1,1,4-trioxo-1,2,5-thiadiazolidin-2-yl)-phenyl]-ethyl}-2,2-dimethyl-propionamide
Adamantane-1-carboxylic acid {2-[3-hydroxy-4-(1,1,4-trioxo-1,2,5-thiadiazolidin-2-yl)-phenyl]-ethyl}-amide
N-{3-[3-Hydroxy-4-(1,1,4-trioxo-1,2,5-thiadiazolidin-2-yl)-phenyl]-propyl}-acetamide
4-Fluoro-N-{3-[3-hydroxy-4-(1,1,4-trioxo-1,2,5-thiadiazolidin-2-yl)-phenyl]-propyl}-benzamide
-{3-[3-Hydroxy-4-(1,1,4-trioxo-1,2,5-thiadiazolidin-2-yl)-phenyl]-propyl}-propionamide
N-{3-[3-Hydroxy-4-(1,1,4-trioxo-1,2,5-thiadiazolidin-2-yl)-phenyl]-propyl}-isobutyramide
N-{3-[3-Hydroxy-4-(1,1,4-trioxo-1,2,5-thiadiazolidin-2-yl)-phenyl]-propyl}-2,2-dimethyl-propionamide
Adamantane-1-carboxylic acid {3-[3-hydroxy-4-(1,1,4-trioxo-1,2,5-thiadiazolidin-2-yl)-phenyl]-propyl}-amide
5-[2-Hydroxy-4-((S)-5-oxopyrrolidin-2-ylmethyl)-phenyl]-1,1-dioxo-1,2,5-thiadiazolidin-3-one
6-[3-Hydroxy-4-(1,1,4-trioxo-1,2,5-thiadiazolidin-2-yl)-benzyl]-1H-pyridin-2-one
6-[3-Hydroxy-4-(1,1,4-trioxo-1,2,5-thiadiazolidin-2-yl)-benzyl]-piperidin-2-one
7-[3-Hydroxy-4-(1,1,4-trioxo-1,2,5-thiadiazolidin-2-yl)-benzyl]-azepan-2-one
(R)-3-[3-Hydroxy-4-(1,1,4-trioxo-1,2,5-thiadiazolidin-2-yl)-benzyl]-3,4-dihydro-2H-isoquinolin-1-one
(S)-3-[3-Hydroxy-4-(1,1,4-trioxo-1,2,5-thiadiazolidin-2-yl)-benzyl]-2,3-dihydro-benzo[c]azepin-1-one
(R)-3-[3-Hydroxy-4-(1,1,4-trioxo-1,2,5-thiadiazolidin-2-yl)-benzyl]-2,3,4,5-tetrahydrobenzo[c]azepin-1-one
1-[3-Hydroxy-4-(1,1,4-trioxo-1,2,5-thiadiazolidin-2-yl)-benzyl]-1,2,4,5-tetrahydrobenzo[c]azepin-3-one
1-[3-Hydroxy-4-(1,1,4-trioxo-1,2,5-thiadiazolidin-2-yl)-benzyl]-1,3,4,5-tetrahydrobenzo[d]azepin-2-one
7-[3-Hydroxy-4-(1,1,4-trioxo-1,2,5-thiadiazolidin-2-yl)-benzyl]-6,7-dihydro-dibenzo[c,e]azepin-5-one
(S)-7-[3-Hydroxy-4-(1,1,4-trioxo-1,2,5-thiadiazolidin-2-yl)-benzyl]-6,7-dihydro-dibenzo[c,e]azepin-5-one
3-[3-Hydroxy-4-(1,1,4-trioxo-1,2,5-thiadiazolidin-2-yl)-benzyl]-3,4-dihydro-2H-naphtho[1,8-cd]azepin-1-one
5-{4-[2-(1-Acetylpiperidin-2-yl)-ethyl]-2-hydroxyphenyl}-1,1-dioxo-1,2,5-thiadiazolidin-3-one
N-{(1R*,2S*)-2-[3-Hydroxy-4-(1,1,4-trioxo-1,2,5-thiadiazolidin-2-yl)-benzyl]-cyclohexyl}-acetamide
N-{(S)-1-Benzyl-2-[3-hydroxy-4-(1,1,4-trioxo-1,2,5-thiadiazolidin-2-yl)-phenyl]-ethyl}-2,2,2-trifluoroacetamide
N-{4-[3-Hydroxy-4-(1,1,4-trioxo-1,2,5-thiadiazolidin-2-yl)-phenyl]-butyl}-phthalamic acid
2-{4-[3-Hydroxy-4-(1,1,4-trioxo-1,2,5-thiadiazolidin-2-yl)-phenyl]-butyl}-isoindole-1,3-dione
3-[3-Hydroxy-4-(1,1,4-trioxo-1,2,5-thiadiazolidin-2-yl)-phenyl]-N-isopropyl-N-methylpropionamide
5-{4-[3-(3,4-Dihydro-1H-isoquinolin-2-yl)-3-oxopropyl]-2-hydroxyphenyl}-1,1-dioxo-1,2,5-thiadiazolidin-3-one
N′-{3-[3-Hydroxy-4-(1,1,4-trioxo-1,2,5-thiadiazolidin-2-yl)-phenyl]-propionyl}-hydrazinecarboxylic acid tert-butyl ester
N-Butyl-3-[3-hydroxy-4-(1,1,4-trioxo-1,2,5-thiadiazolidin-2-yl)-phenyl]-propionamide
3-[3-Hydroxy-4-(1,1,4-trioxo-1,2,5-thiadiazolidin-2-yl)-phenyl]-N-pentylpropionamide
N-Hexyl-3-[3-hydroxy-4-(1,1,4-trioxo-1,2,5-thiadiazolidin-2-yl)-phenyl]-propionamide
3-[3-Hydroxy-4-(1,1,4-trioxo-1,2,5-thiadiazolidin-2-yl)-phenyl]-N-(4-phenylbutyl)-propionamide
3-[3-Hydroxy-4-(1,1,4-trioxo-1,2,5-thiadiazolidin-2-yl)-phenyl]-N-(5-phenylpentyl)-propionamide
N-(2-Hydroxyphenyl)-3-[3-hydroxy-4-(1,1,4-trioxo-1,2,5-thiadiazolidin-2-yl)-phenyl]-propionamide
3-[3-Hydroxy-4-(1,1,4-trioxo-1,2,5-thiadiazolidin-2-yl)-phenyl]-N-phenylpropionamide
3-[3-Hydroxy-4-(1,1,4-trioxo-1,2,5-thiadiazolidin-2-yl)-phenyl]-N-o-tolyl-propionamide
3-[3-Hydroxy-4-(1,1,4-trioxo-1,2,5-thiadiazolidin-2-yl)-phenyl]-N-isopropyl-propionamide
2-{3-[3-Hydroxy-4-(1,1,4-trioxo-1,2,5-thiadiazolidin-2-yl)-phenyl]-propionylamino}-2-methylpropionic acid
2-Hydroxy-6-(4-{3-[3-hydroxy-4-(1,1,4-trioxo-1,2,5-thiadiazolidin-2-yl)-phenyl]-propionylamino}-butoxy)-benzoic acid methyl ester
2-(4-{3-[3-Hydroxy-4-(1,1,4-trioxo-1,2,5-thiadiazolidin-2-yl)-phenyl]-propionylamino}-butoxy)-benzoic acid methyl ester
2-(4-{3-[3-Hydroxy-4-(1,1,4-trioxo-1,2,5-thiadiazolidin-2-yl)-phenyl]-propionylamino}-butoxy)-benzoic acid
3-[3-Hydroxy-4-(1,1,4-trioxo-1,2,5-thiadiazolidin-2-yl)-phenyl]-N-(4-phenoxybutyl)-propionamide
3-[3-Hydroxy-4-(1,1,4-trioxo-1,2,5-thiadiazolidin-2-yl)-phenyl]-N-[4-(2-trifluoromethylphenoxy)-butyl]-propionamide
3-[3-Hydroxy-4-(1,1,4-trioxo-1,2,5-thiadiazolidin-2-yl)-phenyl]-N-[4-(2-methanesulfonylphenoxy)-butyl]-propionamide
3-[3-Hydroxy-4-(1,1,4-trioxo-1,2,5-thiadiazolidin-2-yl)-phenyl]-N-[4-(3-methoxyphenoxy)-butyl]-propionamide
N-[4-(2,3-Dimethoxyphenoxy)-butyl]-3-[3-hydroxy-4-(1,1,4-trioxo-1,2,5-thiadiazolidin-2-yl)-phenyl]-propionamide
N-[4-(3-Hydroxyphenoxy)-butyl]-3-[3-hydroxy-4-(1,1,4-trioxo-1,2,5-thiadiazolidin-2-yl)-phenyl]-propionamide
N-[4-(2-Hydroxyphenoxy)-butyl]-3-[3-hydroxy-4-(1,1,4-trioxo-1,2,5-thiadiazolidin-2-yl)-phenyl]-propionamide
N-[4-(3-Hydroxy-2-methoxyphenoxy)-butyl]-3-[3-hydroxy-4-(1,1,4-trioxo-1,2,5-thiadiazolidin-2-yl)-phenyl]-propionamide
N-[4-(3-Hydroxy-2-methylphenoxy)-butyl]-3-[3-hydroxy-4-(1,1,4-trioxo-1,2,5-thiadiazolidin-2-yl)-phenyl]-propionamide
N-[4-(2-Acetyl-3-methoxyphenoxy)-butyl]-3-[3-hydroxy-4-(1,1,4-trioxo-1,2,5-thiadiazolidin-2-yl)-phenyl]-propionamide
2-Hydroxy-6-(4-{3-[3-hydroxy-4-(1,1,4-trioxo-1,2,5-thiadiazolidin-2-yl)-phenyl]-propionylamino}-butoxy)-N,N-dimethylbenzamide2-(4-{3-[3-Hydroxy-4-(1,1,4-trioxo-1,2,5-thiadiazolidin-2-yl)-phenyl]-propionylamino}-butoxy)-6,N,N-trimethyl benzamide
2-Fluoro-6-(4-{3-[3-hydroxy-4-(1,1,4-trioxo-1,2,5-thiadiazolidin-2-yl)-phenyl]-propionylamino}-butoxy)-N,N-dimethyl benzamide
2-Hydroxy-6-(4-{3-[3-hydroxy-4-(1,1,4-trioxo-1,2,5-thiadiazolidin-2-yl)-phenyl]-propionylamino}-butoxy)-benzoic acid
N-[4-(2-Acetyl-3-hydroxyphenoxy)-butyl]-3-[3-hydroxy-4-(1,1,4-trioxo-1,2,5-thiadiazolidin-2-yl)-phenyl]-propionamide
N-[4-(2-Cyano-3-hydroxyphenoxy)-butyl]-3-[3-hydroxy-4-(1,1,4-trioxo-1,2,5-thiadiazolidin-2-yl)-phenyl]-propionamide
N-[4-(3-Hydroxy-2-methanesulfinylphenoxy)-butyl]-3-[3-hydroxy-4-(1,1,4-trioxo-1,2,5-thiadiazolidin-2-yl)-phenyl]-propionamide
N-[4-(3-Hydroxy-2-methanesulfonylphenoxy)-butyl]-3-[3-hydroxy-4-(1,1,4-trioxo-1,2,5-thiadiazolidin-2-yl)-phenyl]-propionamide
2-(4-{2-Acetylamino-3-[3-hydroxy-4-(1,1,4-trioxo-1,2,5-thiadiazolidin-2-yl)-phenyl]-propionylamino}-butoxy)-6-hydroxybenzoic acid methyl ester
2-(4-{(S)-2-Acetylamino-3-[3-hydroxy-4-(1,1,4-trioxo-1,2,5-thiadiazolidin-2-yl)-phenyl]-propionylamino}-butoxy)-6-hydroxybenzoic acid methyl ester
3-[3-Hydroxy-4-(1,1,4-trioxo-1,2,5-thiadiazolidin-2-yl)-phenyl]-propionic acid methyl ester
3-[3-Hydroxy-4-(1,1,4-trioxo-1,2,5-thiadiazolidin-2-yl)-phenyl]-2-methylpropionic acid methyl ester
3-[3-Hydroxy-4-(1,1,4-trioxo-1,2,5-thiadiazolidin-2-yl)-phenyl]-2-methylpropionic acid tert-butyl ester
(1R*,2R*)-2-[3-Hydroxy-4-(1,1,4-trioxo-1,2,5-thiadiazolidin-2-yl)-phenyl]-cyclopropanecarboxylic acid ethyl ester
(1R*,2S*)-2-[3-Hydroxy-4-(1,1,4-trioxo-1,2,5-thiadiazolidin-2-yl)-phenyl]-cyclopropanecarboxylic acid ethyl ester
N-[3-Hydroxy-4-(1,1,4-trioxo-1,2,5-thiadiazolidin-2-yl)-benzyl]-N-methylbenzenesulfonamide
N-[3-Hydroxy-4-(1,1,4-trioxo-1,2,5-thiadiazolidin-2-yl)-benzyl]-N-methylmethanesulfonamide
C-Cyclohexyl-N-{2-[3-hydroxy-4-(1,1,4-trioxo-1,2,5-thiadiazolidin-2-yl)-phenyl]-ethyl}-methanesulfonamide
N-{2-[3-Hydroxy-4-(1,1,4-trioxo-1,2,5-thiadiazolidin-2-yl)-phenyl]-ethyl}-methansulfonamide
Ethanesulfonic acid {2-[3-hydroxy-4-(1,1,4-trioxo-1,2,5-thiadiazolidin-2-yl)-phenyl]-ethyl}-amide
Butane-1-sulfonic acid {2-[3-hydroxy-4-(1,1,4-trioxo-1,2,5-thiadiazolidin-2-yl)-phenyl]-ethyl}-amide
Propane-2-sulfonic acid {2-[3-hydroxy-4-(1,1,4-trioxo-1,2,5-thiadiazolidin-2-yl)-phenyl]-ethyl}-amide
Octane-1-sulfonic acid {2-[3-hydroxy-4-(1,1,4-trioxo-1,2,5-thiadiazolidin-2-yl)-phenyl]-ethyl}-amide
N-{2-[3-Hydroxy-4-(1,1,4-trioxo-1,2,5-thiadiazolidin-2-yl)-phenyl]-ethyl}-benzenesulfonamide
N-{2-[3-Hydroxy-4-(1,1,4-trioxo-1,2,5-thiadiazolidin-2-yl)-phenyl]-ethyl}-C-phenyl-methansulfonamide
4-Fluoro-N-{2-[3-hydroxy-4-(1,1,4-trioxo-1,2,5-thiadiazolidin-2-yl)-phenyl]-ethyl}-benzenesulfonamide
3,4-Dichloro-N-{2-[3-hydroxy-4-(1,1,4-trioxo-1,2,5-thiadiazolidin-2-yl)-phenyl]-ethyl}-benzenesulfonamide
3-(4-{2-[3-Hydroxy-4-(1,1,4-trioxo-1,2,5-thiadiazolidin-2-yl)-phenyl]-ethylsulfamoyl}-phenyl)-propionic acid
2-Hydroxy-5-{2-[3-hydroxy-4-(1,1,4-trioxo-1,2,5-thiadiazolidin-2-yl)-phenyl]-ethylsulfamoyl}-benzoic acid
Naphthalene-1-sulfonic acid {2-[3-hydroxy-4-(1,1,4-trioxo-1,2,5-thiadiazolidin-2-yl)-phenyl]-ethyl}-amide
2-Naphthalen-1-yl-ethanesulfonic acid {2-[3-hydroxy-4-(1,1,4-trioxo-1,2,5-thiadiazolidin-2-yl)-phenyl]-ethyl}-amide
N-{3-[3-Hydroxy-4-(1,1,4-trioxo-1,2,5-thiadiazolidin-2-yl)-phenyl]-propyl}-methansulfonamide
N-{3-[3-Hydroxy-4-(1,1,4-trioxo-1,2,5-thiadiazolidin-2-yl)-phenyl]-propyl}-benzenesulfonamide
N-{3-[3-Hydroxy-4-(1,1,4-trioxo-1,2,5-thiadiazolidin-2-yl)-phenyl]-propyl}-C-phenylmethanesulfonamide
C-(4-Fluorophenyl)-N-{3-[3-hydroxy-4-(1,1,4-trioxo-1,2,5-thiadiazolidin-2-yl)-phenyl]-propyl}-methanesulfonamide
N-{3-[3-Hydroxy-4-(1,1,4-trioxo-1,2,5-thiadiazolidin-2-yl)-phenyl]-propyl}-4-isopropylbenzenesulfonamide
N-{3-[3-Hydroxy-4-(1,1,4)-trioxo-1,2,5-thiadiazolidin-2-yl)-phenyl]-propyl}-4-trifluoromethylbenzenesulfonamide
N-{3-[3-Hydroxy-4-(1,1,4-trioxo-1,2,5-thiadiazolidin-2-yl)-phenyl]-propyl}-4-trifluoromethoxybenzenesulfonamide
C-(3-Aminophenyl)-N-{3-[3-hydroxy-4-(1,1,4-trioxo-1,2,5-thiadiazolidin-2-yl)-phenyl]-propyl}-methanesulfonamide
N-{3-[3-Hydroxy-4-(1,1,4-trioxo-1,2,5-thiadiazolidin-2-yl)-phenyl]-propyl}-2,4,6-triisopropylbenzenesulfonamide
2-Hydroxy-5-{3-[3-hydroxy-4-(1,1,4-trioxo-1,2,5-thiadiazolidin-2-yl)-phenyl]-propylsulfamoyl}-benzoic acid
3-Amino-N-{3-[3-hydroxy-4-(1,1,4-trioxo-1,2,5-thiadiazolidin-2-yl)-phenyl]-propyl}-benzenesulfonamide
4-Amino-N-{3-[3-hydroxy-4-(1,1,4-trioxo-1,2,5-thiadiazolidin-2-yl)-phenyl]-propyl}-benzenesulfonamide
N-{3-[3-Hydroxy-4-(1,1,4-trioxo-1,2,5-thiadiazolidin-2-yl)-phenyl]-propyl}-3,5-dimethylbenzenesulfonamide
N-{3-[3-Hydroxy-4-(1,1,4-trioxo-1,2,5-thiadiazolidin-2-yl)-phenyl]-propyl}-2,5-dimethylbenzenesulfonamide
N-{3-[3-Hydroxy-4-(1,1,4-trioxo-1,2,5-thiadiazolidin-2-yl)-phenyl]-propyl}-2,4,6-trimethylbenzenesulfonamide
4-tert-Butyl-N-{3-[3-hydroxy-4-(1,1,4-trioxo-1,2,5-thiadiazolidin-2-yl)-phenyl]-propyl}-benzenesulfonamide
4-(1,1-Dimethylpropyl)-N-{3-[3-hydroxy-4-(1,1,4-trioxo-1,2,5-thiadiazolidin-2-yl)-phenyl]-propyl}-benzenesulfonamide
N-{3-[3-Hydroxy-4-(1,1,4-trioxo-1,2,5-thiadiazolidin-2-yl)-phenyl]-propyl-}-3,4-dimethoxybenzenesulfonamide
N-{3-[3-Hydroxy-4-(1,1,4-trioxo-1,2,5-thiadiazolidin-2-yl)-phenyl]-propyl}-2,5-bis-(2,2,2-trifluoroethoxy)-benzenesulfonamide
Biphenyl-4-sulfonic acid {3-[3-hydroxy-4-(1,1,4-trioxo-1,2,5-thiadiazolidin-2-yl)-phenyl]-propyl}-amide
N-{3-[3-Hydroxy-4-(1,1,4-trioxo-1,2,5-thiadiazolidin-2-yl-phenyl]-propyl}-2-phenoxybenzenesulfonamide
N-{3-[3-Hydroxy-4-(1,1,4-trioxo-1,2,5-thiadiazolidin-2-yl)-phenyl]-propyl}-3-phenoxybenzenesulfonamide
N-{3-[3-Hydroxy-4-(1,1,4-trioxo-1,2,5-thiadiazolidin-2-yl)-phenyl]-propyl}-2,5-bis-(2,2,2-trifluoroethoxy)-benzenesulfonamide
2,2-Diphenylethanesulfonic acid {3-[3-hydroxy-4-(1,1,4-trioxo-1,2,5-thiadiazolidin-2-yl)-phenyl]-propyl}-amide
C-(2-Aminophenyl)-N{3-[3-hydroxy-4-(1,1,4-trioxo-1,2,5-thiadiazolidin-2-yl)-phenyl]-propyl}-methanesulfonamide
Naphthalene-1-sulfonic acid {3-[3-hydroxy-4-(1,1,4-trioxo-1,2,5-thiadiazolidin-2-yl)-phenyl]-propyl}-amide
C-Cyclohexyl-N-{3-[3-hydroxy-4-(1,1,4-trioxo-1,2,5-thiadiazolidin-2-yl)-phenyl]-propyl}-methanesulfonamide
2-Naphthalen-1-yl-ethanesulfonic acid {3-[3-hydroxy-4-(1,1,4-trioxo-1,2,5-thiadiazolidin-2-yl)-phenyl]-propyl}-amide
2-Phenyl-2-(2-trifluoromethylphenyl)-ethanesulfonic acid {3-[3-hydroxy-4-(1,1,4-trioxo-1,2,5-thiadiazolidin-2-yl)-phenyl]-propyl}-amide
2-Oxo-2H-chomene-6-sulfonic acid {3-[3-hydroxy-4-(1,1,4-trioxo-1,2,5-thiadiazolidin-2-yl)-phenyl]-propyl}-amide
N-{3-[3-Hydroxy-4-(1,1,4-trioxo-1,2,5-thiadiazolidin-2-yl)-phenylpropyl}-N-isopropylbenzenesulfonamide
N-(1-{2-[3-Hydroxy-4-(1,1,4-trioxo-1,2,5-thiadiazolidin-2-yl)-phenyl]-ethyl}-cyclopropyl)-benzenesulfonamide
N-{(S)-1-Benzyl-2-[3-hydroxy-4-(1,1,4-trioxo-1,2,5-thiadiazolidin-2-yl)-phenyl]-ethyl}-methanesulfonamide
Ethanesulfonic acid {(S)-1-benzyl-2-[3-hydroxy-4-(1,1,4-trioxo-1,2,5-thiadiazolidin-2-yl)-phenyl]-ethyl}-amide
N-{(S)-1-Benzyl-2-[3-hydroxy-4-(1,1,4-trioxo-1,2,5-thiadiazolidin-2-yl)-phenyl]-ethyl}-C-phenyl-methanesulfonamide
N-{(R)-1-Benzyl-2-[3-hydroxy-4-(1,1,4-trioxo-1,2,5-thiadiazolidin-2-yl)-phenyl]-ethyl}-C-phenyl-methanesulfonamide
N-{4-[3-Hydroxy-4-(1,1,4-trioxo-1,2,5-thiadiazolidin-2-yl)-phenyl]-butyl}-methanesulfonamide
N-{5-[3-Hydroxy-4-(1,1,4-trioxo-1,2,5-thiadiazolidin-2-yl)-phenyl]-pentyl}-methanesulfonamide
5-[2-Hydroxy-4-(1-methanesulfonylpiperidin-3-ylmethyl)-phenyl]-1,1-dioxo-1,2,5-thiadiazolidin-3-one
5-{2-Hydroxy-4-[2-(1-methanesulfonylpiperidin-2-yl)-ethyl]-phenyl}-1,1-dioxo-1,2,5-thiadiazolidin-3-one
5-{4-[2-(1-Benzenesulfonylpiperidin-2-yl)-ethyl]-2-hydroxyphenyl}-1,1-dioxo-1,2,5-thiadiazolidin-3-one
5-{4-[2-((S)-1-Benzenesulfonylpiperidin-2-yl)-ethyl]-2-hydroxyphenyl}-1,1-dioxo-1,2,5-thiadiazolidin-3-one
5-{4-[2-((R)-1-Benzenesulfonylpiperidin-2-yl)-ethyl]-2-hydroxyphenyl}-1,1-dioxo-1,2,5-thiadiazolidin-3-one
5-{4-[2-(1-Benzenesulfonylpyrrolidin-2-yl)-ethyl]-2-hydroxyphenyl}-1,1-dioxo-1,2,5-thiadiazolidin-3-one
5-{4-[2-(1-Benzenesulfonyl-1H-pyrrol-2-yl)-ethyl]-2-hydroxyphenyl}-1,1-dioxo-1,2,5-thiadiazolidin-3-one
5-{4-[2-(1-Benzenesulfonylpyrrolidin-3-yl)-ethyl]-2-hydroxyphenyl}-1,1-dioxo-1,2,5-thiadiazolidin-3-one
5-{4-[2-(1-Benzenesulfonylazepan-2-yl)-ethyl]-2-hydroxyphenyl}-1,1-dioxo-1,2,5-thiadiazolidin-3-one
5-{2-Hydroxy-4-[2-((R)-2-methanesulfonyl-1,2,3,4-tetrahydroisoquinolin-3-yl)-ethyl]-phenyl}-1,1-dioxo-1,2,5-thiadiazolidin-3-one
5-{4-[2-((R)-2-Benzenesulfonyl-1,2,3,4-tetrahydroisoquinolin-3-yl)-ethyl]-2-hydroxyphenyl}-1,1-dioxo-1,2,5-thiadiazolidin-3-one
5-(2-Hydroxy-4-{2-[2-(4-trifluoromethylbenzenesulfonyl)-1,2,3,4-tetrahydroisoquinolin-3-yl]-ethyl}-phenyl)-1,1-dioxo-1,2,5-thiadiazolidin-3-one
5-{2-Hydroxy-4-[2-(2-phenylmethanesulfonyl-1,2,3,4-tetrahydroisoquinolin-3-yl)-ethyl]-phenyl}-1,1-dioxo-1,2,5-thiadiazolidin-3-one
5-{4-[2-(1,1-Dioxo-1,2-thiazinan-3-yl)-ethyl]-2-hydroxyphenyl}-1,1-dioxo-1,2,5-thiadiazolidin-3-one
N-{(1R*,2S*)-2-[3-Hydroxy-4-(1,1,4-trioxo-1,2,5-thiadiazolidin-2-yl)-benzyl]-cyclohexyl}-methanesulfonamide
N-{(1R,2S)-2-[3-Hydroxy-4-(1,1,4-trioxo-1,2,5-thiadiazolidin-2-yl)-benzyl]-cyclohexyl}-methanesulfonamide
N-{(1S,2R)-2-[3-Hydroxy-4-(1,1,4-trioxo-1,2,5-thiadiazolidin-2-yl)-benzyl]-cyclohexyl}-methanesulfonamide
Ethanesulfonic acid {(1R*,2S*)-2-[3-hydroxy-4-(1,1,4-trioxo-1,2,5-thiadiazolidin-2-yl)-benzyl]-cyclohexyl}-amide N-{(1R*,2S*)-2-[3-Hydroxy-4-(1,1,4-trioxo-1,2,5-thiadiazolidin-2-yl)-benzyl]-cyclohexyl}-benzenesulfonamide
(S)-2-Benzenesulfonylamino-3-[3-hydroxy-4-(1,1,4-trioxo-1,2,5-thiadiazolidin-2-yl)-phenyl]-N-pentylpropionamide
(S)-2-Benzenesulfonylamino-3-[3-hydroxy-4-(1,1,4-trioxo-1,2,5-thiadiazolidin-2-yl)-phenyl]-N-(4-phenylbutyl)-propionamide
N-{(S)-1-(1H-Benzoimidazol-2-yl)-2-[3-hydroxy-4-(1,1,4-trioxo-1,2,5-thiadiazolidin-2-yl)-phenyl]-ethyl}-benzenesulfonamide
tert-Butyl [({2-[4-(1,1-dioxido-4-oxo-1,2,5-thiadiazolidin-2-yl)-3-hydroxyphenyl]ethyl}amino)sulfonyl]carbamate
1-Cyclohexyl-3-{2-[3-hydroxy-4-(1,1,4-trioxo-1,2,5-thiadiazolidin-2-yl)-phenyl]-ethyl}-urea
1-{2-[3-Hydroxy-4-(1,1,4-trioxo-1,2,5-thiadiazolidin-2-yl)-phenyl]-ethyl}-3-phenyl-urea
1-Ethyl-3-{2-[3-hydroxy-4-(1,1,4-trioxo-1,2,5-thiadiazolidin-2-yl)-phenyl]-ethyl}-urea
1-Adamantan-1-yl-3-{2-[3-hydroxy-4-(1,1,4-trioxo-1,2,5-thiadiazolidin-2-yl)-phenyl]-ethyl}-urea
Benzenesulfonyl-N-{2-[3-hydroxy-4-(1,1,4-trioxo-1,2,5-thiadiazolidin-2-yl)-phenyl]-ethyl}-urea
1-(2,4-Dimethoxybenzyl)-3-{2-[3-hydroxy-4-(1,1,4-trioxo-1,2,5-thiadiazolidin-2-yl)-phenyl]-ethyl}-urea
1-(2-Hydroxyethyl)-3-{2-[3-hydroxy-4-(1,1,4-trioxo-1,2,5-thiadiazolidin-2-yl)-phenyl]-ethyl}-urea
3-{2-[3-Hydroxy-4-(1,1,4-trioxo-1,2,5-thiadiazolidin-2-yl)-phenyl]-ethyl}-1,1-bis-(2-methoxyethyl)-urea
Morpholine-4-carboxylic acid {2-[3-hydroxy-4-(1,1,4-trioxo-1,2,5-thiadiazolidin-2-yl)-phenyl]-ethyl}-amide
4-(3-{2-[3-Hydroxy-4-(1,1,4-trioxo-1,2,5-thiadiazolidin-2-yl)-phenyl]-ethyl}-ureido)-piperidine-1-carboxylic acid tert-butyl ester
1-{2-[3-Hydroxy-4-(1,1,4-trioxo-1,2,5-thiadiazolidin-2-yl)-phenyl]-ethyl}-3-piperidin-4-yl-urea
1-{3-[3-Hydroxy-4-(1,1,4-trioxo-1,2,5-thiadiazolidin-2-yl)-phenyl]-propyl}-3-phenyl-urea
1-Cyclohexyl-3-{3-[3-hydroxy-4-(1,1,4-trioxo-1,2,5-thiadiazolidin-2-yl)-phenyl]-propyl}-urea
1-Adamantan-1-yl-3-{3-[3-hydroxy-4-(1,1,4-trioxo-1,2,5-thiaidiazolidin-2-yl)-phenyl]-propyl}-urea
3-{3-[3-Hydroxy-4-(1,1,4-trioxo-1,2,5-thiadiazolidin-2-yl)-phenyl]-propyl}-1H-quinazoline-2,4-dione
3-[3-Hydroxy-4-(1,1,4-trioxo-1,2,5-thiadiazolidin-2-yl)-benzyl]-piperidine-1-carboxylic acid ethylamide
5-(2-Hydroxy-4-methanesulfonylmethylphenyl)-1,1-dioxo-1,2,5-thiadiazolidin-3-one
5-(4-Ethanesulfonylmethyl-2-hydroxy-phenyl)-1,1-dioxo-1,2,5-thiadiazolidin-3-one
5-[2-Hydroxy-4-(propane-2-sulfonylmethyl)-phenyl]-1,1-dioxo-1,2,5-thiadiazolidin-3-one
5-(4-Benzenesulfonylmethyl-2-hydroxyphenyl)-1,1-dioxo-1,2,5-thiadiazolidin-3-one
5-(2-Hydroxy-4-methanesulfinylmethylphenyl)-1,1-dioxo-1,2,5-thiadiazolidin-3-one
5-(4-Ethanesulfinylmethyl-2-hydroxyphenyl)-1,1-dioxo-1,2,5-thiadiazolidin-3-one
5-[2-Hydroxy-4-(propane-2-sulfinylmethyl)-phenyl]-1,1-dioxo-1,2,5-thiadiazolidin-3-one
5-(2-Hydroxy-4-methylsulfanylmethylphenyl)-1,1-dioxo-1,2,5-thiadiazolidin-3-one
5-(4-Ethylsulfanylmethyl-2-hydroxyphenyl)-1,1-dioxo-1,2,5-thiadiazolidin-3-one
5-(2-Hydroxy-4-isopropylsulfanylmethylphenyl)-1,1-dioxo-1,2,5-thiadiazolidin-3-one
5-[4-(2-Benzenesulfonylethyl)-2-hydroxyphenyl]-1,1-dioxo-1,2,5-thiadiazolidin-3-one
5-[4-(4-Benzenesulfonylbutyl)-2-hydroxyphenyl]-1,1-dioxo-1,2,5-thiadiazolidin-3-one
5-{4-[3-(1,1-Dioxotetrahydrothiophen-2-yl)-prop-1-ynyl]-2-hydroxyphenyl}-1,1-dioxo-1,2,5-thiadiazolidin-3-one
5-{4-[3-(1,1-Dioxotetrahydrothiophen-2-yl)-propyl]-2-hydroxyphenyl}-1,1-dioxo-1,2,5-thiadiazolidin-3-one
5-[2-Hydroxy-4-(3-oxopentyl)-phenyl]-1,1-dioxo-1,2,5-thiadiazolidin-3-one
5-[2-Hydroxy-4-(2-methyl-3-oxopentyl)-phenyl]-1,1-dioxo-1,2,5-thiadiazolidin-3-one
5-[2-Hydroxy-4-(2-methyl-3-oxo-3-phenylpropyl)-phenyl]-1,1-dioxo-1,2,5-thiadiazolidin-3-one
5-[4-(2-Benzoylbutyl)-2-hydroxyphenyl]-1,1-dioxo-1,2,5-thiadiazolidin-3-one
5-[4-(2-Benzoylpentyl)-2-hydroxyphenyl]-1,1-dioxo-1,2,5-thiadiazolidin-3-one
5-[2-Hydroxy-4-(3-oxo-2,3-diphenylpropyl)-phenyl]-1,1-dioxo-1,2,5-thiadiazolidin-3-one
5-[4-(2-Benzyl-3-oxo-3-phenylpropyl)-2-hydroxyphenyl]-1,1-dioxo-1,2,5-thiadiazolidin-3-one
5-[4-(2,2-Dimethyl-3-oxo-3-phenylpropyl)-2-hydroxyphenyl]-1,1-dioxo-1,2,5-thiadiazolidin-3-one
5-[2-Hydroxy-4-(1-oxo-indan-2-ylmethyl)-phenyl]-1,1-dioxo-1,2,5-thiadiazolidin-3-one
5-[2-Hydroxy-4-(6-oxo-6,7,8,9-tetrahydro-5H-benzocyclohepten-5-ylmethyl)-phenyl]-1,1-dioxo-1,2,5-thiadiazolidin-3-one
5-[2-Hydroxy-4-(2-methoxy-3-oxo-3-phenylpropyl)-phenyl]-1,1-dioxo-1,2,5-thiadiazolidin-3-one
5-[2-Hydroxy-4-(3-hydroxy-2-methyl-3-phenylpropyl)-phenyl]-1,1-dioxo-1,2,5-thiadiazolidin-3-one
5-{2-Hydroxy-4-[2-(hydroxyphenylmethyl)-butyl]-phenyl}-1,1-dioxo-1,2,5-thiadiazolidin-3-one
5-{2-Hydroxy-4-[2-(hydroxyphenylmethyl)-pentyl]-phenyl}-1,1-dioxo-1,2,5-thiadiazolidin-3-one
5-[4-(2-Benzyl-3-hydroxy-3-phenylpropyl)-2-hydroxyphenyl]-1,1-dioxo-1,2,5-thiadiazolidin-3-one
5-[2-Hydroxy-4-(3-hydroxy-2,2-dimethyl-3-phenylpropyl)-phenyl]-1,1-dioxo-1,2,5-thiadiazolidin-3-one
5-[2-Hydroxy-4-(1-hydroxyindan-2-ylmethyl)-phenyl]-1,1-dioxo-1,2,5-thiadiazolidin-3-one
5-[2-Hydroxy-4-(3-hydroxy-2-methoxy-3-phenyl-propyl)-phenyl]-1,1-dioxo-1,2,5-thiadiazolidin-3-one
5-(2-Hydroxy-4-vinylphenyl)-1,1-dioxo-1,2,5-thiadiazolidin-3-one
5-[2-Hydroxy-4-(1-hydroxyethyl)-phenyl]-1,1-dioxo-1,2,5-thiadiazolidin-3-one
5-[2-Hydroxy-4-(2-hydroxyhexyl)-phenyl]-1,1-dioxo-1,2,5-thiadiazolidin-3-one
5-[2-Hydroxy-4-(3-hydroxybutyl)-phenyl]-1,1-dioxo-1,2,5-thiadiazolidin-3-one
5-{2-Hydroxy-4-[2-(1-hydroxycyclohexyl)-ethyl]-phenyl}-1,1-dioxo-1,2,5-thiadiazolidin-3-one
5-[2-Hydroxy-4-(4,4,4-trifluoro-3-hydroxy-3-phenylbutyl)-phenyl]-1,1-dioxo-1,2,5-thiadiazolidin-3-one
5-(3-Hydroxybiphenyl-4-yl)-1,1-dioxo-1,2,5-thiadiazolidin-3-one
5-(3,3′-Dihydroxybiphenyl-4-yl)-1,1-dioxo-1,2,5-thiadiazolidin-3-one
[3′-Hydroxy-4′-(1,1,4-trioxo-1,2,5-thiadiazolidin-2-yl)-biphenyl-4-yl]-acetic acid
5,5′-(3,3′-Dihydroxybiphenyl-4-yl)-1,1,1′,1′-tetraoxo-1,1′,2,2′,5,5′-dithiadiazolidin-3,3′-one
5-(4-Furan-3-yl-2-hydroxyphenyl)-1,1-dioxo-1,2,5-thiadiazolidin-3-one
5-(2-Hydroxy-4-thiophen-3-yl-phenyl)-1,1-dioxo-1,2,5-thiadiazolidin-3-one
5-(4-Benzofuran-3-yl-2-hydroxyphenyl)-1,1-dioxo-1,2,5-thiadiazolidin-3-one
5-[2-Hydroxy-4-(6-methoxybenzofuran-3-yl)-phenyl]-1,1-dioxo-1,2,5-thiadiazolidin-3-one
5-(2-Hydroxy-4-thiazol-5-yl-phenyl)-1,1-dioxo-1,2,5-thiadiazolidin-3-one
5-(2-Hydroxy-4-thiazol-2-yl-phenyl)-1,1-dioxo-1,2,5-thiadiazolidin-3-one
5-[2-Hydroxy-4-(1H-pyrrol-3-yl)-phenyl]-1,1-dioxo-1,2,5-thiadiazolidin-3-one
5-[2-Hydroxy-4-(1H-pyrazol-3-yl)-phenyl]-1,1-dioxo-1,2,5-thiadiazolidin-3-one
5-[2-Hydroxy-4-(1H-pyrazol-4-yl)-phenyl]-1,1-dioxo-1,2,5-thiadiazolidin-3-one
5-[2-Hydroxy-4-(1-propyl-1H-pyrazol-4-yl)-phenyl]-1,1-dioxo-1,2,5-thiadiazolidin-3-one
5-[2-Hydroxy-4-(1-isobutyl-1H-pyrazol-4-yl)-phenyl]-1,1-dioxo-1,2,5-thiadiazolidin-3-one
5-{2-Hydroxy-4-[1-(3-methylbutyl)-1H-pyrazol-4-yl]-phenyl}-1,1-dioxo-1,2,5-thiadiazolidin-3-one
5-[2-Hydroxy-4-(tetrahydrofuran-3-yl)-phenyl]-1,1-dioxo-1,2,5-thiadiazolidin-3-one
5-[4-(2,3-Dihydrobenzofuran-3-yl)-2-hydroxyphenyl]-1,1-dioxo-1,2,5-thiadiazolidin-3-one
5-(2-Hydroxy-4-thiazol-2-ylmethylphenyl)-1,1-dioxo-1,2,5-thiadiazolidin-3-one
5-[2-Hydroxy-4-(2H-pyrazol-3-ylmethyl)-phenyl]-1,1-dioxo-1,2,5-thiadiazolidin-3-one
5-(2-Hydroxy-4-pyrazol-1-ylmethyl-phenyl)-1,1-dioxo-1,2,5-thiadiazolidin-3-one
5-[2-Hydroxy-4-(3-trifluoromethylpyrazole-1-ylmethyl)-phenyl]-1,1-dioxo-1,2,5-thiadiazolidin-3-one
5-[3-Hydroxy-4-(1,1,4-trioxo-1,2,5-thiadiazolidin-2-yl)-phenyl]-pentanoic acid
4-[3-Hydroxy-4-(1,1,4-trioxo-1,2,5-thiadiazolidin-2-yl)-phenyl]-butane-1-sulfinic acid
4-[3-Hydroxy-4-(1,1,4-trioxo-1,2,5-thiadiazolidin-2-yl)-phenyl]-butyronitrile
4-[3-Hydroxy-4-(1,1,4-trioxo-1,2,5-thiadiazolidin-2-yl)-phenyl]-2-methyl-butyronitrile
4-[3-Hydroxy-4-(1,1,4-trioxo-1,2,5-thiadiazolidin-2-yl)-phenyl]-3,3-dimethylbutyronitrile
[3-Hydroxy-4-(1,1,4-trioxo-1,2,5-thiadiazolidin-2-yl)-phenoxy]-acetic acid 2-trimethylsilanylethyl ester
[3-Hydroxy-4-(1,1,4-trioxo-1,2,5-thiadiazolidin-2-yl)-phenoxy]-acetic acid
3-[3-Hydroxy-4-(1,1,4-trioxo-1,2,5-thiadiazolidin-2-yl)-phenoxy]-1,3,4,5-tetrahydro-benzo[b]azepin-2-one
5-(4-Ethyl-2-hydroxyphenyl)-1,1-dioxo-1,2,5-thiadiazolidin-3-one
5-(4-Hexyl-2-hydroxyphenyl)-1,1-dioxo-1,2,5-thiadiazolidin-3-one
5-(2-Hydroxy-4-isobutylphenyl)-1,1-dioxo-1,2,5-thiadiazolidin-3-one
5-[4-(3,3-Dimethylbutyl)-2-hydroxyphenyl]-1,1-dioxo-1,2,5-thiadiazolidin-3-one
5-[2-Hydroxy-4-(3,3,3-trifluoropropyl)-phenyl]-1,1-dioxo-1,2,5-thiadiazolidin-3-one
5-(4-Cyclopentylmethyl-2-hydroxyphenyl)-1,1-dioxo-1,2,5-thiadiazolidin-3-one
5-(4-Cyclohexylmethyl-2-hydroxyphenyl)-1,1-dioxo-1,2,5-thiadiazolidin-3-one
5-{2-Hydroxy-4-[1-(2,4,6-trimethylphenyl)-ethyl]-phenyl}-1,1-dioxo-1,2,5-thiadiazolidin-3-one
5-[4-(2-Aminobenzyl)-2-hydroxyphenyl]-1,1-dioxo-1,2,5-thiadiazolidin-3-one
5-[2-Hydroxy-4-(2-hydroxybenzyl)-phenyl]-1,1-dioxo-1,2,5-thiadiazolidin-3-one
5-[2-Hydroxy-4-(2-hydroxy-5-methylbenzyl)-phenyl]-1,1-dioxo-1,2,5-thiadiazolidin-3-one
5-[4-(2-Aminomethylbenzyl)-2-hydroxyphenyl]-1,1-dioxo-1,2,5-thiadiazolidin-3-one
5-[2-Hydroxy-4-(2-methoxymethylbenzyl)-phenyl]-1,1-dioxo-1,2,5-thiadiazolidin-3-one
{2-[3-Hydroxy-4-(1,1,4-trioxo-1,2,5-thiadiazolidin-2-yl)-benzyl]-phenyl}-acetonitrile
{2-[3-Hydroxy-4-(1,1,4-trioxo-1,2,5-thiadiazolidin-2-yl)-benzyl]-phenyl}-acetic acid methyl ester
{2-[3-Hydroxy-4-(1,1,4-trioxo-1,2,5-thiadiazolidin-2-yl)-benzyl]-phenyl}-acetic acid
N-Ethyl-2-{2-[3-hydroxy-4-(1,1,4-trioxo-1,2,5-thiadiazolidin-2-yl)-benzyl]-phenyl}-acetamide
5-(2-Hydroxy-4-{2-[2-(4-methylpiperidin-1-yl)-2-oxo-ethyl]-benzyl}-phenyl)-1,1-dioxo-1,2,5-thiadiazolidin-3-one
5-{2-Hydroxy-4-[2-(2-hydroxyethyl)-benzyl]-phenyl}-1,1-dioxo-1,2,5-thiadiazolidin-3-one
5-[2-Hydroxy-4-(pyridine-2-carbonyl)-phenyl]-1,1-dioxo-1,2,5-thiadiazolidin-3-one
5-(4-Benzenesulfonyl-2-hydroxyphenyl)-1,1-dioxo-1,2,5-thiadiazolidin-3-one
5-(2-Hydroxy-4-trifluoromethylphenyl)-1,1-dioxo-1,2,5-thiadiazolidin-3-one
5-(2-Hydroxy-4-methoxyphenyl)-1,1-dioxo-1,2,5-thiadiazolidin-3-one
3-Hydroxy-4-(1,1,4-trioxo-1,2,5-thiadiazolidin-2-yl)-benzonitrile, and
5-(4-Chloro-2-hydroxyphenyl)-1,1-dioxo-1,2,5-thiadiazolidin-3-one
   or a pharmaceutically acceptable salt thereof.

1. A method of promoting physiological cardiac hypertrophy in an individual, the method comprising
identifying an individual having or suspected of having pathological cardiac hypertrophy; and
contacting the cardiomyocytes of the individual with a therapeutically effective amount of a PTP inhibitor sufficient to promote physiological hypertrophy.

2. (canceled)

3. The method according to claim 1, wherein the individual is diagnosed with heart failure.

4. The method according to claim 1, wherein the individual is diagnosed with diastolic dysfunction.

5. The method according to claim 1, wherein the individual is diagnosed with myocardial infarction.

6. The method according to claim 1, wherein the individual is diagnosed with dilated, familial, or ischemic cardiomyopathy.

7. The method of claim 1, wherein the contacting is accomplished by systemic administration of the inhibitor to the individual.

8. The method of claim 7, wherein the systemic administration comprises oral administration.

9. The method or use of claim 7, wherein the systemic administration comprises intravenous administration.

10. The method of claim 1, wherein the contacting is accomplished by administration directly to the heart

11. The method of claim 10, wherein the administration is accomplished by direct injection to the heart muscle.

12. The method of claim 10, wherein the administration is accomplished by use of a catheter in a coronary artery that supplies the heart muscle.

13. The method according to claim 1, wherein the PTP inhibitor is a compound of the formula 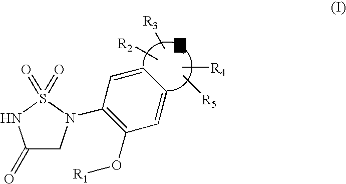
wherein
Q combined together with the carbon atoms to which it is attached form an aromatic, or a partially or fully saturated nonaromatic 5- to 8-membered carbocyclic or heterocyclic ring;
R₁ is hydrogen, —C(O)R₆, —C(O)NR₇R₈ or —C(O)OR₉ in which
R₆ and R₇ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₈ and R₉ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocycyl;
R₂, R₃, R₄ and R₅ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 3- to 7-membered fused ring; or
R₂ and R₃ combined are alkylene which together with the carbon atom to which they are attached form a 3- to 7-membered spirocyclic ring;
or a pharmaceutically acceptable salt thereof.

14. The method according to claim 1, wherein the PTP inhibitor is a compound of the formula 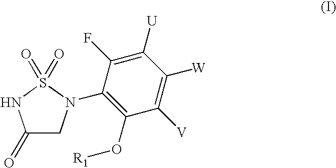
wherein
R₁ is hydrogen, —C(O)R₂, —C(O)NR₃R₄ or —C(O)OR₅ in which
R₂ and R₃ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₄ and R₅ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocycyl;
U, W and V are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, aryloxy, arylthio, heterocycyl, heterocyclyloxy, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
U and W combined together with the carbon atoms to which they are attached form an optionally substituted aromatic, or a partially or fully saturated nonaromatic 5- to 8-membered carbocyclic or heterocyclic ring; or
W and V combined together with the carbon atoms to which they are attached form an optionally substituted aromatic, or partially or fully saturated nonaromatic 5- to 8-membered carbocyclic or heterocyclic ring;
or a pharmaceutically acceptable salt thereof.

15. The method according to claim 1, wherein the PTP inhibitor is a compound of the formula 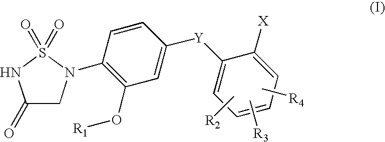
wherein
R₁ is hydrogen, —C(O)R₅, —C(O)NR₆R₇ or —C(O)OR₈ in which
R₅ and R₆ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₇ and R₈ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocycyl;
R₂, R₃ and R₄ are, independently from each other, hydrogen, hydroxy, halogen, cyano, nitro, alkoxy, alkylthio, alkylthiono, sulfonyl, free or esterified carboxy, carbamoyl, sulfamoyl, optionally substituted amino, cycloalkyl, aryl, heterocyclyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₂ and R₃ combined are alkylene which together with the ring atoms to which they are attached form a 5- to 7-membered fused ring provided R₂ and R₃ are attached to carbon atoms adjacent to each other; or
R₂ and R₃ combined together with the carbon atom to which they are attached form a fused 5- to 6-membered aromatic or heteroaromatic ring provided R₂ and R₃ are attached to carbon atoms adjacent to each other;
X is hydrogen, fluoro, cyano, or free or esterified carboxy; or
X is —NR₉C(O)R₁₀, —NR₉C(O)OR₁₁, —NR₉S(O)₂R₁₂, —(CH₂)ₘS(O)₂R₁₃, —OS(O)₂R₁₄ or —OₙC(O)NR₁₅R₁₆ in which
R₉ is hydrogen, lower alkyl, acyl, alkoxycarbonyl or sulfonyl;
R₁₀, R₁₁, R₁₂, R₁₃ and R₁₄ are, independently from each other, cycloalkyl, aryl, heterocyclyl, aralkyl, heteroaralkyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, amino, alkylamino, dialkylamino, acylamino, carbamoyl, thiol, alkylthio, alkylthiono, sulfonyl, sulfonamido, sulfamoyl, nitro, cyano, free or esterified carboxy, aryl, aryloxy, arylthio, alkenyl, alkynyl, aralkoxy, heteroaralkoxy, heterocyclyl and heterocyclyloxy; or
R₁₀, R₁₂ and R₁₃ are, independently from each other, —NR₁₇R₁₈ in which
R₁₇ and R₁₈ are, independently from each other, hydrogen, alkyl, cycloalkyl, aralkyl, aryl or heterocyclyl; or
R₁₇ and R₁₈ combined are alkylene which together with the nitrogen atom to which they are attached form a 4- to 7-membered ring;
R₁₅ and R₁₆ are, independently from each other, hydrogen, alkyl, cycloalkyl, aralkyl, aryl or heterocyclyl; or
R₁₅ and R₁₆ combined are alkylene which together with the nitrogen atom to which they are attached form a 4- to 7-membered ring;
m and n are, independently from each other, zero or an integer of 1; or
C—X is replaced by nitrogen;
Y is CH₂, O or S;
or a pharmaceutically acceptable salt thereof.

16. The method according to claim 1, wherein the PTP inhibitor is a compound of the formula 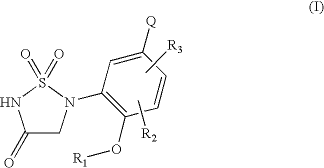
wherein
Q is alkoxy, alkylthio, alkylthiono, sulfonyl, cycloalkyl, aryl, aryloxy, heterocycyl, alkenyl, alkynyl or (C₁₋₈)alkyl optionally substituted with one to four substituents selected from the group consisting of halogen, hydroxy, cycloalkyl, cycloalkoxy, acyl, acyloxy, alkoxy, alkyloxyalkoxy, optionally substituted amino, carbamoyl, thiol, alkylthio, alkylthiono, sulfonyl, sulfamoyl, nitro, cyano, free or esterified carboxy, aryl, aryloxy, arylthio, alkenyl, alkynyl, aralkoxy, heteroaralkoxy, heterocyclyl and heterocylyloxy;
R₁ is hydrogen, —C(O)R₄, —C(O)NR₅R₇ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocycyl;
R₂ and R₃ are, independently from each other, hydrogen, halogen, (C₁₋₃)alkyl or (C₁₋₃)alkoxy;
or a pharmaceutically acceptable salt thereof.

17. The method according to claim 1, wherein the PTP inhibitor is a compound of the formula 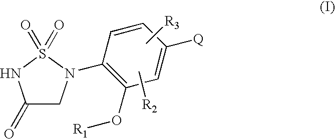
wherein
Q is:
i) —X, or
ii)-Y—(CH₂)ₙ—(CR₈R₉)ₚ—(CH₂)ₘ-Z-X in which;
Y is oxygen or S(O)_{q} in which q is zero or an integer of 1 or 2; or
Y is —C≡C— or —C═C—; or
Y is cyclopropyl or
Y is absent;
n and m are, independently from each other, zero or an integer from 1 to 8;
R₈ and R₉ are, independently from each other, hydrogen, hydroxyl, alkoxy, alkanoyl, alkanoylamino, alkoxycarbonyl, aralkyl, heteroaryl, carbamoyl, aryl, or alkyl; or
R₈ and R₉ combined are alkylene which together with the carbon atom to which they are attached form a 3- to 7-membered ring;
p is zero or an integer selected from 1 or 2
Z is absent;
Z is —C(O)—O—; or
Z is —C(O)—; or
Z is —C(O)—NRα-alkylene- or —C(O)—NRα-alkylene-O—, wherein R [symbol] is H or lower alkyl; or
Z is —CO—NRα-(CH₂)_{n′}—(CR_{8′}R_{9′})_{p′}—(CH₂)_{m′}, or —C(O)—NRα-(CH₂)_{n′}—(CR_{8′}R_{9′})_{p′}-(CH₂)_{m′}—O—, wherein p′ is zero or an integer of 1, n′ and m′ are, independently from each other, zero or an integer from 1 to 8, R_{8′} and R_{9′} are, independently from each other, hydrogen or lower alkyl, R□ is H or lower alkyl; or
Z is —NRα′-C(O)—, or —NRα′-C(O)—O—, wherein Rα′ is H or lower alkyl, or Rα′ and R₉ combined are alkylene which together with the carbon atom to which they are attached form a 3- to 7-membered ring; or
Z is —C(O)—NH—NH—C(O)—O—; or
Z is —S(O)₂—, or —S(O)—; or
Z is —NRβS(O)₂—, wherein Rβ is H, lower alkyl, or Rβ and R₉ combined are alkylene which together with the carbon atom to which they are attached form a 3- to 7-membered ring; or
Z is —NH—S(O)₂—NH—C(O)—O—; or
Z is —NRγ-C(O)—NRγ′-; wherein Rγ′ is H, alkyl, aryl, heterocyclyl, or lower alkoxy and Rγ is H, lower alkyl, or Rγ and R₉ combined are alkylene which together with the carbon atom to which they are attached form a 3- to 7-membered ring; or Rγ′ and X combined are alkylene which together with the carbon atom to which they are attached form a 3- to 7-membered ring or
Z is —NRτ-C(O)—NH—S(O)_{z}—, wherein Rτ is H or lower alkyl,
X is hydrogen, hydroxy, NH₂, halogen, alkoxy, alkylthio, alkyl, —S(O)—OH, alkyl, cycloalkyl, cycloalkoxy, acyl, acyloxy, carbamoyl, optionally substituted amino, cyano, trifluoromethyl, free or esterified carboxy, heterocyclyl, heterocyclooxy, heteroaryl, heteroaralkyl, aryl, aralkyl, aralkoxy, aryloxy, aralkylthio, arylthio;
R₁ is hydrogen, —C(O)R₄, —C(O)NR₅R₆ or —C(O)OR₇ in which
R₄ and R₅ are, independently from each other, hydrogen, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocyclyl;
R₆ and R₇ are, independently from each other, cycloalkyl, aryl, heterocyclyl, aralkyl, heteroaralkyl or alkyl optionally substituted with one to four substituents selected from the group consisting of halogen, cycloalkyl, cycloalkoxy, alkoxy, alkyloxyalkoxy, amino, alkylamino, dialkylamino, aryl, aryloxy and heterocycyl;
R₂ and R₃ are, independents from each other, hydrogen, halogen, (C₁₋₃)alkyl or (C₁₋₃)alkoxy;
or a pharmaceutically acceptable salt thereof,
and wherein n+m+p is >1 or is 0, when X is aryl, and Y and Z are absent,
n+m+p is not 0 when X is —O-aryl, and Y and Z are absent, or
n+m+p is not 0 when X is —S-aryl, and Y and Z are absent, or
n+m+p is not 0 when X is —CH₂-aryl, and Y and Z are absent, or
n+m+p is not 0 when X is aryl, Z is absent and Y is —O— or Y is —S—, or
wherein Q cannot be —CH₂-aryl, —S-aryl or —O-aryl.

18. The method according to claim 1, further comprising administering an IGF1 molecule to the individual.

19. (canceled)
